(19) United States
(12) Patent Application Publication
(10) Pub. No.: US 2016/0138049 A1
(43) Pub. Date: May 19, 2016

(54) OXYGEN-TOLERANT CoA-ACETYLATING ALDEHYDE DEHYDROGENASE CONTAINING PATHWAY FOR BIOFUEL PRODUCTION

(71) Applicant: The Regents of the University of California, Oakland, CA (US)

(72) Inventors: James C. LIAO, Los Angeles, CA (US); Ethan I. LAN, Los Angeles, CA (US)

(21) Appl. No.: 14/896,246

(22) PCT Filed: Jun. 10, 2014
(86) PCT No.: PCT/US2014/041699

§ 371 Date: Dec. 4, 2015

Related U.S. Application Data
(60) Provisional application No. 61/833,325, filed on Jun. 10, 2013.

(51) Int. Cl. C12P 7/16; C12N 9/02; C12P 7/06; C12N 15/52; C12N 9/04; C10L 1/02; C12N 9/00

(52) U.S. Cl. CPC C12P 7/16; C10L 1/02; C12N 9/0008; C12N 9/93; C12N 15/52; C12N 9/0006; C12P 7/065; C10L 2200/0469; C12Y 602/01016; C12Y 102/0101; C12Y 604/01002; C12Y 101/01001

(57) ABSTRACT

Provided herein are metabolically-modified microorganisms useful for producing biofuels. More specifically, provided herein are methods of producing high alcohols including isobutanol, 1-butanol, 1-propanol, 2-methyl-1-butanol, 3-methyl-1-butanol and 2-phenylethanol from a suitable substrate.

### CROSS REFERENCE TO RELATED APPLICATIONS

This application claims priority to U.S. Provisional Application No. 61/833,325, filed Jun. 10, 2013, the disclosure of which is incorporated herein by reference in its entirety.

### BACKGROUND

According to the US Energy Information Administration (EIA, 2007), world energy-related CO₂ emissions in 2004 were 26,922 million metric tons and increased 26.7% from 1990. As a result, atmospheric levels of CO₂ have increased by about 25% over the past 150 years. Thus, it has become increasingly important to develop new technologies to reduce CO₂ emissions.

The world is also facing costly gas and oil and limited reserves of these precious resources. Biofuels have been recognized as an alternative energy source. While efforts have been made to improve various productions, further developments are needed.

### SUMMARY

Converting CO₂ or other carbon sources into 1-butanol, an important chemical feedstock and potential fuel, is an attractive strategy for tackling energy and environmental problems. The Coenzyme A (CoA) dependent pathway for the production of 1-butanol is the most energy efficient. The first step of the CoA pathway, condensation of two acetyl-CoA, is strongly thermodynamically unfavorable. Contrary to the conventional wisdom that energy efficiency is crucial to microbial production; the disclosure demonstrates that ATP consumption is beneficial for the direct photosynthetic production of 1-butanol from S. elongatus PCC 7942. Energy from ATP hydrolysis was incorporated into the CoA pathway to overcome the high thermodynamic barrier for biosynthesis of acetoacetyl-CoA, the first pathway intermediate. ATP activation of acetyl-CoA into malonyl-CoA and the subsequent decarboxylative carbon chain elongation mechanism found in fatty acid and polyketide synthesis was used to irreversibly drive the synthesis of acetoacetyl-CoA. By designing a novel malonyl-CoA dependent 1-butanol production pathway, direct photosynthetic production of 1-butanol from CO₂ was obtained. In addition, the disclosure demonstrates the substitution of bifunctional aldehyde/alcohol dehydrogenase (AdhE2) with separate oxygen-tolerant CoA-acylating aldehyde dehydrognease (PduP) and alcohol dehydrogenase (YqhD) increases the 1-butanol production.

The disclosure provides a novel malonyl-CoA dependent 1-butanol pathway and demonstrates the direct photosynthetic production of 1-butanol from S. elongatus PCC 7942 under oxygenic condition. Contrary to the notion that energy efficiency is important for microbial production, the consumption of ATP is beneficial for cyanobacteria to produce 1-butanol. ATP hydrolysis was used to drive the formation of acetoacetyl-CoA. The release of free energy from ATP hydrolysis is used to overcome the thermodynamically unfavorable condensation of two acetyl-CoA. To incorporate energy of ATP hydrolysis into the CoA 1-butanol pathway, malonyl-CoA biosynthesis was used in combination with the decarboxylative carbon chain elongation using malonyl-CoA found in fatty acid and polyketide synthesis to irreversibly trap carbon flux into the formation of acetoacetyl-CoA. Despite the decarboxylation, condensation of malonyl-CoA and acetyl-CoA has the same carbon yield as the condensation of two acetyl-CoA catalyzed by thiolase. Furthermore, substitution of bifunctional aldehyde/alcohol dehydrogenase (AdhE2) with oxygen-tolerant CoA-acylating aldehyde dehydrognease (PduP) and alcohol dehydrogenase (YqhD) increased the 1-butanol production. While production of alcohols by CoA pathway is the most efficient pathway, it may not be suitable for all organisms under all conditions. The data demonstrate that chain elongation by at the expense of an ATP may be more favorable in cyanobacteria.

The disclosure provides a recombinant photoautotroph or photoheterotroph microorganism that produces 1-butanol wherein the alcohol is produced through a malonyl-CoA dependent pathway. In one embodiment, the organism comprises expression or elevated expression of an enzyme that converts acetyl-CoA to malonyl-CoA, malonyl-CoA to Acetoacetyl-CoA, and at least one enzyme that converts (a) acetoacetyl-CoA to (R)- or (S)-3-hydroxybutyryl-CoA and (R)- or (S)-3-hydroxybutyryl-CoA to crotonyl-CoA, crotonyl-CoA to butyryl-CoA, butyryl-CoA to butyraldehyde and butyraldehyde to 1-butanol. In another embodiment of either of the foregoing, the microorganism comprises a metabolic pathway for the production of 1-butanol that is an NADPH dependent pathway. In yet another embodiment, the photoautotrophic or photoheterotrophic microorganism is engineered to express or overexpress one or more polypeptides that convert acetyl-CoA to malonyl-CoA and malonyl-CoA to Acetoacetyl-CoA. In a further embodiment, the one or more polypeptides comprises a nphT7 polypeptide comprising at least 90% identity to SEQ ID NO:18 and having acetoacetyl-CoA synthase activity. In yet another embodiment of any of the foregoing the recombinant microorganism is engineered to express an acetyl-CoA carboxylase. In a further embodiment, the acetyl-CoA carboxylase comprises a sequence that is at least 90% identical to SEQ ID NO:2. In yet another embodiment of any of the foregoing the microorganism further expresses or overexpresses one or more enzymes that carries out a metabolic function selected from the group consisting of (a) converting acetoacetyl-CoA to (R)-3-hydroxybutyryl-CoA, (b) converting acetoacetyl-CoA to (S)-3-hydroxybutyryl-CoA, (c) converting (R)-3-hydroxybutyryl-CoA to crotonyl-CoA, (d) converting (S)-3-hydroxybutyryl-CoA to crotonyl-CoA, (e) converting crotonyl-CoA to butyryl-CoA, (f₁) converting butyryl-CoA to butyraldehyde and butyraldehyde to 1-butanol, or (f₂) butyrl-CoA to 1-butanol. In a further embodiment, the recombinant microorganism comprises an NADPH dependent metabolic pathway that converts (i) acetyl-CoA to malonyl-CoA, (ii) malonyl-CoA to acetoacetyl-CoA, (iii) acetoacetyl-CoA to (R)-3-hydroxybutyryl-CoA, (iv) (R)-3-hydroxybutyryl-CoA to crotonyl-CoA, (v) crotonyl-CoA to butyryl-CoA, (vi) butyryl-CoA to butyraldehyde, and (vii) butyraldehyde to 1-butanol. In another embodiment, the recombinant microorganism comprises a NADH dependent metabolic pathway that converts (i) acetyl-CoA to malonyl-CoA, (ii) malonyl-CoA to acetoacetyl-CoA, (iii) acetoacetyl-CoA to (S)-3-hydroxybutyryl-CoA, (iv) (S)-3-hydroxybutyryl-CoA to crotonyl-CoA, (v) crotonyl-CoA to butyryl-CoA, and (vi) butyryl-CoA to 1-butanol. In yet another embodiment, the recombinant microorganism comprises an NADPH dependent metabolic pathway that converts (i) acetyl-CoA to acetoacetyl-CoA, (ii) acetoacetyl-CoA to (R)-3-hydroxybutyryl-CoA, (iii) (R)-3-hydroxybutyryl-CoA to crotonyl-CoA, (iv) crotonyl-CoA to butyryl-CoA, (v) butyryl-CoA to butyraldehyde, and (vi) butyraldehyde to 1-butanol. In yet another embodiment, the microorganism is a photoautotrophic or photoheterotrophic organism that is engineered to express or overexpress an acetyl-CoA carboxylase and an acetoacetyl-CoA synthase. In another embodiment, the microorganism further expresses or overexpresses one or more enzymes selected from the group consisting of (a) acetoacetyl-CoA reductase, (b) enoyl-CoA hydratase, (c) crotonyl-CoA reductase, and (d) an alcohol/aldehyde dehydrogenase. In another embodiment, the microorganism comprises a photoautotrophic or photoheterotrophic organism that is engineered to express or overexpress an acetyl-CoA carboxylase and an acetoacetyl-CoA synthase and further expresses or overexpresses one or more enzymes selected from the group consisting of (a) acetoacetyl-CoA reductase, (b) enoyl-CoA hydratase, (c) trans-2-enoyl-CoA reductase, and (d) an alcohol/aldehyde dehydrogenase. In another embodiment, the microorganism comprises a photoautotrophic or photoheterotrophic organism that is engineered to express or overexpress an acetyl-CoA carboxylase and an acetoacetyl-CoA synthase and further expresses or overexpresses one or more enzymes selected from the group consisting of (a) acetoacetyl-CoA reductase, (b) enoyl-CoA hydratase, (c) trans-2-enoyl-CoA reductase, and (d) propion- or butyraldehyde dehydrogenase and 1,3-propanediol dehydrogenase. In another embodiment, the microorganism is a photoautotrophic or photoheterotrophic organism and wherein is engineered to express or overexpress an acetyl-CoA carboxylase and an acetoacetyl-CoA synthase and one or more enzymes selected from the group consisting of (a) hydroxybutyryl CoA dehydrogenase, (b) crotonase, (c) crotonyl-CoA reductase, (d) oxygen-tolerant CoA-acylating aldehyde dehydrognease and (e) an alcohol/aldehyde dehydrogenase. In another embodiment, the microorganism comprises a photoautotrophic or photoheterotrophic organism that is engineered to express or overexpress an acetyl-CoA carboxylase and an acetoacetyl-CoA synthase and one or more enzymes selected from the group consisting of (a) hydroxybutyryl CoA dehydrogenase, (b) crotonase, (c) trans-2-enoyl-CoA reductase, (d) oxygen-tolerant CoA-acylating aldehyde dehydrognease and (e) an alcohol/aldehyde dehydrogenase. In yet another embodiment, the microorganism comprises a photoautotrophic or photoheterotrophic organism that is engineered to express or overexpress an acetyl-CoA carboxylase and an acetoacetyl-CoA synthase and one or more enzymes selected from the group consisting of (a) hydroxybutyryl CoA dehydrogenase, (b) crotonase, (c) trans-2-enoyl-CoA reductase, and (d) oxygen-tolerant CoA-acylating aldehyde dehydrognease. In one embodiment, the microorganism comprises a photoautotrophic or photoheterotrophic organism and includes the expression of at least one heterologous, or the over expression of at least one endogenous, target enzyme from the group consisting of an enzyme that converts (i) acetyl-CoA to malonyl-CoA, (ii) malonyl-CoA to Acetoacetyl-CoA, (iii) acetoacetyl-CoA to (R)- or (S)-3-hydroxybutyryl-CoA, (iv) (R)- or (S)-3-hydroxybutyryl-CoA to crotonyl-CoA, (v) crotonyl-CoA to butyryl-CoA, (vi) butyryl-CoA to butyraldehyde and (vi) butyraldehyde to 1-butanol. In another embodiment of any of the foregoing, the microorganism comprises a photoautotrophic or photoheterotrophic organism that comprises a reduction, disruption or knockout of at least one gene encoding an enzyme that competes with a metabolite necessary for the production of a desired higher alcohol product or which produces an unwanted product. In one embodiment, the microorganism comprises a photoautotrophic or photoheterotrophic organism that is engineered to disrupt, delete or knockout one or more genes encoding a polypeptide or protein selected from the group consisting of: (i) an enzyme that catalyzes the NADH-dependent conversion of pyruvate to D-lactate (e.g., ldhA); (ii) an enzyme that promotes catalysis of fumarate and succinate interconversion (e.g., frdBC); (iii) an oxygen transcription regulator; and (iv) an enzyme that catalyzes the conversion of acetyl-coA to acetyl-phosphate (e.g., pta). In a further embodiment, a disruption, deletion or knockout of a combination of an alcohol/acetoaldehyde dehydrogenase and one or more of (i)-(iv). In another embodiment of any of the foregoing the microorganism is recombinantly engineered to express one or more subunits of acetyl-coA carboxylase (AccABCD) that converts acetyl-CoA to malonyl-CoA. In yet another embodiment of any of the foregoing the microorganism is engineered to express of over express one or more genes selected from the group consisting of nphT7, phaB, phaJ, ter, PduP, and yqhD, and wherein the microorganism produces 1-butanol. In another embodiment, the microorganism is engineered to express of over express AccABCD. In a further embodiment, the microorganism expresses a polypeptide having at least 90-100% identity to SEQ ID NO:2 (AccABCD). In yet another embodiment, the microorganism expresses a polypeptide having at least 90-100% identity to SEQ ID NO:18 (nphT7). In yet another embodiment, the microorganism expresses a polypeptide having at least 90-100% identity to SEQ ID NO:30 (phaB). In yet another embodiment, the microorganism expresses a polypeptide having at least 90-100% identity to SEQ ID NO:28 (phaJ). In yet another embodiment, the microorganism expresses a polypeptide having at least 90-100% identity to SEQ ID NO:23, 24, 25, or 26 (ter). In yet another embodiment, the microorganism expresses a polypeptide having at least 90-100% identity to SEQ ID NO:34 (PduP). In yet another embodiment, the microorganism expresses a polypeptide having at least 90-100% identity to SEQ ID NO:32 (yqhD). In yet another embodiment, the microorganism comprises an expression profile selected from the group consisting of:

(a) AccABCD, nphT7, PhaB, PhaJ, Ter, PduP, and YqhD;

(b) nphT7, PhaB, PhaJ, Ter, PduP, and YqhD;

(c) AccABCD, nphT7, PhaB, PhaJ, ccr, PduP, and YqhD;

(d) nphT7, PhaB, PhaJ, ccr, PduP, and YqhD;

(e) AccABCD, nphT7, hbd, crt, Ter, PduP, and YqhD; and

(f) nphT7, hbd, crt, Ter, PduP, and YqhD.

The disclosure also provides a method for producing an alcohol, such as 1-butanol, the method comprising providing a recombinant photoautotroph or photoheterotrophic microorganism of any of the foregoing, culturing the microorganism(s) in the presence of CO₂ under conditions suitable for the conversion of the substrate to an alcohol; and purifying the alcohol.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the disclosure and, together with the detailed description, serve to explain the principles and implementations of the invention.

FIG. 1A-D is a schematic of n-butanol production from CO₂. Schematics for A) Cyanobacteria metabolism, B) Chemical derivatives of n-butanol, C) Native Clostridium acetobutylicum pathway for n-butanol production, and D) Synthetic n-butanol biosynthesis pathway designed with ATP driving force and oxygen tolerance. Abbreviations: Thl, thiolase; Hbd, 3-hydroxybutyryl-CoA dehydrogenase; crt, crotonase; Bcd/EtfAB, butyryl-CoA dehydrogenase electron transferring protein complex; AdhE2, bifunctional aldehyde/alcohol dehydrogenase; AccABCD, acetyl-CoA carboxylase; NphT7, acetoacetyl-CoA synthase; PhaB, acetoacetyl-CoA reductase; PhaJ, R-specific crotonase; Ter, trans-enoyl-CoA reductase; PduP, CoA-acylating propionaldehyde dehydrogenase; YqhD, NADPH dependent alcohol dehydrogenase; G3P, glyceraldehyde-3-phosphate; Pyr, pyruvate; Fdₒₓ, oxidized ferredoxin; Fd_{red}, reduced ferredoxin. * indicates oxygen sensitivity.

FIG. 2A-C shows anaerobic growth rescue of E. coli strain JCL166 by overexpression of Clostridium butanol pathway with different aldehyde dehydrogenases, PduP. A) Schematics of anaerobic growth selection by E. coli strain JCL166. Genes for mixed acid fermentation (adhE, ldhA, frdB) were knocked out. As a result, E. coli strain JCL166 cannot recycle NADH back to NAD+. Cell growth of strain JCL166 is inhibited until a fermentation pathway consuming NADH is engineered into JCL166, establishing the basis of this anaerobic growth selection. B) Growth rescue of strain JCL166 by overexpression of different PduP with Clostridium butanol pathway. C) Alcohol production from the growth rescue broth.

FIG. 3A-B Substrate chain length specificity of CoA-acylating aldehyde dehydrogenases, PduP. A) Specific activity of different PduP (pmol min⁻¹ mg⁻¹). B) Relative activity of PduP. Specific activity was determined with substrate concentration of 1 mM acyl-CoA, 500 uM NADH, 1 mM DTT in 50 mM potassium phosphate buffer at pH 7.15 at 30° C.

FIG. 4A-D shows ethanol biosynthesis from acetyl-CoA. A) Schematics of gene recombination of pduP and yqhD into neutral site I of S. elongatus PCC7942. B) Schematics of ethanol biosynthesis from acetyl-CoA using PduP and YqhD. C) Time course of cumulative ethanol production. Here cumulative titer takes into account the dilutions made to cyanobacteria culture by feeding. D) Cell density of S. elongatus strains ETOH-KP, ETOH-LB, ETOH-LM, and ETOH-SEt.

FIG. 5A-E shows n-butanol production by recombinant S. elongatus. A) Schematics of gene recombination into chromosomes of S. elongatus 7942. B) Time course of observed in-flask n-butanol titer by strains BUOH-LB, BUOH-SE, and BUOH-LM. C) Cell density. D) Daily productivity of n-butanol by S. elongatus strain BUOH-SE. E) Time course of in-flask and cumulative n-butanol produced by strain BUOH-SE. Here cumulative titer takes into account the dilutions made to cyanobacteria culture by feeding.

FIG. 6A-B shows A) Comparison of molar productivities and carbon molar productivities of various acetyl-CoA based compounds produced by cyanobacteria. n-Butanol, fatty acid, 3-hydroxybutyrate, acetone, fatty alcohol. Carbon molar productivity accounts for difference in number of carbons of the products and is calculated by multiplying molar productivity of the target compounds by its numbers of carbons. B) n-Butanol toxicity to S. elongatus strain BUOH-SE.

FIG. 7 shows additional Oxygen tolerant Coenzyme A-acylating aldehyde dehydrogenase. Substrate specificity of additional various CoA-acylating aldehyde dehydrogenases. Assay was conducted using purified aldehyde dehydrogenases.

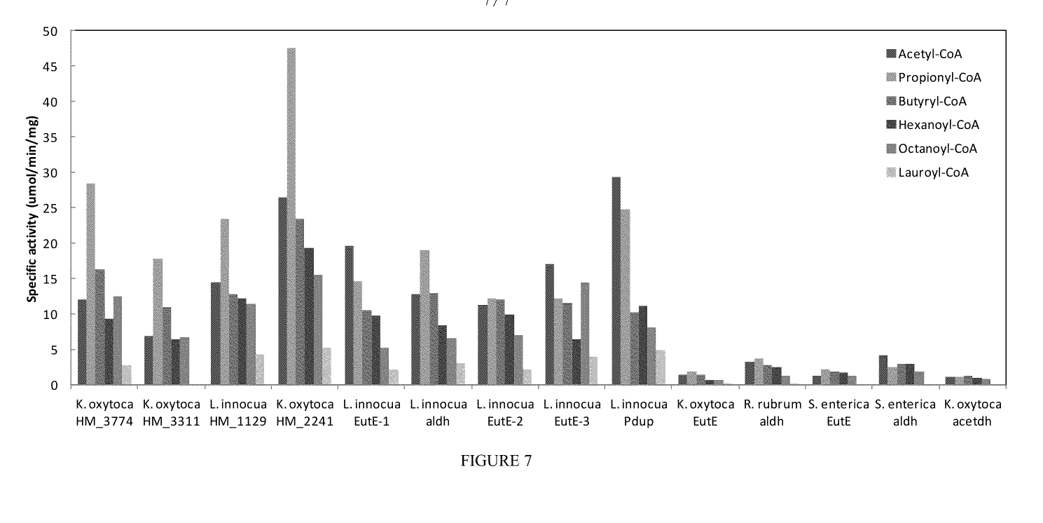

### DETAILED DESCRIPTION

As used herein and in the appended claims, the singular forms “a,” “and,” and “the” include plural referents unless the context clearly dictates otherwise. Thus, for example, reference to “a microorganism” includes a plurality of such microorganisms and reference to “the polypeptide” includes reference to one or more polypeptides and equivalents thereof, and so forth.

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

All publications mentioned herein are incorporated herein by reference in full for the purpose of describing and disclosing the methodologies, which are described in the publications, which might be used in connection with the description herein. The publications discussed above and throughout the text are provided solely for their disclosure prior to the filing date of the present application. Nothing herein is to be construed as an admission that the inventors are not entitled to antedate such disclosure by virtue of prior disclosure.

The disclosure provides accession numbers for various genes, homologs and variants useful in the generation of recombinant microorganism described herein. It is to be understood that homologs and variants described herein are exemplary and non-limiting. Additional homologs, variants and sequences are available to those of skill in the art using various databases including, for example, the National Center for Biotechnology Information (NCBI) access to which is available on the World-Wide-Web.

Butanol is hydrophobic and less volatile than ethanol. 1-Butanol has an energy density closer to gasoline. Butanol at 85 percent strength can be used in cars without any change to the engine (unlike ethanol) and it produces more power than ethanol and almost as much power as gasoline. Butanol is also used as a solvent in chemical and textile processes, organic synthesis and as a chemical intermediate. Butanol also is used as a component of hydraulic and brake fluids and as a base for perfumes.

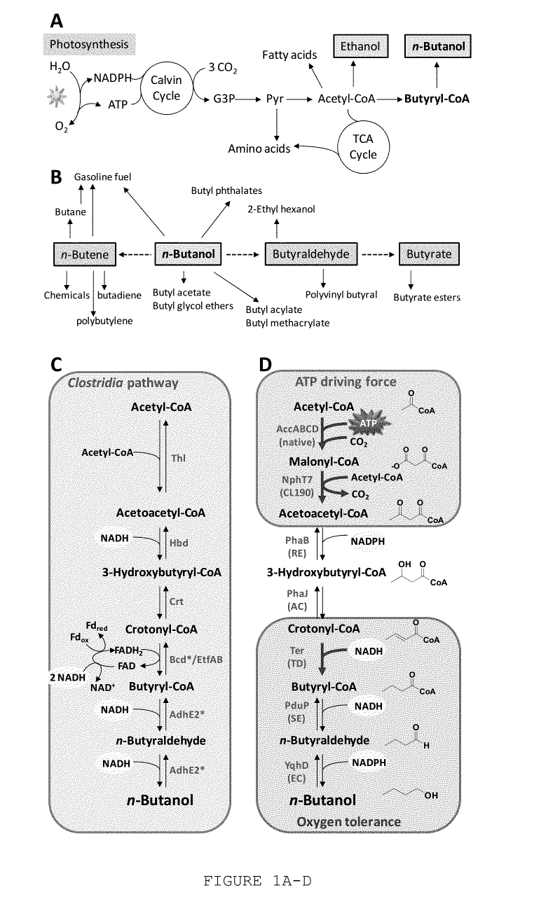

n-Butanol is an important chemical feedstock (FIG. 1B) used to produce solvents (butyl acetate, butyl glycol ethers), polymers (butyl acrylate, butyl methacrylate), and plasticizers (butyl phthalate, butylbenzyl phthalate). Derivatives of n-butanol including n-butene, butyraldehyde, and butyrate are also used to synthesize a wide array of chemical products. Furthermore n-butanol is a more efficient fuel additive or substitute than ethanol as it is higher in energy density and lower in hygroscopicity, allowing it to be compatible with existing infrastructure. Currently, global n-butanol consumption is 2.9 million metric tons, representing a $5.7 billion market, and continues to grow 4.7% a year.

Biological production of chemical and fuel is an attractive direction towards a sustainable future. In particular, 1-butanol has received increasing attention as it is a potential fuel substitute and a chemical feedstock. 1-Butanol can be produced by two distinctive pathways: 2-ketoacid pathway and Coenzyme A (CoA) dependent pathway. The 2-ketoacid pathway utilizes either threonine synthetic pathway or citramalate pathway for producing 2-ketobutyrate. Leucine biosynthesis then elongates 2-ketobutyrate into 2-ketovalarate. 2-Ketovalarate is then decarboxylated and reduced into 1-butanol. On the other hand, the CoA pathway follows the chemistry of β-oxidation in reverse. Acetyl-CoA is condensed into acetoacetyl-CoA which is then further reduced to 1-butanol. Furthermore, using this reversed β-oxidation, 1-butanol can be elongated to 1-hexanol and other long even-numbered chain primary alcohols. A comparison of these 1-butanol synthesis pathways reveals that CoA pathway is the most carbon energy efficient pathway for producing 1-butanol. Citramalate pathway requires an additional acetyl-CoA and threonine pathway requires two ATP.

The CoA pathway is a natural fermentation pathway used by Clostridium species. However CoA pathway is not expressed well in recombinant chemoheterotrophs, resulting in low titer 1-butanol production ranging from 2.5 mg/L to 1,200 mg/L with sugar as the substrate. The hypothesized limiting step is the reduction of crotonyl-CoA by the butyryl-CoA dehydrogenase/electron transferring flavoprotein (Bcd/EtfAB) complex. Bcd/EtfAB complex is difficult to use in recombinant systems because of its poor expression, instability, and potential requirement for ferredoxin. This problem was overcome by replacing Bcd/EtfAB complex with trans-2-enoyl-CoA reductase (Ter). Ter expresses well and directly reduces crotonyl-CoA with NADH. This modified CoA 1-butanol pathway is catalyzed by: thiolase (AtoB), 3-hydroxybutyryl-CoA dehydrogenase (Hbd), crotonase (Crt), Ter, and bifunctional aldehyde/alcohol dehydrogenase (AdhE2). In combination of expressing these enzymes and engineering NADH and acetyl-CoA accumulation as driving forces, successful recombinant 1-butanol production has been demonstrated in E. coli with high titer (15-30 g/L) and yield (70%-88% of theoretical). This result demonstrated the efficiency of the CoA pathway for 1-butanol fermentation.

Microbial n-butanol is produced via metabolic pathways in selected species of Clostridia and in recombinant organisms via synthetic pathways. The Clostridium Coenzyme A (CoA) dependent pathway is of particular interest for its efficient chain elongation which can be engineered to synthesize longer chain chemicals. The native Clostridium pathway (FIG. 1C) proceeds by condensation of two acetyl-CoA and follows a series of reduction using NADH and dehydration to form n-butanol. As mentioned above, the enzyme butyryl-CoA dehydrogenase complex (Bcd-EtfAB) has been difficult to express in heterologous organisms due to oxygen sensitivity and potential requirement for accessory redox partners which may not exist in heterologous organisms (FIG. 1C). Expression of a trans-2-enoyl-CoA reductase (Ter) using NADH for direct reduction of crotonyl-CoA efficiently avoided the need of Bcd/etfAB complex and achieved high flux production of n-butanol production in Escherichia coli.

Cyanobacteria are currently being developed as microbial factories for production of renewable chemicals by redirecting carbon fluxes from central metabolism into desirable products (FIG. 1A). However, metabolic engineering of cyanobacteria has been challenging because of their incompletely characterized physiology, limited genetic tools, and relatively low growth rate. In particular, pathways originated from anaerobes are often difficult to express in cyanobacteria due to enzyme oxygen sensitivity and difference in redox environment.

As mentioned above, the success of the CoA-dependent pathway in E. coli was not as effective in photoautotrophs. By expressing the same enzymes in cyanobacteria Synechococcus elongatus PCC 7942, photosynthetic 1-butanol production from CO₂ was substantially lower. 1-Butanol production was achieved by this strain when internal carbon storage made by CO₂ fixation in light conditions was fermented under anoxic conditions.

This could be due to the fact that Acetyl-CoA is the precursor for fermentation pathway and the TCA cycle, both of which are not active in light conditions. Furthermore, photosynthesis generates NADPH, but not NADH, and the interconversion between the two may not be efficient. Without a significant driving force against the unfavorable thermodynamic gradient, 1-butanol production cannot reach the levels seen in other organisms. The difficulty of direct photosynthetic production of 1-butanol is in sharp contrast to the production of isobutanol (450 mg/L) and isobutyraldehyde (100 mg/L) by S. elongatus PCC 7942, which has an irreversible decarboxylation step as the first committed reaction to drive the flux toward the products.

The Ter-dependent pathway in cyanobacterium Synechococcus elongatus PCC 7942 requires anaerobic treatment and inhibition of photosystem II to avoid oxygen evolution for the synthesis of n-butanol. Under these conditions, n-butanol production relies on internal storage of carbon. In contrast, isobutanol was produced in relatively high titer and productivity. Comparing the two pathways, the disclosure identified that the first step in the isobutanol pathway evolves CO₂, which provides a driving force, and that the isobutanol pathway does not involve any oxygen-sensitive enzymes. Thus, the difficulty of photosynthetic n-butanol production may be attributable to the lack of thermodynamic driving forces and oxygen sensitivity of pathway enzymes. To solve the first problem, an ATP driving force was engineered into a recombinant microorganism of the disclosure (FIG. 1D, top box) inspired by fatty acid biosynthesis and enabled the first demonstration of direct photosynthetic production of n-butanol. Activation of acetyl-CoA into malonyl-CoA with ATP provides the thermodynamic driving force necessary to overcome the energetic barrier of acetyl-CoA condensation. Utilizing this malonyl-CoA dependent pathway, the resulting S. elongatus strain produced 30 mg/L of n-butanol with productivity of about 1-2 mg/L/d, typically about 2 mg/L/d.

Instead of using an excess of acetyl-CoA to drive production, the disclosure demonstrates that ATP can be used to drive the thermodynamically unfavorable condensation of two acetyl-coA molecules under photosynthetic conditions. Thus, the disclosure engineers into a microorganism the ATP-driven malonyl-CoA synthesis and decarboxylative carbon chain elongation used in fatty acid synthesis to drive the carbon flux into the formation of acetoacetyl-CoA, which then undergoes the reverse β-oxidation to synthesize 1-butanol. In addition, to further optimize that synthesis the subsequent NADH-dependent enzymes were replaced with NADPH-dependent enzymes to achieve 1-butanol synthesis under photosynthetic conditions.

In theory, the excess ATP consumption in the cell may cause a decrease in biomass. Thus, with notable exceptions, most metabolic engineering designs do not choose to increase ATP consumption. Although many natural examples of microbes using ATP to drive reactions, most of them are highly regulated. Therefore, the results presented herein are unexpected.

In view of the foregoing, the disclosure provides organisms comprising metabolically engineered biosynthetic pathways that utilize an organism's CoA pathway with increased ATP consumption to drive biofuel and chemical production. The biofuel and chemical production utilizing the organism's CoA pathway offers several advantages. Not only does it avoid the difficulty of expressing a large set of foreign genes but it also minimizes the possible accumulation of toxic intermediates. Furthermore, the disclosure demonstrates that by reducing oxidation of NADH by competitive pathways, effective n-butanol production and/or coupling NADH utilization more closely to the n-butanol production pathway described herein provides an increase in n-butanol production. Identifying competing (oxidative) pathways in various organism is within the skill in the art and various enzymes in such pathways can be reduced by knocking out the polynucleotide encoding such enzyme or reducing expression. Accordingly, exemplary genes and sequences are provided herein, however, one will recognize the ability to identify homologs in various species as well as enzymes having similar synthetic or catabolic activity based on the teachings herein.

CoA-acylating butyraldehyde dehydrogenase (Bldh), which catalyzes the termination of CoA-dependent chain elongation by reducing butyryl-CoA into butyraldehyde, is an oxygen-sensitive enzyme which was hypothesized to limit the productivity of the pathway. Bldh from Clostridium beijerinckii has been characterized to lose activity upon exposure to oxygen. Similarly, AdhE2, a bifunctional aldehyde/alcohol dehydrogenase from traditional n-butanol producer Clostridium acetobutylicum, is also oxygen sensitive.

This disclosure addresses the oxygen sensitivity of Bldh by utilizing a CoA-acylating propionaldehyde dehydrogenase (PduP) from coenzyme-B12-dependent 1,2-propanediol degradation pathway to substitute for Bldh under oxygenic photosynthetic conditions. PduP from Salmonella enterica has been shown to catalyze propionaldehyde oxidation to propionyl-CoA for detoxification of propionaldehyde, an intermediate formed from 1,2-propanediol degradation. It was reasoned that since 1,2-propanediol degradation occurs under aerobic conditions, PduP is likely to be oxygen tolerant. As described below six different PduP homologues were cloned and purified with poly-His tags, and their kinetic properties were determined. Other PduP homologues are known and are identified herein. These PduP homologues catalyze reversible reduction of acyl-CoA of different carbon chain lengths. Expression of PduP with the enzymes of the n-butanol synthesis pathway in S. elongatus resulted in autotrophic n-butanol production to a cumulative titer of about 400 mg/L with peak productivity of about 51 mg/L/d, exceeding the strain expressing Bldh by 20 fold. These results demonstrate that oxygen tolerance is an important factor for alcohol production under photosynthetic conditions. The disclosure thus provide in one embodiment, a recombinant microorganism that heterologously expresses a PduP enzyme or homolog thereof and produces an alcohol (e.g., n-butanol, ethanol and the like). In one embodiment, the microorganism produces n-butanol in excess of 5 mg/L/d to about 51 mg/L/d (e.g., greater than 10 mg/L/d, greater than 15 mg/L/d, greater than 20 mg/L/d, greater than 30 mg/L/d, greater than 40 mg/L/d, greater than 45 mg/L/d and greater than 51 mg/L/d, but less than about 55 mg/L/d).

The disclosure provides methods and compositions for the production of biofuel alcohols (e.g., butanol, isobutanol, ethanol etc.). In one embodiment, the disclosure provides methods and compositions for the production of n-butanol, butyraldehyde, butyrate, butane and derivates, metabolites and chemical conversions of any of the foregoing including, but not limited to butane, butadiene, polybutylene, butyl acetate, butyl glycol ethers, butyl phthalates, 2-ethyl hexanol, polyvinyl butyral, and butyrate esters using a culture of microorganisms. In one embodiment, the culture utilizes utilizes CO₂ as a carbon source. Examples of such microorganisms that utilize CO₂ as a carbon source include photoautotrophs. In some embodiments the methods and compositions comprise a co-culture of photoautotrophs and a photoheterotroph or a photoautotroph and a microorganism that cannot utilize CO₂ as a carbon source.

The disclosure provides microorganisms that comprise an artificially engineered ATP consumption pathway to produce, for example, malonyl-CoA, which can be metabolized into biofuels and other chemicals. The disclosure shows that artificially engineered ATP consumption through a pathway modification can drive the reaction of acetyl-CoA to acetoacetyl-CoA (a thermodynamically unfavorable reaction) forward and enables for the direct photosynthetic production of n-butanol and other chemicals and biofuels from photoautotrophs such as the cyanobacteria Synechococcus elongates PCC 7942. In addition, the disclosure demonstrates that substitution of bifunctional aldehyde/alcohol dehydrogenase (AdhE2) with separate oxygen tolerant CoA-acylating aldehyde dehydrogenases (PduP) (or homologs) and NADPH-dependent alcohol dehydrogenase (YqhD) increased biofuel production (e.g., n-butanol production).

As used herein, the term “metabolically engineered” or “metabolic engineering” involves rational pathway design and assembly of biosynthetic genes, genes associated with operons, and control elements of such polynucleotides, for the production of a desired metabolite, such as an acetoacetyl-CoA, metabolites in the production of biofuels, including n-butanol or metabolic products derived therefrom, in a microorganism. “Metabolically engineered” can further include optimization of metabolic flux by regulation and optimization of transcription, translation, protein stability, reducing agents and protein functionality using genetic engineering and appropriate culture condition including the reduction of, disruption, or knocking out of, a competing metabolic pathway that competes with an intermediate or use of a cofactor or energy source, leading to a desired pathway. A biosynthetic gene can be heterologous to the host microorganism, either by virtue of being foreign to the host, or being modified by mutagenesis, recombination, and/or association with a heterologous expression control sequence in an endogenous host cell. In one embodiment, where the polynucleotide is xenogenetic to the host organism, the polynucleotide can be codon optimized.

The term “biosynthetic pathway”, also referred to as “metabolic pathway”, refers to a set of anabolic or catabolic biochemical reactions for converting (transmuting) one chemical species into another. Gene products belong to the same “metabolic pathway” if they, in parallel or in series, act on the same substrate, produce the same product, or act on or produce a metabolic intermediate (i.e., metabolite) between the same substrate and metabolite end product.

The term “substrate” or “suitable substrate” refers to any substance or compound that is converted or meant to be converted into another compound by the action of an enzyme. The term includes not only a single compound, but also combinations of compounds, such as solutions, mixtures and other materials which contain at least one substrate, or derivatives thereof. Further, the term “substrate” encompasses not only compounds that provide a carbon source suitable for use as a starting material, such as CO₂, or any biomass derived sugar, but also intermediate and end product metabolites used in a pathway associated with a metabolically engineered microorganism as described herein.

The term “1-butanol” or “n-butanol” generally refers to a straight chain isomer with the alcohol functional group at the terminal carbon. The straight chain isomer with the alcohol at an internal carbon is sec-butanol or 2-butanol. The branched isomer with the alcohol at a terminal carbon is isobutanol, and the branched isomer with the alcohol at the internal carbon is tert-butanol.

Recombinant microorganisms provided herein can express a plurality of target enzymes involved in metabolic pathway(s) for the production of a biofuel, such as n-butanol, from a suitable carbon substrate. In one embodiment, the microorganism expresses enzymes that convert Acetyl-CoA to n-butanol, wherein the microorganism expresses a heterologous coenzyme-A-acylating propionaldehyde dehydrogenase, expresses a coenzvme-A-acylatinq propionaldehyde dehydrogenase using a heterologous promoter attached to an endogenous coenzyme-A-acylating propionaldehyde dehydrogenase nucleic acid, or over expresses a coenzyme-A-acylating propionaldehyde dehydrogenase.

Accordingly, metabolically “engineered” or “modified” microorganisms are produced via the introduction of genetic material into a host or parental microorganism of choice thereby modifying or altering the cellular physiology and biochemistry of the microorganism. Through the introduction of genetic material the parental microorganism acquires new properties, e.g. the ability to produce a new, or greater quantities of, a metabolite. In an illustrative embodiment, the introduction of genetic material into a parental microorganism results in a new or modified ability to produce n-butanol, other desirable chemicals derived from n-butanol, or precursor metabolites of n-butanol. The genetic material introduced into the parental microorganism contains gene(s), or parts of genes, coding for one or more of the enzymes involved in a biosynthetic pathway for the production of an alcohol or chemical (e.g., 1-butanol) and may also include additional elements for the expression and/or regulation of expression of these genes, e.g. promoter sequences.

In general, the recombinant microorganisms comprises at least one recombinant metabolic pathway that comprises a target enzyme and may further include a reduction in activity or reduction in expression of an enzyme in a competitive biosynthetic pathway. The pathway acts to modify a substrate or metabolic intermediate in the production of an alcohol such as 1-butanol. The target enzyme is encoded by, and expressed from, a polynucleotide derived from a suitable biological source. In some embodiments, the polynucleotide comprises a gene derived from a bacterial or yeast source and recombinantly engineered into a microorganism of the disclosure. In one embodiment, the disclosure provides a recombinant microorganism comprising elevated expression of at least one target enzyme as compared to a parental microorganism or encodes an enzyme not found in the parental organism. For example, in one embodiment, a photoautotrophic or photoheterotrophic organism is engineered to express or overexpress one or more polypeptides that convert acetyl-CoA to Malonyl-CoA and malonyl-CoA to Acetoacetyl-CoA. For example, in one embodiment, the recombinant microorganism is engineered to express or overexpress an acetyl-CoA carboxylase such as Acc. In a further embodiment, the microorganism is engineered to express or over express an acetoacetyl-CoA synthase, such as NphT7, to form acetoacetyl-CoA. In yet a further embodiment, the microorganism further expresses or overexpresses one or more enzymes that carries out a metabolic function selected from the group consisting of (a) converting acetoacetyl-CoA to (R)-3-hydroxybutyryl-CoA, (b) converting acetoacetyl-CoA to (S)-3-hydroxybutyryl-CoA, (c) converting (R)-3-hydroxybutyryl-CoA to crotonyl-CoA, (d) converting (S)-3-hydroxybutyryl-CoA to crotonyl-CoA, (e) converting crotonyl-CoA to butyryl-CoA, (f₁) converting butyryl-CoA to butyraldehyde and butyraldehyde to 1-butanol, or (f₂) butyrl-CoA to 1-butanol. In one embodiment, the recombinant microorganism comprises an NADPH dependent metabolic pathway that converts acetyl-CoA to n-butanol that includes the conversion of (i) acetyl-CoA to malonyl-CoA, (ii) malonyl-CoA to acetoacetyl-CoA, (iii) acetoacetyl-CoA to (R)-3-hydroxybutyryl-CoA, (iv) (R)-3-hydroxybutyryl-CoA to crotonyl-CoA, (v) crotonyl-CoA to butyryl-CoA, (vi) butyryl-CoA to butyraldehyde, and (vii) butyraldehyde to n-butanol. In another embodiment, the recombinant microorganism comprises a NADH dependent metabolic pathway that converts (i) acetyl-CoA to malonyl-CoA, (ii) malonyl-CoA to acetoacetyl-CoA, (iii) acetoacetyl-CoA to (S)-3-hydroxybutyryl-CoA, (iv) (S)-3-hydroxybutyryl-CoA to crotonyl-CoA, (v) crotonyl-CoA to butyryl-CoA, and (vi) butyryl-CoA to 1-butanol. In yet another embodiment, the recombinant microorganism comprises an NADPH dependent metabolic pathway that converts (i) acetyl-CoA to acetoacetyl-CoA, (ii) acetoacetyl-CoA to (R)-3-hydroxybutyryl-CoA, (iii) (R)-3-hydroxybutyryl-CoA to crotonyl-CoA, (iv) crotonyl-CoA to butyryl-CoA, (v) butyryl-CoA to butyraldehyde, and (vi) butyraldehyde to n-butanol.

For example, in one embodiment, the microorganism comprises a photoautotrophic or photoheterotrophic organism that is engineered to express or overexpress an acetyl-CoA carboxylase and an acetoacetyl-CoA synthase and further expresses or overexpresses one or more enzymes selected from the group consisting of (a) acetoacetyl-CoA reductase, (b) enoyl-CoA hydratase, (c) trans-2-enoyl-CoA reductase, and (d) a coenzyme-A-acylating propionaldehyde dehydrogenase and 1,3-propanediol dehydrogenase.

In another embodiment, a photoautotrophic or photoheterotrophic organism that is engineered to express or overexpress an acetyl-CoA carboxylase and an acetoacetyl-CoA synthase. In yet a further embodiment, the microorganism further expresses or overexpresses one or more enzymes selected from the group consisting of (a) hydroxybutyryl CoA dehydrogenase, (b) crotonase, (c) crotonyl-CoA reductase, and (d) a coenzyme-A-acylating propionaldehyde dehydrogenase. In another example, the microorganism comprises a photoautotrophic or photoheterotrophic organism that is engineered to express or overexpress an acetyl-CoA carboxylase and an acetoacetyl-CoA synthase and further expresses or overexpresses one or more enzymes selected from the group consisting of (a) hydroxybutyryl CoA dehydrogenase, (b) crotonase, (c) trans-2-enoyl-CoA reductase, and (d) a coenzyme-A-acylating propionaldehyde dehydrogenase and an alcohol/aldehyde dehydrogenase. In another example, the microorganism comprises a photoautotrophic or photoheterotrophic organism that is engineered to express or overexpress an acetyl-CoA carboxylase and an acetoacetyl-CoA synthase and further expresses or overexpresses one or more enzymes selected from the group consisting of (a) hydroxybutyryl CoA dehydrogenase, (b) crotonase, (c) trans-2-enoyl-CoA reductase, and (d) a coenzyme-A-acylating propionaldehyde dehydrogenase and 1,3-propanediol dehydrogenase. In another example, the microorganism comprises a photoautotrophic or photoheterotrophic organism that is engineered to express or overexpress an acetyl-CoA carboxylase and an acetoacetyl-CoA synthase and further expresses or overexpresses a coenzyme-A-acylating propionaldehyde dehydrogenase.

Accordingly, a recombinant microorganism provided herein includes the heterologous or elevated expression of at least one target enzyme such as an enzyme that converts acetyl-CoA to malonyl-CoA, malonyl-CoA to Acetoacetyl-CoA, acetoacetyl-CoA to (R)- or (S)-3-hydroxybutyryl-CoA, (R)- or (S)-3-hydroxybutyryl-CoA to crotonyl-CoA, crotonyl-CoA to butyryl-CoA, butyryl-CoA to butyraldehyde and butyraldehyde to 1-butanol. In other embodiments, a recombinant microorganism can express a plurality of target enzymes involved in pathway to produce n-butanol as depicted in FIG. 1D. The plurality of enzymes can include one or more subunits of acetyl-coA carboxylase (AccABCD, for example accession number AAC73296 AAN73296, EC 6.4.1.2), Acetoacetyl-CoA reductase (phaB, e.g., from R. eutropha) (EC 1.1.1.36) that generates 3-hydroxybutyryl-CoA from acetoacetyl-CoA and NADPH, (R)-specific enoyl-CoA hydratase (PhaJ) derived from, for example, Aeromonas caviae or Pseudomonas aeruginosa (Fukui et al., J. Bacteriol. 180:667, 1998; Tsage et al., FEMS Microbiol. Lett. 184:193, 2000), a coenzyme-A-acylating propionaldehyde dehydrogenase (PduP) or alcohol dehydrogenase (AdhE2), Ter, Ccr, or any combination thereof.

In another or further embodiment, the microorganism comprises a reduction, disruption or knockout of at least one gene encoding an enzyme that competes with a metabolite necessary for the production of a desired chemical product or which produces an unwanted product. For example, the recombinant microorganism may include a disruption, deletion or knockout of expression of an alcohol/acetoaldehyde dehydrogenase that preferentially uses acetyl-coA as a substrate (e.g., adhE gene), as compared to a parental microorganism. Other disruptions, deletions or knockouts can include one or more genes encoding a polypeptide or protein selected from the group consisting of: (i) an enzyme that catalyzes the NADH-dependent conversion of pyruvate to D-lactate (e.g., ldhA); (ii) an enzyme that promotes catalysis of fumarate and succinate interconversion (e.g., frdBC); (iii) an oxygen transcription regulator; and (iv) an enzyme that catalyzes the conversion of acetyl-coA to acetyl-phosphate (e.g., pta). In one embodiment, the microorganism comprises a disruption, deletion or knockout of any combination of an alcohol/acetoaldehyde dehydrogenase and one or more of (i)-(iv) above. Through the reduction, disruption or knocking out of a gene or polynucleotide the microorganism acquires new or improved properties (e.g., the ability to produce a new or greater quantity of an interacellular metabolite, improve the flux of a metabolite down a desired pathway, and/or reduce the production of undesirable by-products).

Microorganisms provided herein are modified to produce metabolites in quantities not available in the parental microorganism. A “metabolite” refers to any substance produced by metabolism or a substance necessary for or taking part in a particular metabolic process. A metabolite can be an organic compound that is a starting material (e.g., glucose or pyruvate), an intermediate (e.g., acetyl-coA) in, or an end product (e.g., n-butanol), of metabolism. Metabolites can be used to construct more complex molecules, or they can be broken down into simpler ones. Intermediate metabolites may be synthesized from other metabolites, perhaps used to make more complex substances, or broken down into simpler compounds, often with the release of chemical energy.

As described above, a recombinant microorganism of the disclosure comprise expression of a heterologous acetyl-CoA carboxylase or elevated expression of an endogenous acetyl-CoA carboxylase. Depending upon the organism used a heterologous acetyl-CoA carboxylase can be engineered for expression in the organism. Alternatively, a native acetyl-CoA carboxylase can be overexpressed. The acetyl-CoA carboxylase (accABCD, EC 6.4.1.2) comprises an operon of multiple subunits, e.g., accA, accB, accC, accD. Acetyl-CoA carboxylase (Acc) is a multisubunit enzyme encoded by four separate genes, accABCD (accA, accB, accC, and accD; see, e.g., accession numbers: NP414727 (SEQ ID NO:35/36), NP417721 (SEQ ID NO:37/38), NP417722 (SEQ ID NO:39/40), NP416819 (SEQ ID NO:41/42), respectively, incorporated herein by reference). As used herein, the term “acetyl-CoA carboxylase carboxytransferase” means the enzyme that catalyzes the carboxylation of acetyl-CoA to malonyl-CoA and forms a tetramer composed of two alpha and two beta subunits. One of the subunits corresponds to the acetyl-CoA carboxylase carboxytransferase subunit alpha, encoded by the accA gene. In one embodiment, the acetyl-CoA carboxylase carboxytransferase subunit alpha expressed from an expression vector in accordance with the disclosure is derived from E. coli or P. aeruginosa and includes homologs thereof. For example, the acetyl-CoA carboxylase carboxytransferase subunit alpha can have the nucleotide sequence of SEQ ID NO:1, encoded by the amino acid sequence of SEQ ID NO: 2 and polypeptides having at least 70%, 80%, 90%, 95%, 98%, or 99% identity thereto and having acetyl-CoA carboxylase activity. A homolog of the acetyl-coA carboxylase carboxytranferase alpha subunit of SEQ ID NO:1 includes the sequence associated with NCBI Reference Accession No. NP414727 (SEQ ID NO:35). In another embodiment, the acetyl-CoA carboxylase is encoded by a vector comprising SEQ ID NOs:35, 37, 39, and 41 operably linked to provide an accABCD polypeptide upon expression.

In one embodiment, any of the microorganisms of the disclosure can comprise one or more heterologous nucleic acid(s) encoding an acetoacetyl-CoA synthase polypeptide. The acetoacetyl-CoA synthase enzyme can be encoded by a gene nphT7. NphT7 is a gene encoding an enzyme having the activity of synthesizing acetoacetyl-CoA from malonyl-CoA and acetyl-CoA and having minimal to no activity synthesizing acetoacetyl-CoA from two acetyl-CoA molecules. An acetoacetyl-CoA synthase gene from an actinomycete of the genus Streptomyces CL190 strain is described in U.S. Patent Application Publication No. 2010/0285549, the disclosure of which is incorporated by reference herein. Acetoacetyl-CoA synthase can also be referred to as acetyl CoA:malonyl CoA acyltransferase. A representative acetoacetyl-CoA synthase (or acetyl CoA:malonyl CoA acyltransferase) that can be used is has a sequence as set forth in Genbank AB540131.1 (SEQ ID NO:17/18).

In one embodiment, acetoacetyl-CoA synthase of the disclosure synthesizes acetoacetyl-CoA from malonyl-CoA and acetyl-CoA via an irreversible reaction. The use of acetoacetyl-CoA synthase to generate acetyl-CoA provides an additional advantage in that this reaction is irreversible while acetoacetyl-CoA thiolase enzyme's action of synthesizing acetoacetyl-CoA from two acetyl-CoA molecules is reversible. Consequently, the use of acetoacetyl-CoA synthase to synthesize acetoacetyl-CoA from malonyl-CoA and acetyl-CoA drives the reaction and production of biofuels and chemicals that use acetoacetyl-CoA as a metabolite forward (e.g., the production of n-butanol).

An example of such an acetoacetyl-CoA synthase is set forth in SEQ ID NO:18. In one embodiment, a polypeptide comprising at least 80% sequence identity to the amino acid sequence of SEQ ID NO: 18 corresponds to an acetoacetyl-CoA synthase capable of producing acetoacetyl-CoA from malonyl-CoA and acetyl-CoA and having little or no activity of synthesizing acetoacetyl-CoA from two acetyl-CoA molecules. In one embodiment, the polynucleotide encoding a polypeptide having the amino acid sequence of SEQ ID NO: 18 can be obtained by a nucleic acid amplification method (e.g., PCR) with the use of genomic DNA obtained from an actinomycete of the Streptomyces sp. CL190 strain. As described herein, an acetoacetyl-CoA synthase is not limited to a polypeptide having the amino acid sequence of SEQ ID NO: 18 from an actinomycete of the Streptomyces sp. CL190 strain. Any polypeptide having the ability to synthesize acetoacetyl-CoA from malonyl-CoA and acetyl-CoA and which can, but preferably does not, synthesize acetoacetyl-CoA from two acetyl-CoA molecules can be used in the presently described methods. In certain embodiments, the acetoacetyl-CoA synthase can have an amino acid sequence that is substantially identical to the amino acid sequence of SEQ ID NO: 18 and having the function of synthesizing acetoacetyl-CoA from malonyl-CoA and acetyl-CoA. For example, the disclosure includes polypeptides having at least about 80% identity, at least about 85%, at least about 90%, at least about 91%, at least about 92%, at least about 93%, at least about 94%, at least about 95%, at least about 96%, at least about 97%, at least about 98%, and at least about 99% identity to SEQ ID NO:18 and having acetoacetyl-CoA synthase activity. In other embodiments, the acetoacetyl-CoA synthase can have an amino acid sequence derived from the amino acid sequence of SEQ ID NO: 18 by substitution, deletion, addition, or insertion of 1 or more amino acid(s) (e.g., 1-10) and having the function of synthesizing acetoacetyl-CoA from malonyl-CoA and acetyl-CoA.

The recombinant microorganism can produce a metabolite that includes a 3-hydroxybutyryl-CoA using an NADH dependent step from a substrate that includes acetoacetyl-CoA. The hydroxybutyryl CoA dehydrogenase can be encoded by an hbd gene or homolog thereof. The hbd gene can be derived from various microorganisms including Clostridium acetobutylicum, Clostridium difficile, Dastricha ruminatium, Butyrivibrio fibrisolvens, Treponema phagedemes, Acidaminococcus fermentans, Clostridium kluyveri, Syntrophospora bryanti, and Thermoanaerobacterium thermosaccharolyticum.

3 hydroxy-butyryl-coA-dehydrogenase catalyzes the conversion of acetoacetyl-coA to 3-hydroxybutyryl-CoA. Depending upon the organism used a heterologous 3-hydroxy-butyryl-coA-dehydrogenase can be engineered for expression in the organism. Alternatively a native 3-hydroxy-butyryl-coA-dehydrogenase can be overexpressed. 3-hydroxy-butyryl-coA-dehydrogenase is encoded in C. acetobuylicum by hbd. HBD homologs and variants are known. For examples, such homologs and variants include, for example, 3-hydroxybutyryl-CoA dehydrogenase (Clostridium acetobutylicum ATCC 824) gi|15895965|ref|NP_349314.1|(15895965); 3-hydroxybutyryl-CoA dehydrogenase (Bordetella pertussis Tohama I) gi|33571103|emb|CAE40597.1|(33571103); 3-hydroxybutyryl-CoA dehydrogenase (Streptomyces coelicolor A3(2)) gi|21223745|ref|NP_629524.1|(21223745); 3-hydroxybutyryl-CoA dehydrogenase gi|1055222|gb|AAA95971.1|(1055222); 3-hydroxybutyryl-CoA dehydrogenase (Clostridium perfringens str. 13) gi|18311280|ref|NP_563214.1|(18311280); 3-hydroxybutyryl-CoA dehydrogenase (Clostridium perfringens str. 13) gi|18145963|dbj|BAB82004.1|(18145963) each sequence associated with the accession number is incorporated herein by reference in its entirety. SEQ ID NO:20 sets forth an exemplary hbd polypeptide sequence. In certain embodiments, the 3 hydroxy-butyryl-coA-dehydrogenase can have an amino acid sequence that is substantially identical to the amino acid sequence of SEQ ID NO: 20 and having 3 hydroxy-butyryl-coA-dehydrogenase activity. For example, the disclosure includes polypeptides having at least about 80% identity, at least about 85%, at least about 90%, at least about 91%, at least about 92%, at least about 93%, at least about 94%, at least about 95%, at least about 96%, at least about 97%, at least about 98%, and at least about 99% identity to SEQ ID NO:20 and having 3 hydroxy-butyryl-coA-dehydrogenase. In other embodiments, the 3 hydroxy-butyryl-coA-dehydrogenase can have an amino acid sequence derived from the amino acid sequence of SEQ ID NO: 20 by substitution, deletion, addition, or insertion of 1 or more amino acid(s) (e.g., 1-10) and having 3 hydroxy-butyryl-coA-dehydrogenase activity.

Crotonase catalyzes the conversion of 3-hydroxybutyryl-CoA to crotonyl-CoA. Depending upon the organism used a heterologous crotonase can be engineered for expression in the organism. Alternatively a native Crotonase can be overexpressed. In embodiments where hbd is used, the organism will typically be engineered to express a crotonase. Crotonase is encoded in C. acetobuylicum by crt. CRT homologs and variants are known. For examples, such homologs and variants include, for example, crotonase (butyrate-producing bacterium L2-50) gi|119370267|gb|ABL68062.1|(119370267); crotonase gi|1055218|gb|AAA95967.1|(1055218); crotonase (Clostridium perfringens NCTC 8239) gi|168218170|ref|ZP_02643795.1|(168218170); crotonase (Clostridium perfringens CPE str. F4969) gi|168215036|ref|ZP_02640661.1|(168215036); crotonase (Clostridium perfringens E str. JGS1987) gi|168207716|ref|ZP_02633721.1|(168207716); crotonase (Azoarcus sp. EbN1) gi|56476648|ref|YP_158237.1|(56476648); crotonase (Roseovarius sp. TM1035) gi|149203066|ref|ZP_01880037.1|(149203066); crotonase (Roseovarius sp. TM1035) gi|149143612|gb|EDM31648.1|(149143612); crotonase; 3-hydroxbutyryl-CoA dehydratase (Mesorhizobium loti MAFF303099) gi|14027492|dbj|BAB53761.1|(14027492); crotonase (Roseobacter sp. SK209-2-6) gi|126738922|ref|ZP_01754618.1|(126738922); crotonase (Roseobacter sp. SK209-2-6) gi|126720103|gb|EBA16810.1|(126720103); crotonase (Marinobacter sp. ELB17) gi|126665001|ref|ZP_01735984.1|(126665001); crotonase (Marinobacter sp. ELB17) gi|126630371|gb|EBA00986.1|(126630371); crotonase (Azoarcus sp. EbN1) gi|56312691|emb|CAI07336.1|(56312691); crotonase (Marinomonas sp. MED121) gi|86166463|gb|EAQ67729.1|(86166463); crotonase (Marinomonas sp. MED121) gi|87118829|ref|ZP_01074728.1|(87118829); crotonase (Roseovarius sp. 217) gi|85705898|ref|ZP_01036994.1|(85705898); crotonase (Roseovarius sp. 217) gi|85669486|gb|EAQ24351.1|(85669486); crotonase gi|1055218|gb|AAA95967.1|(1055218); 3-hydroxybutyryl-CoA dehydratase (Crotonase) gi|1706153|sp|P52046.1|CRT_CLOAB(1706153); Crotonase (3-hydroxybutyryl-COA dehydratase) (Clostridium acetobutylicum ATCC 824) gi|15025745|gb|AAK80658.1|AE007768_12 (15025745) each sequence associated with the accession number is incorporated herein by reference in its entirety. SEQ ID NO:22 sets forth an exemplary crt polypeptide sequence. In certain embodiments, the crotonase can have an amino acid sequence that is substantially identical to the amino acid sequence of SEQ ID NO:22 and having crotonase activity. For example, the disclosure includes polypeptides having at least about 80% identity, at least about 85%, at least about 90%, at least about 91%, at least about 92%, at least about 93%, at least about 94%, at least about 95%, at least about 96%, at least about 97%, at least about 98%, and at least about 99% identity to SEQ ID NO:22 and having crotonase. In other embodiments, the crotonase can have an amino acid sequence derived from the amino acid sequence of SEQ ID NO:22 by substitution, deletion, addition, or insertion of 1 or more amino acid(s) (e.g., 1-10) and having crotonase activity.

In another embodiment, an NADPH dependent enzyme can be used in the conversion of acetoacetyl-CoA to 3-hydroxybutyryl-CoA versus the NADH dependent enzyme of Hbd. Acetoacetyl-CoA reductase, PhB (e.g., R. eutropha phaB) (EC 1.1.1.36) generates 3-hydroxybutyryl-CoA from acetoacetyl-CoA and NADPH. In certain embodiments, the Acetoacetyl-CoA reductase can have an amino acid sequence that is substantially identical to the amino acid sequence of SEQ ID NO:30 and which has acetoacetyl-CoA reductase activity. For example, the disclosure includes polypeptides having at least about 80% identity, at least about 85%, at least about 90%, at least about 910, at least about 920, at least about 930, at least about 940, at least about 950, at least about 960, at least about 970, at least about 980, and at least about 99% identity to SEQ ID NO:30 and having Acetoacetyl-CoA reductase activity. In other embodiments, the acetoacetyl-CoA reductase can have an amino acid sequence derived from the amino acid sequence of SEQ ID NO:30 by substitution, deletion, addition, or insertion of 1 or more amino acid(s) (e.g., 1-10) and wherein the polypeptide has acetoacetyl-CoA reductase activity.

In a further embodiment, a polypeptide that converts (R)-3-hydroxybutyryl-CoA to crotonyl-CoA is used in combination with an acetoacetyl-coA reductase such as PhB. An example of an enzyme that can convert (R)-3-hydroxybutyryl-CoA to crotonyl-CoA is enoyl-CoA hydratase. A phaJ gene encodes an enzyme the converts (R)-3-hydroxybutyryl-CoA to crotonyl-CoA. In some embodiments, an enoyl-CoA hydratase gene is an Aeromonas caviae enoyl-CoA hydratase gene or a Pseudomonas aeruginosa enoyl-CoA hydratase gene. In some embodiments, the Pseudomonas aeruginosa enoyl-CoA hydratase gene is a Pseudomonas aeruginosa phaJ1 gene (gene PA3302) or a Pseudomonas aeruginosa phaJ2 gene (gene PA1018). A phaJ gene or homolog thereof can be derived from a number of microorganisms including, but not limited to, Aeromonas caviae. In certain embodiments, the enoyl-CoA hydratase can have an amino acid sequence that is substantially identical to the amino acid sequence of SEQ ID NO:28 and having enoyl-CoA hydratase activity. For example, the disclosure includes polypeptides having at least about 80% identity, at least about 85%, at least about 90%, at least about 91%, at least about 92%, at least about 93%, at least about 94%, at least about 95%, at least about 96%, at least about 97%, at least about 98%, and at least about 99% identity to SEQ ID NO:28 and having enoyl-CoA hydratase activity. In other embodiments, the enoyl-CoA hydratase can have an amino acid sequence derived from the amino acid sequence of SEQ ID NO:28 by substitution, deletion, addition, or insertion of 1 or more amino acid(s) (e.g., 1-10) and having enoyl-CoA hydratase activity.

In yet another embodiment, a recombinant microorganism provided herein includes expression or elevated expression of a crotonyl-CoA reductase as compared to a parental microorganism. The microorganism produces a metabolite that includes butyryl-CoA from a substrate that includes crotonyl-CoA. The crotonyl-CoA reductase can be encoded by a ccr gene, polynucleotide or homolog thereof. The ccr gene or polynucleotide can be derived from the genus Streptomyces.

Crotonyl-coA reductase catalyzes the reduction of crotonyl-CoA to butyryl-CoA. Depending upon the organism used a heterologous crotonyl-coA reductase can be engineered for expression in the organism. Alternatively, a native Crotonyl-coA reductase can be overexpressed. Crotonyl-coA reductase is encoded in S. coelicolor by ccr. CCR homologs and variants are known. For examples, such homologs and variants include, for example, crotonyl CoA reductase (Streptomyces coelicolor A3(2)) gi|21224777|ref|NP_630556.1|(21224777); crotonyl CoA reductase (Streptomyces coelicolor A3(2)) gi|4154068|emb|CAA22721.1|(4154068); crotonyl-CoA reductase (Methylobacterium sp. 4-46) gi|168192678|gb|ACA14625.1|(168192678); crotonyl-CoA reductase (Dinoroseobacter shibae DFL 12) gi|159045393|ref|YP_001534187.1|(159045393); crotonyl-CoA reductase (Salinispora arenicola CNS-205) gi|159039522|ref|YP_001538775.1|(159039522); crotonyl-CoA reductase (Methylobacterium extorquens PA1) gi|163849740|ref|YP_001637783.1|(163849740); crotonyl-CoA reductase (Methylobacterium extorquens PA1) gi|163661345|gb|ABY28712.1|(163661345); crotonyl-CoA reductase (Burkholderia ambifaria AMMD) gi|115360962|ref|YP_778099.1|(115360962); crotonyl-CoA reductase (Parvibaculum lavamentivorans DS-1) gi|154252073|ref|YP_001412897.1|(154252073); Crotonyl-CoA reductase (Silicibacter sp. TM1040) gi|99078082|ref|YP_611340.1|(99078082); crotonyl-CoA reductase (Xanthobacter autotrophicus Py2) gi|154245143|ref|YP_001416101.1|(154245143); crotonyl-CoA reductase (Nocardioides sp. JS614) gi|119716029|ref|YP_922994.1|(119716029): crotonyl-CoA reductase (Nocardioides sp. JS614) gi|119536690|gb|ABL81307.1|(119536690); crotonyl-CoA reductase (Salinispora arenicola CNS-205) gi|157918357|gb|ABV99784.1|(157918357); crotonyl-CoA reductase (Dinoroseobacter shibae DFL 12) gi|157913153|gb|ABV94586.1|(157913153); crotonyl-CoA reductase (Burkholderia ambifaria AMMD) gi|115286290|gb|ABI91765.1|(115286290); crotonyl-CoA reductase (Xanthobacter autotrophicus Py2) gi|154159228|gb|ABS66444.1|(154159228); crotonyl-CoA reductase (Parvibaculum lavamentivorans DS-1) gi|154156023|gb|ABS63240.1|(154156023); crotonyl-CoA reductase (Methylobacterium radiotolerans JCM 2831) gi|170654059|gb|ACB23114.1|(170654059); crotonyl-CoA reductase (Burkholderia graminis C4D1M) gi|170140183|gb|EDT08361.1|(170140183); crotonyl-CoA reductase (Methylobacterium sp. 4-46) gi|168198006|gb|ACA19953.1|(168198006); crotonyl-CoA reductase (Frankia sp. EANlpec) gi|158315836|ref|YP_001508344.1|(158315836), each sequence associated with the accession number is incorporated herein by reference in its entirety. For example, the disclosure provides the polypeptide sequences of a number of ccr polypeptides of the disclosure (e.g., see SEQ ID Nos: 4, 6, 8, 10, 12, 14, or 16). In addition, the disclosure includes modified ccr polypeptides and homologs thereof having at least 90%, 95%, 98%, or 99% identity to SEQ ID NO:4, 6, 8, 10, 12, 14, or 16 and having crotonyl-CoA reductase activity.

In another embodiment, the microorganism comprises a heterologous trans-2-enoyl-CoA reductase (ter) rather than or in addition to a ccr gene. Trans-2-enoyl-CoA reductase or TER is a protein that is capable of catalyzing the conversion of crotonyl-CoA to butyryl-CoA. In certain embodiments, the recombinant microorganism expresses a TER which catalyzes the same reaction as Bcd/EtfA/EtfB from Clostridia and other bacterial species. Mitochondrial TER from E. gracilis has been described, and many TER proteins and proteins with TER activity derived from a number of species have been identified forming a TER protein family (U.S. Pat. Publ. No. 2007/0022497 to Cirpus et al.; Hoffmeister et al., J. Biol. Chem., 280:4329-4338, 2005, both of which are incorporated herein by reference in their entirety). Trans-2-enoyl-CoA reductase is present in T. denticola, F. succinogens, T. vincentii or F. johnsoniae. In T. denticoloa TER has the accession number Q73Q47. In one embodiment the F. succinogens TER comprises the sequence set forth in SEQ ID NOs:23, 24, 25 (which can have a M11K mutation) or 26. In addition, TER proteins can also be identified by generally well known bioinformatics methods, such as BLAST. A truncated cDNA of the E. gracilis gene has been functionally expressed in E. coli. This cDNA or the genes of homologues from other microorganisms can be expressed together with the n-butanol pathway genes described herein to produce n-butanol in E. coli, S. cerevisiae or other recombinant microorganism as described herein.

TER proteins can also be identified by generally well known bioinformatics methods, such as BLAST. Examples of TER proteins include, but are not limited to, TERs from species such as: Euglena spp. including, but not limited to, E. gracilis, Aeromonas spp. including, but not limited, to A. hydrophila, Psychromonas spp. including, but not limited to, P. ingrahamii, Photobacterium spp. including, but not limited, to P. profundum, Vibrio spp. including, but not limited, to V angustum, V. cholerae, V alginolyticus, V parahaemolyticus, V vulnificus, V fischeri, V splendidus, Shewanella spp. including, but not limited to, S. amazonensis, S. woodyi, S. frigidimarina, S. paeleana, S. baltica, S. denitrificans, Oceanospirillum spp., Xanthomonas spp. including, but not limited to, X oryzae, X campestris, Chromohalobacter spp. including, but not limited, to C. salexigens, Idiomarina spp. including, but not limited, to I. baltica, Pseudoalteromonas spp. including, but not limited to, P. atlantica, Alteromonas spp., Saccharophagus spp. including, but not limited to, S. degradans, S. marine gamma proteobacterium, S. alpha proteobacterium, Pseudomonas spp. including, but not limited to, P. aeruginosa, P. putida, P. fluorescens, Burkholderia spp. including, but not limited to, B. phytofirmans, B. cenocepacia, B. cepacia, B. ambifaria, B. vietnamensis, B. multivorans, B. dolosa, Methylbacillus spp. including, but not limited to, M. flageliatus, Stenotrophomonas spp. including, but not limited to, S. maltophilia, Congregibacter spp. including, but not limited to, C. litoralis, Serratia spp. including, but not limited to, S. proteamaculans, Marinomonas spp., Xytella spp. including, but not limited to, X fastidiosa, Reinekea spp., Colweffia spp. including, but not limited to, C. psychrerythraea, Yersinia spp. including, but not limited to, Y. pestis, Y. pseudotuberculosis, Methylobacillus spp. including, but not limited to, M. flagellatus, Cytophaga spp. including, but not limited to, C. hutchinsonii, Flavobacterium spp. including, but not limited to, F. johnsoniae, Microscilla spp. including, but not limited to, M marina, Polaribacter spp. including, but not limited to, P. irgensii, Clostridium spp. including, but not limited to, C. acetobutylicum, C. beijerenckii, C. cellulolyticum, Coxiella spp. including, but not limited to, C. burnetii. In a further embodiment, the ter is derived from a Treponema denticola or F. succinogenes. In certain embodiments, the trans-2-enoyl-CoA reductase can have an amino acid sequence that is substantially identical to the amino acid sequence of SEQ ID NO: 23, 24, 25 or 26 and having trans-2-enoyl-CoA reductase activity. For example, the disclosure includes polypeptides having at least about 80% identity, at least about 85%, at least about 90%, at least about 91%, at least about 92%, at least about 93%, at least about 94%, at least about 95%, at least about 96%, at least about 97%, at least about 98%, and at least about 99% identity to SEQ ID NO:23, 24, 26, or 26 and having trans-2-enoyl-CoA reductase. In other embodiments, the trans-2-enoyl-CoA reductase can have an amino acid sequence derived from the amino acid sequence of SEQ ID NO:23, 24, 25, or 26 by substitution, deletion, addition, or insertion of 1 or more amino acid(s) (e.g., 1-10) and having trans-2-enoyl-CoA reductase activity.

In another embodiment, a recombinant microorganism of the disclosure can comprise recombinant coenzyme-A-acylating propionaldehyde dehydrogenase activity. The coenzyme-A-acylating propionaldehyde dehydrogenase (PduP) generates butyraldehyde from butyryl-CoA and NADPH. In certain embodiments, the coenzyme-A-acylating propionaldehyde dehydrogenase can have an amino acid sequence that is substantially identical to the amino acid sequence of SEQ ID NO: 34 and having coenzyme-A-acylating propionaldehyde dehydrogenase activity. For example, the disclosure includes polypeptides having at least about 80% identity, at least about 85%, at least about 90%, at least about 91%, at least about 92%, at least about 93%, at least about 94%, at least about 95%, at least about 96%, at least about 97%, at least about 98%, and at least about 99% identity to any one of SEQ ID NO:34, 45, 47, 49, 51 and 53 and having coenzyme-A-acylating propionaldehyde dehydrogenase activity. In other embodiments, the coenzyme-A-acylating propionaldehyde dehydrogenase can have an amino acid sequence derived from the amino acid sequence of SEQ ID NO:34, 44, 46, 48, 50 or 52 by substitution, deletion, addition, or insertion of 1 or more amino acid(s) (e.g., 1-10) and having coenzyme-A-acylating propionaldehyde dehydrogenase activity. Various homologs of SEQ ID NO:34 are known and include, for example, PduP_A.hyd (YP_855873; SEQ ID NO:43 and 44), PduP_K.pne (YP_001336844; SEQ ID NO:45 and 46), PduP_L.bre (YP_795711; SEQ ID NO:47 and 48), PduP_L.mon (NP_464690; SEQ ID NO:49 and 50), PduP_P.gin (YP_001928839; SEQ ID NO:51 and 52), PduP_S.ent (AAD39015; SEQ ID NO:33 and 34). Other homologs include the nucleic acid and polypeptide sequences set forth in SEQ ID NOs:53-80), which can be used in the methods and recombinant microorganisms of the disclosure.

E. coli contains a native gene (yqhD) that was identified as a 1,3-propanediol dehydrogenase (U.S. Pat. No. 6,514,733). The yqhD gene, given as SEQ ID NO:31, has 40% identity to the gene adhB in Clostridium, a probable NADH-dependent butanol dehydrogenase. In certain embodiments, the 1,3-propanediol dehydrogenase can have an amino acid sequence that is substantially identical to the amino acid sequence of SEQ ID NO: 32 and having 1,3-propanediol dehydrogenase activity. For example, the disclosure includes polypeptides having at least about 80% identity, at least about 85%, at least about 90%, at least about 91%, at least about 92%, at least about 93%, at least about 94%, at least about 95%, at least about 96%, at least about 97%, at least about 98%, and at least about 99% identity to SEQ ID NO:32 and having 1,3-propanediol dehydrogenase activity. In other embodiments, the 1,3-propanediol dehydrogenase can have an amino acid sequence derived from the amino acid sequence of SEQ ID NO:32 by substitution, deletion, addition, or insertion of 1 or more amino acid(s) (e.g., 1-10) and having 1,3-propanediol dehydrogenase activity.

In an alternate embodiment, rather than utilizing YghD, a recombinant microorganism provided herein can comprise expression or elevated expression of an alcohol dehydrogenase (ADHE2) as compared to a parental microorganism. The recombinant microorganism produces a metabolite that includes butanol from a substrate that includes butyryl-CoA. The alcohol dehydrogenase can be encoded by bdhA/bdhB polynucleotide or homolog thereof, an aad gene, polynucleotide or homolog thereof, or an adhE2 gene, polynucleotide or homolog thereof. The aad gene or adhE2 gene or polynucleotide can be derived from Clostridium acetobutylicum. Aldehyde/alcohol dehydrogenase catalyzes the conversion of butyryl-CoA to butyraldehyde and butyraldehyde to 1-butanol. In one embodiment, the aldehyde/alcohol dehydrogenase catalyzes the conversion of butyryl-CoA to butyraldehyde and butyraldehyde to 1-butanol. Depending upon the organism used a heterologous aldehyde/alcohol dehydrogenase can be engineered for expression in the organism. Alternatively, a native aldehyde/alcohol dehydrogenase can be overexpressed. aldehyde/alcohol dehydrogenase is encoded in C. acetobuylicum by adhE (e.g., an adhE2). ADHE (e.g., ADHE2) homologs and variants are known. For examples, such homologs and variants include, for example, aldehyde-alcohol dehydrogenase (Clostridium acetobutylicum) gi|3790107|gb|AAD04638.1|(3790107); aldehyde-alcohol dehydrogenase (Clostridium botulinum A str. ATCC 3502) gi|148378348|ref|YP_001252889.1|(148378348); Aldehyde-alcohol dehydrogenase (Includes: Alcohol dehydrogenase (ADH) Acetaldehyde dehydrogenase (acetylating) (ACDH) gi|19858620|sp|P33744.3|ADHE_CLOAB(19858620); Aldehyde dehydrogenase (NAD+) (Clostridium acetobutylicum ATCC 824) gi|15004865|ref|NP_149325.1|(15004865); alcohol dehydrogenase E (Clostridium acetobutylicum) gi|298083|emb|CAA51344.1|(298083); Aldehyde dehydrogenase (NAD+) (Clostridium acetobutylicum ATCC 824) gi|14994477|gb|AAK76907.1|AE001438_160(14994477); aldehyde/alcohol dehydrogenase (Clostridium acetobutylicum) gi|12958626|gb|AAK09379.1|AF321779_1(12958626); Aldehyde-alcohol dehydrogenase, ADHE1 (Clostridium acetobutylicum ATCC 824) gi|15004739|ref|NP_149199.1|(15004739); Aldehyde-alcohol dehydrogenase, ADHE1 (Clostridium acetobutylicum ATCC 824) gi|14994351|gb|AAK76781.1|AE001438_34(14994351); aldehyde-alcohol dehydrogenase E (Clostridium perfringens str. 13) gi|18311513|ref|NP_563447.1|(18311513); aldehyde-alcohol dehydrogenase E (Clostridium perfringens str. 13) gi|18146197|dbj|BAB82237.1|(18146197), each sequence associated with the accession number is incorporated herein by reference in its entirety.

In one embodiment, the recombinant microorganism expresses (i) an AccABCD or homolog thereof, (ii) an NphT7 or homolog thereof, (iii) a PhaB/PhaJ or homologs thereof, (iv) a Ter or homolog thereof, (v) a PduP or homolog thereof, and (vi) and a YghD or homolog thereof. In a further embodiment, the recombinant microorganism may include a reduction, lack of expression or a knockout of a gene selected from the group consisting of enzyme that catalyzes the NADH-dependent conversion of pyruvate to D-lactate (e.g., ldhA); (ii) an enzyme that promotes catalysis of fumarate and succinate interconversion (e.g., frdBC); (iii) an oxygen transcription regulator; (iv) an enzyme that catalyzes the conversion of acetyl-coA to acetyl-phosphate (e.g., pta) and (v) any combination of (i)-(iv).

In yet another embodiment, the microorganism comprises expression or over expression or one or more or all of the following AccABCD, npHT7, phaB, PhaJ, Ter or Ccr, PduP, and/or yqhD (or homologs of any of the foregoing). In yet other embodiments, the microorganism comprises one or more knockouts selected from the group consisting of (i) an enzyme that catalyzes the NADH-dependent conversion of pyruvate to D-lactate (e.g., ldhA); (ii) an enzyme that promotes catalysis of fumarate and succinate interconversion (e.g., frdBC); (iii) an oxygen transcription regulator; and (iv) an enzyme that catalyzes the conversion of acetyl-coA to acetyl-phosphate (e.g., pta) and/or an alcohol oxidoreductase (e.g., adhE).

In another embodiment, microorganisms are described that are capable of metabolizing a carbon source for producing a biofuel alcohol such as n-butanol at a yield of at least 4% of theoretical, and, in some cases, a yield of over 50% of theoretical. As used herein, the term “yield” refers to the molar yield. For example, the yield equals 100% when one mole of glucose is converted to one mole of n-butanol. In particular, the term “yield” is defined as the mole of product obtained per mole of carbon source monomer and may be expressed as percent. Unless otherwise noted, yield is expressed as a percentage of the theoretical yield. “Theoretical yield” is defined as the maximum moles of product that can be generated per a given mole of substrate as dictated by the stoichiometry of the metabolic pathway used to make the product. In one embodiment, the yield is at least 4%, 5%, 6%, 7%, 8%, 9%, 10%, 11%, 12 or more. In another example, the yield of a recombinant microorganism can be from 5% to 90%.

In another embodiment, the disclosure provides a culture of recombinant microorganisms of the disclosure comprising a population that is substantially homogenous (e.g., from about 70-100% homogenous). In another embodiment, a culture can comprise a combination of microorganism each having distinct biosynthetic pathways that produced metabolites that can be used by at least one other microorganism in culture leading to the production of n-butanol. In these embodiments, at least one “population” of microorganism comprises a coenzyme-A-acylating propionaldehyde dehydrogenase or homolog thereof that is an oxygen tolerant CoA-acylating aldehyde dehydrogenases.

The disclosure identifies genes useful in the methods, compositions and organisms of the disclosure; however it will be recognized that absolute identity to such genes is not necessary. For example, changes in a particular gene or polynucleotide comprising a sequence encoding a polypeptide or enzyme can be performed and screened for activity. Typically such changes comprise conservative mutation and silent mutations. Such modified or mutated polynucleotides and polypeptides can be screened for expression of a functional enzyme activity using methods known in the art.

Due to the inherent degeneracy of the genetic code, other polynucleotides which encode substantially the same or a functionally equivalent polypeptide can also be used to clone and express the polynucleotides encoding such enzymes.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, a process sometimes called “codon optimization” or “controlling for species codon bias.”

Optimized coding sequences containing codons preferred by a particular prokaryotic or eukaryotic host (see also, Murray et al. (1989) Nucl. Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al. (1996) Nucl. Acids Res. 24: 216-218). Methodology for optimizing a nucleotide sequence for expression in a plant is provided, for example, in U.S. Pat. No. 6,015,891, and the references cited therein.

Those of skill in the art will recognize that, due to the degenerate nature of the genetic code, a variety of DNA compounds differing in their nucleotide sequences can be used to encode a given enzyme of the disclosure. The native DNA sequence encoding the biosynthetic enzymes described above are referenced herein merely to illustrate an embodiment of the disclosure, and the disclosure includes DNA compounds of any sequence that encode the amino acid sequences of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as they modified or variant polypeptides have the enzymatic anabolic or catabolic activity of the reference polypeptide. Furthermore, the amino acid sequences encoded by the DNA sequences shown herein merely illustrate embodiments of the disclosure.

In addition, homologs of enzymes useful for generating metabolites are encompassed by the microorganisms and methods provided herein. The term “homologs” used with respect to an original enzyme or gene of a first family or species refers to distinct enzymes or genes of a second family or species which are determined by functional, structural or genomic analyses to be an enzyme or gene of the second family or species which corresponds to the original enzyme or gene of the first family or species. Most often, homologs will have functional, structural or genomic similarities. Techniques are known by which homologs of an enzyme or gene can readily be cloned using genetic probes and PCR. Identity of cloned sequences as homolog can be confirmed using functional assays and/or by genomic mapping of the genes. A large number of homologs of the enzymes described herein can be readily identified by one of skill in the art using available electronic databases through the World-Wide-Web. Such homologs can be easily identified based upon (i) the sequences provided herein, (ii) the enzyme names provided herein and (iii) the reactions described herein.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences).

As used herein, two proteins (or a region of the proteins) are substantially homologous when the amino acid sequences have at least about 30%, 40%, 50% 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (see, e.g., Pearson et al., 1994, hereby incorporated herein by reference).

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which can also be referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1.

A typical algorithm used comparing a molecule sequence to a database containing a large number of sequences from different organisms is the computer program BLAST (Altschul, 1990; Gish, 1993; Madden, 1996; Altschul, 1997; Zhang, 1997), especially blastp or tblastn (Altschul, 1997). Typical parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

When searching a database containing sequences from a large number of different organisms, it is typical to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences (Pearson, 1990, hereby incorporated herein by reference). For example, percent sequence identity between amino acid sequences can be determined using FASTA with its default parameters (a word size of 2 and the PAM250 scoring matrix), as provided in GCG Version 6.1, hereby incorporated herein by reference.

It is understood that a range of microorganisms can be modified to include a recombinant metabolic pathway suitable for the production of biofuel alcohols and chemicals e.g., butyraldehyde, butanol and metabolites thereof. It is also understood that various microorganisms can act as “sources” for genetic material encoding target enzymes suitable for use in a recombinant microorganism provided herein. The term “microorganism” includes prokaryotic and eukaryotic photosynthetic microbial species. The terms “microbial cells” and “microbes” are used interchangeably with the term microorganism.

“Bacteria”, or “eubacteria”, refers to a domain of prokaryotic organisms. Bacteria include at least 11 distinct groups as follows: (1) Gram-positive (gram⁺) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

“Gram-negative bacteria” include cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium.

“Gram positive bacteria” include cocci, nonsporulating rods, and sporulating rods. The genera of gram positive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

Photoautotrophic bacteria are typically Gram-negative rods which obtain their energy from sunlight through the processes of photosynthesis. In this process, sunlight energy is used in the synthesis of carbohydrates, which in recombinant photoautotrophs can be further used as intermediates in the synthesis of biofuels. In other embodiment, the photoautotrophs serve as a source of carbohydrates for use by non-photosynthetic microorganism (e.g., recombinant E. coli) to produce biofuels by a metabolically engineered microorganism. Certain photoautotrophs called anoxygenic photoautotrophs grow only under anaerobic conditions and neither use water as a source of hydrogen nor produce oxygen from photosynthesis. Other photoautotrophic bacteria are oxygenic photoautotrophs. These bacteria are typically cyanobacteria. They use chlorophyll pigments and photosynthesis in photosynthetic processes resembling those in algae and complex plants. During the process, they use water as a source of hydrogen and produce oxygen as a product of photosynthesis.

Cyanobacteria include various types of bacterial rods and cocci, as well as certain filamentous forms. The cells contain thylakoids, which are cytoplasmic, platelike membranes containing chlorophyll. The organisms produce heterocysts, which are specialized cells believed to function in the fixation of nitrogen compounds.

The term “recombinant microorganism” and “recombinant host cell” are used interchangeably herein and refer to microorganisms that have been genetically modified to express or over-express endogenous nucleic acid sequences, or to express non-endogenous sequences, such as those included in a vector. The nucleic acid sequence generally encodes a target enzyme involved in a metabolic pathway for producing a desired metabolite as described above. Accordingly, recombinant microorganisms described herein have been genetically engineered to express or over-express target enzymes not previously expressed or over-expressed by a parental microorganism. It is understood that the terms “recombinant microorganism” and “recombinant host cell” refer not only to the particular recombinant microorganism but to the progeny or potential progeny of such a microorganism.

A “parental microorganism” refers to a cell used to generate a recombinant microorganism. The term “parental microorganism” describes a cell that occurs in nature, i.e. a “wild-type” cell that has not been genetically modified. The term “parental microorganism” also describes a cell that has been genetically modified but which does not express or over-express a target enzyme e.g., an enzyme involved in the biosynthetic pathway for the production of a desired metabolite such as 1-propanol, isobutanol, 1-butanol, 2-methyl 1-butanol, 3-methyl 1-butanol or 2-phenylethanol. For example, a wild-type microorganism can be genetically modified to express or over express a first target enzyme such as thiolase. This microorganism can act as a parental microorganism in the generation of a microorganism modified to express or over-express a second target enzyme e.g., hydroxybutyryl CoA dehydrogenase. In turn, the microorganism modified to express or over express e.g., thiolase and hydroxybutyryl CoA dehydrogenase can be modified to express or over express a third target enzyme e.g., crotonase. Accordingly, a parental microorganism functions as a reference cell for successive genetic modification events. Each modification event can be accomplished by introducing a nucleic acid molecule in to the reference cell. The introduction facilitates the expression or over-expression of a target enzyme. It is understood that the term “facilitates” encompasses the activation of endogenous nucleic acid sequences encoding a target enzyme through genetic modification of e.g., a promoter sequence in a parental microorganism. It is further understood that the term “facilitates” encompasses the introduction of exogenous nucleic acid sequences encoding a target enzyme in to a parental microorganism.

In another embodiment a method of producing a recombinant microorganism that converts a suitable carbon substrate to e.g., n-butanol or a metabolite downstream or upstream in the metabolic pathway is provided. The method includes transforming a microorganism with one or more recombinant nucleic acid sequences as described above and elsewhere herein. Nucleic acid sequences that encode enzymes useful for generating metabolites including homologs, variants, fragments, related fusion proteins, or functional equivalents thereof, are used in recombinant nucleic acid molecules that direct the expression of such polypeptides in appropriate host cells, such as bacterial or yeast cells. It is understood that the addition of sequences which do not alter the encoded activity of a nucleic acid molecule, such as the addition of a non-functional or non-coding sequence, is a conservative variation of the basic nucleic acid. The “activity” of an enzyme is a measure of its ability to catalyze a reaction resulting in a metabolite, i.e., to “function”, and may be expressed as the rate at which the metabolite of the reaction is produced. For example, enzyme activity can be represented as the amount of metabolite produced per unit of time or per unit of enzyme (e.g., concentration or weight), or in terms of affinity or dissociation constants.

A “protein” or “polypeptide”, which terms are used interchangeably herein, comprises one or more chains of chemical building blocks called amino acids that are linked together by chemical bonds called peptide bonds. An “enzyme” means any substance, composed wholly or largely of protein, that catalyzes or promotes, more or less specifically, one or more chemical or biochemical reactions. The term “enzyme” can also refer to a catalytic polynucleotide (e.g., RNA or DNA). A “native” or “wild-type” protein, enzyme, polynucleotide, gene, or cell, means a protein, enzyme, polynucleotide, gene, or cell that occurs in nature.

It is understood that the nucleic acid sequences described above include “genes” and that the nucleic acid molecules described above include “vectors” or “plasmids.” For example, a nucleic acid sequence encoding a keto thiolase can be encoded by an atoB gene or homolog thereof, or an fadA gene or homolog thereof. Accordingly, the term “gene”, also called a “structural gene” refers to a nucleic acid sequence that codes for a particular sequence of amino acids, which comprise all or part of one or more proteins or enzymes, and may include regulatory (non-transcribed) DNA sequences, such as promoter sequences, which determine for example the conditions under which the gene is expressed. The transcribed region of the gene may include untranslated regions, including introns, 5′-untranslated region (UTR), and 3′-UTR, as well as the coding sequence. The term “nucleic acid” or “recombinant nucleic acid” refers to polynucleotides such as deoxyribonucleic acid (DNA), and, where appropriate, ribonucleic acid (RNA). The term “expression” with respect to a gene sequence refers to transcription of the gene and, as appropriate, translation of the resulting mRNA transcript to a protein. Thus, as will be clear from the context, expression of a protein results from transcription and translation of the open reading frame sequence.

The term “operon” refers two or more genes which are transcribed as a single transcriptional unit from a common promoter. In some embodiments, the genes comprising the operon are contiguous genes. It is understood that transcription of an entire operon can be modified (i.e., increased, decreased, or eliminated) by modifying the common promoter. Alternatively, any gene or combination of genes in an operon can be modified to alter the function or activity of the encoded polypeptide. The modification can result in an increase in the activity of the encoded polypeptide. Further, the modification can impart new activities on the encoded polypeptide. Exemplary new activities include the use of alternative substrates and/or the ability to function in alternative environmental conditions.

“Transformation” refers to the process by which a vector is introduced into a host cell. Transformation (or transduction, or transfection), can be achieved by any one of a number of means including electroporation, microinjection, biolistics (or particle bombardment-mediated delivery), or agrobacterium mediated transformation.

A “vector” is any means by which a nucleic acid can be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes,” that is, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not episomal in nature, or it can be an organism which comprises one or more of the above polynucleotide constructs such as an agrobacterium or a bacterium.

The disclosure provides nucleic acid molecules in the form of recombinant DNA expression vectors or plasmids, as described in more detail below, that encode one or more target enzymes. Generally, such vectors can either replicate in the cytoplasm of the host microorganism or integrate into the chromosomal DNA of the host microorganism. In either case, the vector can be a stable vector (i.e., the vector remains present over many cell divisions, even if only with selective pressure) or a transient vector (i.e., the vector is gradually lost by host microorganisms with increasing numbers of cell divisions). The disclosure provides DNA molecules in isolated (i.e., not pure, but existing in a preparation in an abundance and/or concentration not found in nature) and purified (i.e., substantially free of contaminating materials or substantially free of materials with which the corresponding DNA would be found in nature) forms.

Provided herein are methods for the heterologous expression of one or more of the biosynthetic genes involved in biofuel alcohol and chemical production, e.g., n-butanol biosynthesis or in the biosynthesis of a metabolite in the metabolic pathway to produce n-butanol and recombinant DNA expression vectors useful in the method. Thus, included within the scope of the disclosure are recombinant expression vectors that include polynucleotides encoding polypeptides having an enzymatic activity in a desired metabolic pathway. The term expression vector refers to a nucleic acid that can be introduced into a host microorganism or cell-free transcription and translation system. An expression vector can be maintained permanently or transiently in a microorganism, whether as part of the chromosomal or other DNA in the microorganism or in any cellular compartment, such as a replicating vector in the cytoplasm. An expression vector also comprises a promoter that drives expression of an RNA, which typically is translated into a polypeptide in the microorganism or cell extract. For efficient translation of RNA into protein, the expression vector also typically contains a ribosome-binding site sequence positioned upstream of the start codon of the coding sequence of the gene to be expressed. Other elements, such as enhancers, secretion signal sequences, transcription termination sequences, and one or more marker genes by which host microorganisms containing the vector can be identified and/or selected, may also be present in an expression vector. Selectable markers, i.e., genes that confer antibiotic resistance or sensitivity, are used and confer a selectable phenotype on transformed cells when the cells are grown in an appropriate selective medium.

The various components of an expression vector can vary widely, depending on the intended use of the vector and the host cell(s) in which the vector is intended to replicate or drive expression. Expression vector components suitable for the expression of genes and maintenance of vectors in E. coli, yeast, Streptomyces, and other commonly used cells are widely known and commercially available. For example, suitable promoters for inclusion in the expression vectors of the disclosure include those that function in eukaryotic or prokaryotic host microorganisms. Promoters can comprise regulatory sequences that allow for regulation of expression relative to the growth of the host microorganism or that cause the expression of a gene to be turned on or off in response to a chemical or physical stimulus. For E. coli and certain other bacterial host cells, promoters derived from genes for biosynthetic enzymes, antibiotic-resistance conferring enzymes, and phage proteins can be used and include, for example, the galactose, lactose (lac), maltose, tryptophan (trp), beta-lactamase (bla), bacteriophage lambda PL, and T5 promoters. In addition, synthetic promoters, such as the tac promoter (U.S. Pat. No. 4,551,433), can also be used. For E. coli expression vectors, it is useful to include an E. coli origin of replication, such as from pUC, plP, pl, and pBR.

Thus, recombinant expression vectors contain at least one expression system, which, in turn, is composed of at least a portion of a biosynthetic gene coding sequences operably linked to a promoter and optionally termination sequences that operate to effect expression of the coding sequence in compatible host cells. The host cells are modified by transformation with the recombinant DNA expression vectors of the disclosure to contain the expression system sequences either as extrachromosomal elements or integrated into the chromosome.

Due to the inherent degeneracy of the genetic code, other nucleic acid sequences which encode substantially the same or a functionally equivalent amino acid sequence can also be used to clone and express the polynucleotides encoding such enzymes. As previously noted, the term “host cell” is used interchangeably with the term “recombinant microorganism” and includes any cell type which is suitable for producing e.g., n-butanol or a metabolite in a metabolic pathway to produce n-butanol and susceptible to transformation with a nucleic acid construct such as a vector or plasmid.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, a process sometimes called “codon optimization” or “controlling for species codon bias.”

A nucleic acid of the disclosure can be amplified using cDNA, mRNA or alternatively, genomic DNA, as a template and appropriate oligonucleotide primers according to standard PCR amplification techniques and those procedures described in the Examples section below. The nucleic acid so amplified can be cloned into an appropriate vector and characterized by DNA sequence analysis. Furthermore, oligonucleotides corresponding to nucleotide sequences can be prepared by standard synthetic techniques, e.g., using an automated DNA synthesizer.

It is also understood that an isolated nucleic acid molecule encoding a polypeptide homologous to the enzymes described herein can be created by introducing one or more nucleotide substitutions, additions or deletions into the nucleotide sequence encoding the particular polypeptide, such that one or more amino acid substitutions, additions or deletions are introduced into the encoded protein. Mutations can be introduced into the nucleic acid sequence by standard techniques, such as site-directed mutagenesis and PCR-mediated mutagenesis. In contrast to those positions where it may be desirable to make a non-conservative amino acid substitutions (see above), in some positions it is preferable to make conservative amino acid substitutions. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

In another embodiment a method for producing e.g., 1-propanol, isobutanol, 1-butanol, 2-methyl 1-butanol, 3-methyl 1-butanol or 2-phenylethanol is provided. The method includes culturing a recombinant photoautotroph or photoheterotroph microorganism(s) or culture comprising a photoautotroph or photoheterotroph and a recombinant non-photosynthetic or photoheterotroph microorganism as provided herein in the presence of a suitable substrate (e.g., CO₂) and under conditions suitable for the conversion of the substrate to 1-propanol, isobutanol, 1-butanol, 2-methyl 1-butanol, 3-methyl 1-butanol or 2-phenylethanol. The alcohol produced by a microorganism or culture provided herein can be detected by any method known to the skilled artisan. Culture conditions suitable for the growth and maintenance of a recombinant microorganism provided herein are described in the Examples below. The skilled artisan will recognize that such conditions can be modified to accommodate the requirements of each microorganism.

As previously discussed, general texts which describe molecular biological techniques useful herein, including the use of vectors, promoters and many other relevant topics, include Berger and Kimmel, Guide to Molecular Cloning Techniques, Methods in Enzymology Volume 152, (Academic Press, Inc., San Diego, Calif.) (“Berger”); Sambrook et al., Molecular Cloning—A Laboratory Manual, 2d ed., Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”). Examples of protocols sufficient to direct persons of skill through in vitro amplification methods, including the polymerase chain reaction (PCR), the ligase chain reaction (LCR), Q□-replicase amplification and other RNA polymerase mediated techniques (e.g., NASBA), e.g., for the production of the homologous nucleic acids of the disclosure are found in Berger, Sambrook, and Ausubel, as well as in Mullis et al. (1987) U.S. Pat. No. 4,683,202; Innis et al., eds. (1990) PCR Protocols: A Guide to Methods and Applications (Academic Press Inc. San Diego, Calif.) (“Innis”); Arnheim & Levinson (Oct. 1, 1990) C&EN 36-47; The Journal Of NIH Research (1991) 3: 81-94; Kwoh et al. (1989) Proc. Natl. Acad. Sci. USA 86: 1173; Guatelli et al. (1990) Proc. Nat'l. Acad. Sci. USA 87: 1874; Lomell et al. (1989) J. Clin. Chem 35: 1826; Landegren et al. (1988) Science 241: 1077-1080; Van Brunt (1990) Biotechnology 8: 291-294; Wu and Wallace (1989) Gene 4:560; Barringer et al. (1990) Gene 89:117; and Sooknanan and Malek (1995) Biotechnology 13: 563-564. Improved methods for cloning in vitro amplified nucleic acids are described in Wallace et al., U.S. Pat. No. 5,426,039. Improved methods for amplifying large nucleic acids by PCR are summarized in Cheng et al. (1994) Nature 369: 684-685 and the references cited therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase. See, e.g., Ausubel, Sambrook and Berger, all supra.

### Examples

### Materials and Methods

Chemicals and Reagents.

All chemicals were purchased either from Sigma-Aldrich (St. Louis, Mo.) or Fisher Scientifics (Pittsburgh, Pa.) unless otherwise specified. iProof high-fidelity DNA polymerase was purchased from Bio-Rad (Hercules, Calif.). Restriction enzymes, Phusion DNA polymerase, and ligases were purchased from New England Biolabs (Ipswich, Mass.). T5-Exonuclease was purchased from Epicentre Biotechnologies (Madison, Wis.). KOD and KOD xtreme DNA polymerases were purchased from EMD biosciences (Gibbstown, N.J.).

DNA Manipulations.

All chromosomal manipulations were carried out by homologous recombination of plasmid DNA into S. elongatus PCC 7942 genome at neutral site I (NSI) and II (NSII). All plasmids were constructed using Gibson isothermal DNA assembly method. Plasmids were constructed in E. coli XL-1 blue for propagation and storage (Table 1). Accession number for each enzyme used in this study is listed: NphT7 (BAJ10048), PhaB (AEI76813), PhaJ (032472), Ter (Q73Q47), YqhD (AAC76047), PduP_A.hyd (YP_855873), PduP_K.pne (YP_001336844), PduP_L.bre (YP_795711), PduP_L.mon (NP_464690), PduP_P.gin (YP_001928839), PduP_S.ent (AAD39015).

TABLE 1
Plasmids used in this disclosure

| Plasmid | Genotypes | Reference |
|---|---|---|
| pCDFDuet | Spec^{R}; CDF ori; P_{T7}::MCS | Novagen |
| pEL54 | Amp^{R}; ColE1 ori; P_{LlacO1}::atoB,bldh,yqhD,crt,hbd | Lan & Liao, 2012 |
| pIM8 | Kan^{R}; ColA ori; P_{LlacO1}::ter | Shen et al., 2011 |
| pCDF-pduP_ahyd | Spec^{R}; CDF ori; P_{T7}::pduP_A.hyd (his tagged) | This work |
| pCDF-pduP_kpne | Spec^{R}; CDF ori; P_{T7}::pduP_K.pne (his tagged) | This work |
| pCDF-pduP_lbre | Spec^{R}; CDF ori; P_{T7}::pduP_L.bre (his tagged) | This work |
| pCDF-pduP_lmon | Spec^{R}; CDF ori; P_{T7}::pduP_L.mon (his tagged) | This work |
| pCDF-pduP_sent | Spec^{R}; CDF ori; P_{T7}::pduP_P.gin (his tagged) | This work |
| pCDF-pduP_pgin | Spec^{R}; CDF ori; P_{T7}::pduP_S.ent (his tagged) | This work |
| pEL175 | Amp^{R}; ColE1 ori; P_{LlacO1}::atoB,ter,crt,hbd | This work |
| pEL178 | Kan^{R}; P15A ori; P_{LlacO1}::bldh,yqhD | This work |
| pEL179 | Kan^{R}; P15A ori; P_{LlacO1}::pduP_A.hyd,yqhD | This work |
| pEL180 | Kan^{R}; P15A ori; P_{LlacO1}::pduP_K.pne,yqhD | This work |
| pEL181 | Kan^{R}; P15A ori; P_{LlacO1}::pduP_L.bre,yqhD | This work |
| pEL182 | Kan^{R}; P15A ori; P_{LlacO1}::pduP_L.mon,yqhD | This work |
| pEL183 | Kan^{R}; P15A ori; P_{LlacO1}::pduP_P.gin,yqhD | This work |
| pEL184 | Kan^{R}; P15A ori; P_{LlacO1}::pduP_S.ent,yqhD | This work |
| pSR2 | Kan^{R}; NSII targeting; P_{LlacO1}::nphT7,pduP_L.bre,crt,hbd | This work |
| pSR3 | Kan^{R}; NSII targeting; P_{LlacO1}::nphT7,pduP_S.ent,crt,hbd | This work |
| pSR5 | Kan^{R}; NSII targeting; P_{LlacO1}::nphT7,pduP_L.mon,crt,hbd | This work |
| pSR6 | Spec^{R}; NSI targeting; P_{trc}::pduP_K.pne,yqhD | This work |
| pSR7 | Spec^{R}; NSI targeting; P_{trc}::pduP_L.bre,yqhD | This work |
| pSR8 | Spec^{R}; NSI targeting; P_{trc}::pduP_S.ent,yqhD | This work |
| pSR9 | Spec^{R}; NSI targeting; P_{trc}::pduP_L.mon,yqhD | This work |

Kan^{R}, kanamycin resistance; Amp^{R}, ampicillin resistance; Spec^{R}, spectinomycin resistance atoB (E. coli), thiolase; nphT7 (Streptomyces sp. strain CL190), acetoacetyl-CoA synthase; phaB (R. Eutropha), acetoacetyl-CoA reductase; phaJ (A. caviae), (R)-specific enoyl-CoA hydratase; hbd (C. acetobutylicum), 3-hydroxybutyryl-CoA dehydrogenase; crt (C. acetobutylicum), crotonase; ter (T. denticola), Trans-2-enoyl-CoA reductase; bldh (C. saccharoperbutylacetonicum), butyraldehyde dehydrogenase; yqhD (E. coli), NADP-dependent alcohol dehydrogenase; pduP (A. hydrophilia, K. pneumonia, L. brevis, L. monocytigenes, P. gingivalis, or S. enterica), CoA-acylating aldehyde dehydrogenase.

Culture Medium and Condition.

All S. elongatus PCC 7942 strains were grown in modified BG-11 (1.5 g/L NaNO₃, 0.0272 g/L CaCl₂.2H₂O, 0.012 g/L ferric ammonium citrate, 0.001 g/L Na₂EDTA, 0.040 g/L K₂HPO₄, 0.0361 g/L MgSO₄.7H₂O, 0.020 g/L Na₂CO₃, 1000× trace mineral (1.43 g H₃BO₃, 0.905 g/L MnCl₂.4H₂O, 0.111 g/L ZnSO₄.7H₂O, 0.195 g/L Na₂MoO₄.2H₂O, 0.0395 g CuSO₄.5H₂O, 0.0245 g Co(NO₃)₂.6H₂O), 0.00882 g/L sodium citrate dihydrate) agar (1.5% w/v) plates. All S. elongatus PCC 7942 strains were cultured in BG-11 medium containing 50 mM NaHCO₃ in 250 mL screw cap flasks. Cultures were grown under 48±2 pE/s/m² light measured by Licor quantum sensor (LI-250A equipped with LI-190 Quantum Sensor), supplied by 3 Lumichrome F30W-1XX 6500K 98CRI light tubes, at 29±1° C. Cell growth was monitored by measuring OD₇₃₀ with Beckman Coulter DU800 spectrophotometer.

Strain Construction and Transformation.

S. elongatus PCC 7942 strains were transformed by incubating cells at mid-log phase (OD₇₃₀ of 0.4 to 0.6) with 2 μg of plasmid DNA overnight in the dark. The culture was then spread on BG-11 plates supplemented with appropriate antibiotics for selection of successful recombination. For selection and culture maintenance, 20 μg/ml spectinomycin and 10 μg/ml kanamycin were added into BG-11 agar plates and BG-11 medium where appropriate. Colony PCR was used to verify integration of inserted genes into the recombinant strain. In all cases, two or three individual colonies were analyzed and propagated for downstream tests. Genotypes of recombinant S. elongatus are listed in Table 3. S. elongatus strain ETOH-KP, ETOH-LB, ETOH-LM, and ETOH-SE were constructed by transforming strain PCC 7942 with plasmids pSR6, pSR7, pSR9, and pSR8, respectively. Strains BUOH-LB, BUOH-SE, and BUOH-LM were constructed by transforming strain EL9 with plasmids pSR2, pSR3, and pSR5, respectively.

E. coli Anaerobic Growth Rescue.

Cells of E. coli strain JCL166 and its derivatives were cultured overnight in LB with appropriate antibiotics at 37° C. Overnight cultures (3 μL) were used to inoculate 3 mL of fresh LB media supplemented with 1% glucose and appropriate antibiotics (100 μg/mL ampicillin and 50 μg/mL kanamycin) and 0.1 mM IPTG in 10 mL BD vacutainers. A needle with Millipore PES filter was used to pierce through the rubber cap of the culture. The headspace of these cultures was then purged several times with 95% N₂/5% H₂ gas using an anaerobic chamber (Coy Lab Products, Grass Lake, Mich.). The needle with filter was removed in anaerobic chamber. These anaerobic cultures were then taken out of anaerobic chamber and incubated in 37° C. rotary shaker. Culture was analyzed 24 hours later for cell density and alcohol content measurement.

CoA-Acylating Aldehyde Dehydrogenase (PduP) Assay.

Protein purification was done by using His-Spin Protein miniprep purification kit from Zymo following manufacturer's manual. Overnight culture of E. coli strain BL-21(DE3) harboring different pCDF-pdup plasmid was used to inoculate fresh 20 ml LB. The newly inoculated culture was incubated at 37° C. until OD_{600 nm} reaches 0.6 which was then induced with 1 mM IPTG. The induced cultures were then incubated overnight in 30° C. rotary shaker to allow protein expression. The cultures were then harvested by centrifugation at 4,300×g for 20 min. The pellet was then resuspended with 1 mL of Zymo His-binding buffer and mixed with 1 mL of 0.1 mm glass beads (Biospec). The sample was then homogenated using TissueLyser II (Quiagen).

Enzyme assays were conducted by using Bio-Tek PowerWave XS microplate spectrophotometer. CoA-acylating aldehyde dehydrogenase activity was measured by the decrease of absorbance at 340 nm, corresponding to depletion of NADH. For determination of k_{cat} and Kₘ of the different aldehyde dehydrogenases, acetyl-CoA and butyryl-CoA concentrations were varied between 0.03 mM to 2 mM. The reaction mixture contained 50 mM potassium phosphate buffer at pH 7.15, 1 mM Dithiothreitol (DTT), 500 μM NADH, acetyl-CoA or butyryl-CoA at varying concentration, and enzyme. For determining specific activity for other chain length acyl-CoA, 500 μM was used. The mixture excluding acyl-CoA was incubated for 5 minutes at 30° C. The addition of the acyl-CoA initiated the reaction. k_{cat} and Kₘ were determined by nonlinear regression fitting to Michaelis-Menten equation using graphing software Origin.

Production of Ethanol and n-Butanol in Recombinant S. Elongates.

A loopful of S. elongatus PCC 7942 was used to inoculate fresh 50 mL BG-11. Initial cell density of culture was typically OD₇₃₀ 0.03 to 0.05. 500 mM IPTG was used to induce the growing culture at cell density OD_{730 nm} of 0.4 to 0.6 with 1 mM IPTG as final concentration.

For n-butanol production, 1 mL of culture was removed from the culture daily for cell density measurement and quantification of n-butanol. Every two days, additional 3 mL of culture was removed from the flask, and 5 mL of fresh BG-11 with 500 mM NaHCO₃, appropriate antibiotics, and IPTG were added back to the culture. This procedure ensures the carbon supply for S. elongatus.

For ethanol production, 5 mL of culture was removed every two days from the flask for cell density measurement and quantification of ethanol. 5 mL of fresh BG-11 with 500 mM NaHCO₃, appropriate antibiotics, and IPTG were added back to the culture to supply nutrients necessary.

Alcohol Quantification.

Culture samples (1 mL) were centrifuged for 5 minutes at 15,000×g. The supernatant (900 μL) was then mixed with 0.1% v/v 2-methyl-pentanol (100 μL) as internal standard. The mixture was then vortexed and directly analyzed on an Agilent GC 6850 system equipped with flame ionization detector and DB-FFAP capillary column (30 m, 0.32 mm i.d., 0.25 film thickness) from Agilent Technologies (Santa Clara, Calif.). Ethanol or n-butanol in the sample was identified and quantified by comparing to 0.1% v/v standard. The GC result was analyzed by Agilent software Chem Station (Rev.B.04.01 SP1). Amount of alcohol in the sample was then calculated based on the ratio of its integrated area and that of the 0.1% v/v standard.

Helium was used as the carrier gas with 9.52 psi inlet pressure. The injector and detector temperatures were maintained at 225° C. Injection volume was 1 μL. For ethanol quantification, the GC over was initially held at 60° C. for 2 minutes, and raised to 85° C. with temperature ramp of 50° C./min and kept at 85° C. for 2 minutes after which temperature was then raised to 235° C. with ramp rate of 45° C./min and kept for 3 minutes. For n-butanol quantification, the GC oven temperature was initially held at 85° C. for 3 minutes and then raised to 235° C. with a temperature ramp of 45° C./min. The GC oven was then maintained at 235° C. for 1 minute before completion of analysis. Column flow rate was 1.7 ml/min. DMF and water were used as organic and aqueous wash solvent, respectively.

Cumulative alcohol concentration is defined as the sum of daily productivities of the production period. Note that culture volume remained constant throughout production. 10% of alcohol was removed every two days due to nutrients replenishment. For example, feeding was done on day 2, therefore to calculate daily productivity of day 3, the difference between concentration of day 3 and 90% of day 2 were taken as the daily productivity for day 3.

n-Butanol Toxicity Test.

S. elongatus PCC7942 cells were grown according to the procedure in “culture medium and condition”. Once growth reached OD₇₃₀ about 2, the culture was diluted to OD₇₃₀ of 0.1 with 50 mL fresh BG11 medium containing 50 mM NaHCO₃ and appropriate antibiotics. Then, various amounts of n-butanol were added into the culture to achieve the desired concentration (0, 250, 500, 750, and 1000 mg/L). Growth was measured daily. Every two days, 5 mL of culture was removed and 5 mL fresh BG-11 containing 500 mM NaHCO₃, appropriate amounts of antibiotics and n-butanol was added back to the culture to ensure sufficient carbon supply.

CoA-Acylating Aldehyde Dehydrogenase PduP Catalyzes Butyryl-CoA Reduction.

To circumvent Bldh oxygen sensitivity in cyanobacteria, alternative CoA-acylating aldehyde dehydrogenases were identified. PduP, an enzyme identified from Salmonella enterica, is responsible for catalyzing the oxidation of propionaldehyde to propionyl-CoA, an important detoxification step for 1,2-propanediol degradation pathway. This enzyme is expressed in S. enterica under aerobic condition and has been previously demonstrated with in vitro activity for the oxidation of propionaldehyde to propionyl-CoA. In addition to S. enterica PduP, five more PduP homologues were cloned from Aeromonas hydrophila, Klebsiella pneumoniae, Lactobacillus brevis, Listeria monocytigenes, and Porphyromonas gingivalis identified by homology search with BLAST using PduP protein sequence. It was unclear whether or not PduP is efficient at catalyzing the reduction of acyl-CoA to the corresponding aldehyde, the reverse of its natural direction. It was also uncertain whether or not PduP acts on butyl-CoA, the four carbon substrate.

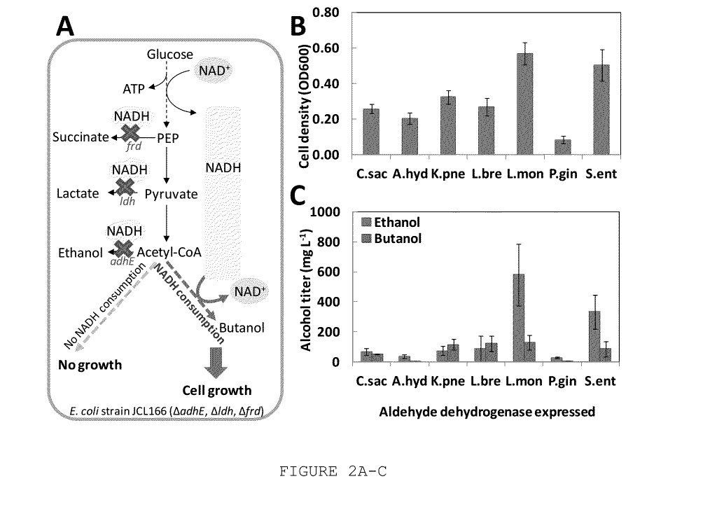

These PduP homologues were tested for n-butanol production utilizing an anaerobic growth rescue assay. E. coli strain JCL 166 (ΔadhE, ΔldhA, ΔfrdB) contains gene knock-outs for mixed acid fermentation. Fermentation pathways are essential for E. coli to recycle NADH back into to NAD⁺ for the Embden-Meyerhof-Parnas pathway to continue (FIG. 2A). As a result of these knock-outs, strain JCL166 cannot grow anaerobically unless complemented by an exogenous fermentation pathway such as n-butanol biosynthesis. The PduP homologues were expressed together with the rest of the Ter dependent n-butanol biosynthetic genes using plasmids pEL179, pEL180, pEL181, pEL182, pEL183, and pEL184 for expressing each pduP and yqhD, and pEL175 for expressing atoB, hbd, crt, and ter. As shown in FIG. 2B, all PduP, except the one from P. gingivalis, enabled anaerobic growth rescue, and Bldh from Clostridium saccharoperbutylacetonicum was the positive control. The culture medium of these anaerobically grown cultures were analyzed for alcohol production (FIG. 2C) using gas chromatography (GC). PduP from L. brevis and K. pneumoniae produced more n-butanol than ethanol, while PduP from L. monocytigenes and S. enterica produced more ethanol than n-butanol at a ratio of 4:1. These results indicated that these PduP homologues catalyze the reduction of butyryl-CoA, in the desired and the non-native direction.

Substrate Specificity of PduP.

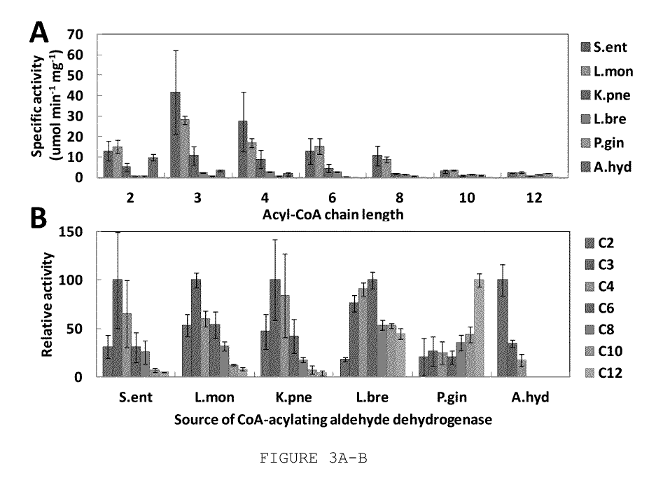

To characterize these PduP homologues, plasmids harboring individual pduP genes were constructed with a poly-His₆ tag (plasmids pCDF-pduP_ahyd, pCDF-pdup_kpne, pCDF-pdup_lbre, pCDF-pdup_lmon, pCDF-pdup_sent, and pCDF-pdup_pgin). PduP homologues were then purified under aerobic condition without cleaving off His tags. The activities of the enzymes were determined using acyl-CoA with different chain lengths, and the disappearance of NADH was monitored. As shown in FIG. 3A, PduP from S. enterica, L. monocytigenes, and K. pneumoniae exhibited higher activity than the other PduP homologues for carbon chain lengths ranging from 2 to 8. For butyryl-CoA reduction, S. enterica PduP was the most active (27±14 μmol/min/mg) under the assayed condition, followed by PduP from L. monocytigenes (17±2 μmol/min/mg), K. pneumoniae (8.9±4.6 μmol/min/mg), L. brevis (2.5±0.2 μmol/min/mg), A. hydrophilia (1.7±0.6 μmol/min/mg), and P. gingivalis (0.49±0.2 μmol/min/mg).

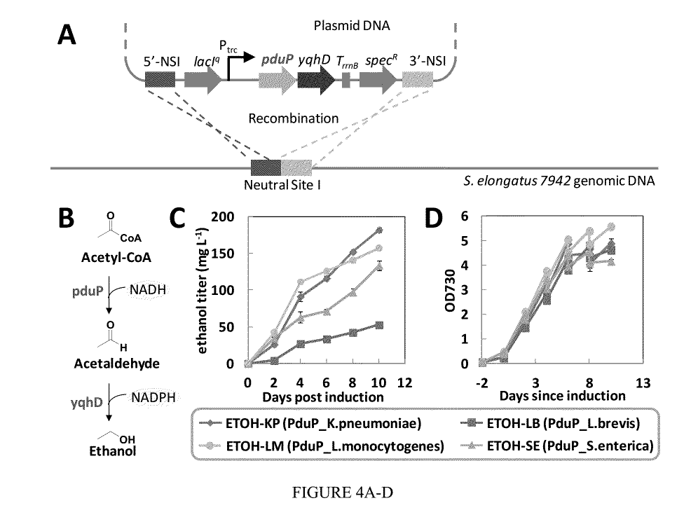

Specific activity was the highest for propionyl-CoA reduction compared to other acyl-CoAs for PduP from S. enterica, L. monocytigenes, and K. pneumoniae (FIG. 3B). This was expected since propionaldehyde is the natural substrate for PduP. Interestingly, PduP from L. brevis, P. gingivalis, and A. hydrophilia exhibited different substrate specificity. As shown in FIG. 4B, PduP from L. brevis and P gingivalis favors reduction of hexanoyl-CoA and dodecanoyl-CoA, respectively, over other substrate lengths. These results are particularly useful when the synthesis of longer chain aldehydes and alcohols are desired.

Kinetic Parameters of PduP.

The efficiency of these PduP homologues were assessed. Because these PduP enzymes act on both acetyl-CoA and butyryl-CoA, the ratio of their catalytic efficiency (k_{cat}/Kₘ) towards acetyl-CoA and butyryl-CoA is especially important for designing n-butanol production pathway. The kinetic parameters were measured for these PduP homologues by monitoring NADH oxidation over a range of acetyl-CoA and butyryl-CoA concentrations. The Kₘ and k_{cat} values for acetyl-CoA and butyryl-CoA are summarized in Table 2. Kinetic parameters for PduP from A. hydrophilia and Clostridium saccharoperbutylacetonicum were not determined due to a non-Michealis-Menten behavior and oxygen sensitivity, respectively. PduP from S. enterica showed the highest butyryl-CoA to acetyl-CoA catalytic efficiency ratio of 6.8, followed by PduP from K. pneumoniae (5.0), L. brevis (2.0), L. monocytigenes (1.4), and P. gingivalis (0.6). These results indicated that PduP from S. enterica, K. pneumoniae, L. brevis, and L. monocytigenes are suitable for co-expression with the CoA-dependent pathway in cyanobacteria for n-butanol production.

TABLE 2
Kinetic parameters (k_{cat} and Kₘ) of CoA-acylating aldehyde dehydrogenase

| | Acetyl-CoA | | | Butyryl-CoA | | | |
|---|---|---|---|---|---|---|---|
| Source | k_{cat} | Kₘ | k_{cat}/Kₘ | k_{cat} | Kₘ | k_{cat}/Kₘ | Ratio |
| Organism | (s⁻¹) | (μM) | (s⁻¹mM⁻¹) | (s⁻¹) | (μM) | (s⁻¹mM⁻¹) | C4:C2 |
| C. sac | n.d^{a} | | — | n.d^{a} | | — | — |
| A. hyd | n.d^{b} | | — | n.d^{b} | | — | — |
| K. pne | 4.63 ± 0.19 | 181.4 ± 25.1 | 25 | 7.02 ± 0.64 | 56.0 ± 25.9 | 125 | 5 |
| L. bre | 0.26 ± 0.10 | 76.0 ± 38.2 | 3.0 | 3.37 ± 0.19 | 534.3 ± 59.8 | 6.0 | 2 |
| L. mon | 11.33 ± 0.97 | 92.6 ± 20.1 | 122 | 11.99 ± 1.56 | 71.7 ± 20.3 | 167 | 1.4 |
| P. gin | 0.08 ± 0.01 | 75.9 ± 25.1 | 1.1 | 0.19 ± 0.02 | 256.0 ± 40.5 | 0.7 | 0.6 |
| S. ent | 14.81 ± 1.00 | 342.1 ± 94.8 | 43 | 25.38 ± 1.81 | 87.0 ± 24.8 | 292 | 6.8 |

^{a}not determined due to oxygen sensitivity
^{b}not determined due to non-Michaelis-Menten behavior

Co-Expression of PduP Enables Photosynthetic Ethanol Biosynthesis from Acetyl-CoA.

Next, the individual PduP enzymes were introduced with YqhD into S. elongatus PCC 7942 by homologous recombination (FIG. 4A) into Neutral Site (NS) I. Plasmids harboring individual pdup genes and yqhD were constructed under an IPTG inducible Trc promoter. These plasmids were then individually used to transform S. elongatus PCC 7942. The resulting S. elongatus strains ETOH-KP, ETOH-LB, ETOH-LM, and ETOH-SE (Table 3) express YqhD and PduP from K. pneumoniae, L. brevis, L. monocytigenes, and S. enterica, respectively. Expression of PduP and YqhD enabled photosynthetic ethanol production from acetyl-CoA (FIG. 4B). PduP reduces acetyl-CoA into acetaldehyde, which is then reduced to ethanol by YqhD.

TABLE 3
Cyanobacteria and E. coli strains used in this disclosure

| | | |
|---|---|---|
| Strain | Relevant genotypes | Reference |

Cyanobacteria Strains

| | | |
|---|---|---|
| PCC 7942 | Wild-type Synechococcus elongatus PCC 7942 | |
| EL9 | P_{Trc}::His-tagged ter integrated at NSI | Lan & Liao, 2011 |
| ETOH-KP | P_{Trc}::pduP_K.pneumoniae, yqhD integrated at NSI | This work |
| ETOH-LB | P_{Trc}::pduP_L.brevis, yqhD integrated at NSI | This work |
| ETOH-LM | P_{Trc}::pduP_L.monocytigenes, yqhD integrated at NSI | This work |
| ETOH-SE | P_{Trc}::pduP_S.enterica, yqhD integrated at NSI | This work |
| BUOH-LB | P_{Trc}::His-tagged ter integrated at NSI and | This work |
| | P_{LlacO1}::nphT7, pduP_L.brevis, yqhD, crt, hbd integrated at NSII | |
| BUOH-SE | P_{Trc}::His-tagged ter integrated at NSI and | This work |
| | P_{LlacO1}::nphT7, pduP_S.enterica, yqhD, crt, hbd integrated at NSII | |
| BUOH-LM | P_{Trc}::His-tagged ter integrated at NSI and | This work |
| | P_{LlacO1}::nphT7, pduP_L.monocytigenes, yqhD, crt, hbd integrated at NSII | |

E. coli strains

| | | |
|---|---|---|
| XL-1 blue | recA1 endA1 gyrA96 thi-1 hsdR17 supE44 relA1 lac [F′ proAB lacI^{q}ZΔM15 Tn10 (Tet^{R})] | Stratagene |
| JCL166 | BW25113 ΔldhA ΔadhE ΔfrdBC/F′ [traD36, proAB+, lacI^{q} ZΔM15 (Tet^{R})] | 22 |
| BL21(DE3) | F⁻ ompT hsdSB (rB⁻mB⁻) gal dcm rne131 (DE3) | In vitrogen |

nphT7 (Streptomyces sp. strain CL190), acetoacetyl-CoA synthase; phaB (R. Eutropha), acetoacetyl-CoA reductase; phaJ (A. caviae), (R)-specific enoyl-CoA hydratase; hbd (C. acetobutylicum), 3-hydroxybutyryl-CoA dehydrogenase; crt (C. acetobutylicum), crotonase; ter (T. denticola), Trans-2-enoyl-CoA reductase; yqhD (E. coli), NADP-dependent alcohol dehydrogenase; pduP (A. hydrophilia, K. pneumonia, L. brevis, L. monocytigenes, P. gingivalis, or S. enterica), CoA-acylating aldehyde dehydrogenase.

As shown in FIG. 4C, all strains expressing PduP homologues with YqhD demonstrated ethanol production. Strain ETOH-KP produced the highest amount of ethanol, reaching a cumulative titer of 182±4 mg/L after 10 days of cultivation with a peak productivity of 65 mg/L/d occurring between the Day 2 to 4. Strain ETOH-LM showed higher ethanol productivity than strain ETOH-KP for the first four days, after which production slowed down, reaching a cumulative titer of 157±3 mg/L after 10 days. ETOH-LM exhibited highest cell growth (FIG. 4D), reaching OD730 of 5.56, which is about 15% higher than strain ETOH-KP and 30% higher than the slowest growing strain ETOH-SE. Strains ETOH-SE and ETOH-LB each produced 133±7 mg/L and 53±4 mg/L of ethanol, respectively after 10 days. Note that photosynthetic ethanol productions demonstrated previously were all from non-oxidative decarboxylation of pyruvate catalyzed by pyruvate decarboxylase (Pdc), which is not oxygen sensitive. Thus, the results shown here represent the first demonstration of photosynthetic ethanol biosynthesis from acetyl-CoA, and further indicated that PduP and YqhD were functional under oxygenated photosynthetic condition, which is a major step for n-butanol production.

PduP Enables Efficient n-Butanol Synthesis in Cyanobacteria.

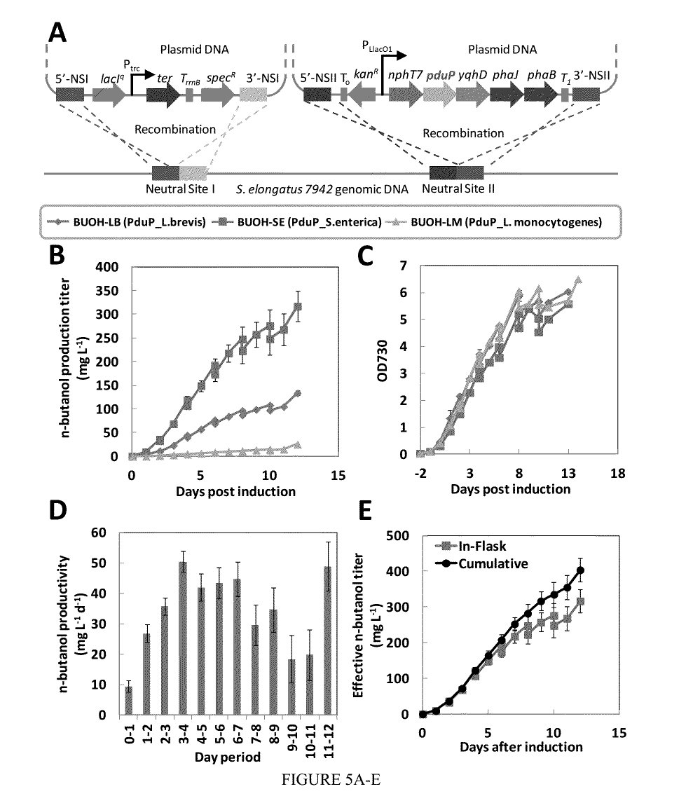

To construct an oxygen tolerant n-butanol producing pathway in cyanobacteria, PduP homologues were introduced with the other enzymes of the malonyl-CoA dependent pathway (NphT7, PhaB, PhaJ, Ter, and YqhD) into S. elongates (FIG. 1). Synthetic operons expressing NphT7, PhaB, PhaJ, YqhD, and each individual PduP homologue were constructed under control of an IPTG inducible PL_{1acO1} promoter (FIG. 5A). These plasmids were then used to recombine the synthetic operons individually into NSII of S. elongatus EL9 which expresses Ter at NSI. The resulting strains BUOH-LB, BUOH-LM, and BUOH-SE, expressing PduP from L. brevis, L. monocytigenes, and S. enterica, respectively, were genotypically verified by gene-specific PCR.

As shown in FIG. 5B, strain BUOH-SE produced the highest amount of n-butanol under photosynthetic conditions with an observed in-flask n-butanol titer of 317 mg/L in 12 days while BUOH-LB and BUOH-LM produced 134 mg/L and 25 mg/L, respectively. No detectable byproduct was observed in the culture medium, indicating the specificity of PduP for n-butanol production and the potential ease for product recovery. While these PduP homologues expressed by strains BUOH-LB, BUOH-LM, and BUOH-SE have higher catalytic efficiency towards butyryl-CoA than acetyl-CoA, it is interesting that no ethanol was detected in all three n-butanol producing strains. This result suggested that native S. elongatus acetyl-CoA carboxylase (Acc) and NphT7 efficiently channeled acetyl-CoA flux into n-butanol synthesis. Cell growth of strain BUOH-SE was slightly slower than that of the other strains (FIG. 5C). Daily productivity of strain BUOH-SE (FIG. 5D) increases with cell growth, reaching a maximum productivity of 51 mg/L/d between days 3 to 4. Daily productivity maintained around 40 mg/L/d until day 7, after which started to decline as culture entered stationary phase. The cumulative production titer (FIG. 5E) obtained from strain BUOH-SE was 404 mg/L in 12 days. Cumulative titer takes into account the dilutions made to cyanobacteria culture by feeding. Compared with S. elongatus expressing the malonyl-CoA dependent pathway using Bldh, strain BUOH-SE expressing PduP from S. enterica is a much more efficient n-butanol producer, exceeding the productivity of Bldh strain by 20 fold.

Oxygen sensitivity is one of the major obstacles for designing synthetic pathways in cyanobacteria. An ATP driven malonyl-CoA dependent pathway in cyanobacteria was previously constructed and was demonstrated to produce photosynthetic n-butanol. However, the oxygen sensitivity of Clostridium Bldh severely limited the productivity of n-butanol. The disclosure shows that in addition to the ATP driving force, oxygen tolerance was useful for constructing a functional CoA-dependent n-butanol synthesis pathway in cyanobacteria. Oxygen sensitivity of Clostridium pathway was solved by substituting Bldh with the oxygen tolerant PduP from S. enterica. The resulting S. elongatus strain produced 404 mg/L of n-butanol (FIG. 5E), comparable to or higher than the level achieved in many heterotrophs. This result represents the highest photosynthetic n-butanol production from CO₂ demonstrated to date.

Conversion between acyl-CoA and its corresponding aldehyde catalyzed by CoA-acylating aldehyde dehydrogenase is an important reaction occurring in both natural and synthetic pathways. For example, a synthetic 1,4-butanediol production pathway utilizes two CoA-acylating aldehyde dehydrogenases for converting succinyl-CoA to succinate semialdehyde and 4-hydroxybutyryl-CoA to 4-hydroxybutyraldehyde. Similarly, CO₂ fixation utilizing the 3-hydroxypropionate cycle also requires a CoA-acylating aldehyde dehydrogenases for converting malonyl-CoA to malonate semialdehyde. While CoA-acylating aldehyde dehydrogenases are ubiquitous, many of them from Clostridium are oxygen sensitive, prohibiting their use in photosynthetic microorganisms and other obligate aerobes. The oxygen tolerant PduP homologues that were characterized in this study are valuable for designing synthetic pathways requiring interconversion of acyl-CoA and its aldehyde. These PduPs have broad substrate specificity, ranging from C2 to C12. In particular, PduP from L. brevis and P. gingivalis have higher specific activity towards longer chain acyl-CoAs (FIG. 3B), thus suitable for the synthesis of higher alcohols such as 1-hexanol and 1-octanol.

Expression of PduP and YqhD alone enabled ethanol synthesis from acetyl-CoA. This pathway is different from the pyruvate dependent ethanol production demonstrated previously. Most of the PduP homologues characterized in this study exhibited a higher catalytic efficiency for butyryl-CoA over acetyl-CoA (Table 2). As a result, PduP selectively reacts with butyryl-CoA over acetyl-CoA when the CoA-dependent chain elongation is co-expressed. Co-expression of PduP and YqhD with the CoA-dependent chain elongation resulted in homo-butanol production in S. elongatus with no traces of ethanol or other detectable by-products in the culture medium, which is favorable for downstream product recovery.

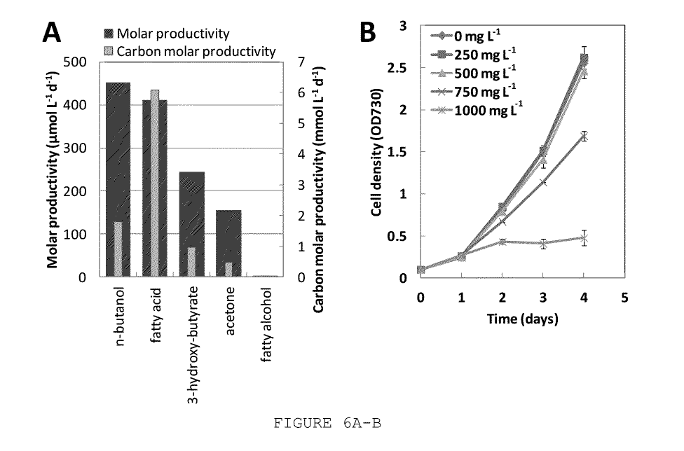

Utilizing cyanobacteria as a catalyst to reduce CO₂ into usable chemicals is an attractive direction for sustainable chemistry and CO₂ mitigation. Specifically, S. elongatus PCC 7942 is a suitable host for n-butanol production because it does not have (3-oxidation, thus cannot reuptake and metabolize n-butanol and preventing yield loss. The n-butanol productivity achieved in this study compares favorably to that of other acetyl-CoA derived products demonstrated in literature (FIG. 6A). With the exception of fatty acid production, the n-butanol productivity demonstrated here also compares favorably on the basis of carbon molar productivity, which is defined as molar productivity per carbon in the compound. However, several obstacles have to be overcome in order for it to be industrially feasible. One such challenge is the toxicity of n-butanol. As shown in FIG. 6B, at 750 mg/L of n-butanol in culture medium, S. elongatus strain BUOH-SE exhibited visible growth retardation. At 1 g/L of n-butanol, cell growth is inhibited. One approach to avoid product toxicity is to remove product in situ. Alternatively, directed evolution in combination with rational designs aided by systems analysis may be useful to develop a higher butanol-tolerant host. Combining efforts of pathway engineering demonstrated here and future systems optimization, photosynthetic production of n-butanol is promising.

The following table shows calculation for converting productivity reported in the literature into molar productivity

| | | | | | Molar |
|---|---|---|---|---|---|
| | Titer | Time | Productivity | Molar mass | productivity |
| Units | mg/L | days | mg/L/d | mmol/mg | μmol/L/d |
| n-butanol | 402 | 12 | 33.5 | 74.12 | 452 |
| fatty acid | 197 | 2 | 98.5 | 172-284 | 411^{a} |
| 3-hydroxy- | 533.4 | 21 | 25.4 | 104.1 | 244 |
| butyrate | | | | | |
| acetone | 36 | 4 | 9 | 58.08 | 155 |
| fatty alcohol | 0.20044 | 18 | 0.011 | 242-270 | 0.0422^{b} |

The following table calculates the molar productivity of each fatty acid

| | | | | Molar |
|---|---|---|---|---|
| | | productivity | molar mass | productivity |
| Fatty acid | percentage | mg/L/d | mmol/mg | μmol/L/d |
| 10 | 0.6 | 0.6 | 172.3 | 3 |
| 12 | 19.9 | 19.6 | 200.3 | 98 |
| 14 | 20.9 | 20.6 | 228.4 | 90 |
| 16 | 43.3 | 42.7 | 256.4 | 166 |
| 18:3 | 1.4 | 1.4 | 278.4 | 5 |
| 18:2 | 1.1 | 1.1 | 280.5 | 4 |
| 18:1 | 1.5 | 1.5 | 282.5 | 5 |
| 18 | 11.3 | 11.1 | 284.5 | 39 |
| Total | 100 | | | 411 |

The following table calculates the molar productivity of each fatty alcohol.

| | | | | Molar |
|---|---|---|---|---|
| | | productivity | molar mass | productivity |
| Fatty alcohol | percentage | mg/L/d | mmol/mg | μmol/L/d |
| 16 | 20 | 0.0022 | 242.0 | 0.00920 |
| 18 | 80 | 0.0089 | 270.0 | 0.03299 |
| Total | 100 | 0.0111 | | 0.0422 |

^{a}Fatty acids of different chain length and saturation were produced.
^{b}Fatty alcohols of different chain length and saturation were produced.
*Note productivity of 0.0111 mg/L/d was calculated based on the reported production of 200 μg/L over 18 days.

The following table shows calculation for converting productivity reported in the literature into carbon molar productivity

| | Molar | | Carbon molar |
|---|---|---|---|
| | productivity | Numbers of | productivity |
| Units | μmol/L/d | carbons | mmol/L/d |
| n-butanol | 452 | 4 | 1.8 |
| fatty acid | 411 | 10-18 | 6.1 |
| 3-hydroxy- | 244 | 4 | 1.0 |
| butyrate | | | |
| acetone | 155 | 3 | 0.5 |
| fatty alcohol | 0.0422 | 16-18 | 0.0024 |

A number of embodiments of the invention have been described. Nevertheless, it will be understood that various modifications may be made without departing from the spirit and scope of the invention. Accordingly, other embodiments are within the scope of the following claims.

1. A recombinant microorganism the converts a carbon source to a biofuel alcohol comprising a recombinantly expressed oxygen tolerant CoA-acylating aldehyde dehydrogenase (PduP) comprising a sequence that is at least 85% identical to SEQ ID NO:34.

2. The recombinant microorganism of claim 1, wherein the microorganism is a photoautotroph or photoheterotroph microorganism.

3. The recombinant microorganism of claim 2, wherein the microorganism produces a metabolite in the production of n-butanol and/or producing n-butanol wherein the metabolite and/or n-butanol is produced through a malonyl-CoA dependent pathway comprising an oxygen tolerant CoA-acylating aldehyde dehydrogenase.

4. The recombinant photoautotroph or photoheterotroph microorganism of claim 2, wherein the organism comprises expression or elevated expression of an enzyme that converts acetyl-CoA to malonyl-CoA, malonyl-CoA to Acetoacetyl-CoA, and at least one enzyme that converts acetoacetyl-CoA to (R)- or (S)-3-hydroxybutyryl-CoA, (R)- or (S)-3-hydroxybutyryl-CoA to crotonyl-CoA, crotonyl-CoA to butyryl-CoA, butyryl-CoA to butyraldehyde and butyraldehyde to 1-butanol.

5. The recombinant microorganism of claim 3, wherein the microorganism comprises a metabolic pathway for the production of 1-butanol that is an NADPH dependent pathway.

6. The recombinant microorganism of claim 3, wherein the photoautotrophic or photoheterotrophic microorganism is engineered to express or overexpress one or more polypeptides that convert acetyl-CoA to malonyl-CoA and malonyl-CoA to Acetoacetyl-CoA.

7. The recombinant microorganism of claim 6, wherein the one or more polypeptides comprises a nphT7 polypeptide comprising at least 90% identity to SEQ ID NO:18 and having acetoacetyl-CoA synthase activity.

8. The recombinant microorganism of claim 1, wherein the recombinant microorganism is engineered to express an acetyl-CoA carboxylase.

9. The recombinant microorganism of claim 8, wherein the acetyl-CoA carboxylase comprises a sequence that is at least 90% identical to SEQ ID NO:2 and has acetyl-CoA carboxylase activity.

10. The recombinant microorganism of claim 1, wherein the microorganism further expresses or overexpresses one or more enzymes that carries out a metabolic function selected from the group consisting of (a) converting acetoacetyl-CoA to (R)-3-hydroxybutyryl-CoA, (b) converting acetoacetyl-CoA to (S)-3-hydroxybutyryl-CoA, (c) converting (R)-3-hydroxybutyryl-CoA to crotonyl-CoA, (d) converting (S)-3-hydroxybutyryl-CoA to crotonyl-CoA, (e) converting crotonyl-CoA to butyryl-CoA, and (f) converting butyryl-CoA to butyraldehyde and butyraldehyde to 1-butanol.

11. The recombinant microorganism of claim 10, wherein the recombinant microorganism comprises an NADPH dependent metabolic pathway that converts (i) acetyl-CoA to malonyl-CoA, (ii) malonyl-CoA to acetoacetyl-CoA, (iii) acetoacetyl-CoA to (R)-3-hydroxybutyryl-CoA, (iv) (R)-3-hydroxybutyryl-CoA to crotonyl-CoA, (v) crotonyl-CoA to butyryl-CoA, (vi) butyryl-CoA to butyraldehyde, and (vii) butyraldehyde to 1-butanol.

12. The recombinant microorganism of claim 10, wherein the recombinant microorganism comprises an NADPH dependent metabolic pathway that converts (i) acetyl-CoA to acetoacetyl-CoA, (ii) acetoacetyl-CoA to (R)-3-hydroxybutyryl-CoA, (iii) (R)-3-hydroxybutyryl-CoA to crotonyl-CoA, (iv) crotonyl-CoA to butyryl-CoA, (v) butyryl-CoA to butyraldehyde, and (vi) butyraldehyde to 1-butanol.

13. The recombinant microorganism of claim 10, wherein the microorganism is a photoautotrophic or photoheterotrophic organism that is engineered to express or overexpress an acetyl-CoA carboxylase and an acetoacetyl-CoA synthase.

14. The recombinant microorganism of claim 13, wherein the microorganism further expresses or overexpresses one or more enzymes selected from the group consisting of (a) acetoacetyl-CoA reductase, (b) enoyl-CoA hydratase, (c) crotonyl-CoA reductase, and (d) an oxygen tolerant CoA-acylating aldehyde dehydrogenase.

15. The recombinant microorganism of claim 10, wherein the microorganism comprises a photoautotrophic or photoheterotrophic organism that is engineered to express or overexpress an acetyl-CoA carboxylase and an acetoacetyl-CoA synthase and further expresses or overexpresses one or more enzymes selected from the group consisting of (a) acetoacetyl-CoA reductase, (b) enoyl-CoA hydratase, (c) trans-2-enoyl-CoA reductase, and (d) an oxygen tolerant CoA-acylating aldehyde dehydrogenase.

16. The recombinant microorganism of claim 10, wherein the microorganism comprises a photoautotrophic or photoheterotrophic organism that is engineered to express or overexpress an acetyl-CoA carboxylase and an acetoacetyl-CoA synthase and further expresses or overexpresses one or more enzymes selected from the group consisting of (a) acetoacetyl-CoA reductase, (b) enoyl-CoA hydratase, (c) trans-2-enoyl-CoA reductase, and (d) oxygen tolerant CoA-acylating aldehyde dehydrogenase and 1,3-propanediol dehydrogenase.

17. The recombinant microorganism of claim 10, wherein the microorganism comprises a photoautotrophic or photoheterotrophic organism that is engineered to express or overexpress an acetyl-CoA carboxylase and an acetoacetyl-CoA synthase and one or more enzymes selected from the group consisting of (a) hydroxybutyryl CoA dehydrogenase, (b) crotonase, (c) trans-2-enoyl-CoA reductase, and (d) oxygen tolerant CoA-acylating aldehyde dehydrogenase and 1,3-propanediol dehydrogenase.

18. The recombinant microorganism of claim 3, wherein the microorganism comprises a photoautotrophic or photoheterotrophic organism and includes the expression of at least one heterologous, or the over expression of at least one endogenous, target enzyme from the group consisting of an enzyme that converts (i) acetyl-CoA to malonyl-CoA, (ii) malonyl-CoA to Acetoacetyl-CoA, (iii) acetoacetyl-CoA to (R)- or (S)-3-hydroxybutyryl-CoA, (iv) (R)- or (S)-3-hydroxybutyryl-CoA to crotonyl-CoA, (v) crotonyl-CoA to butyryl-CoA, (vi) butyryl-CoA to butyraldehyde and (vi) butyraldehyde to 1-butanol.

19. The recombinant microorganism of claim 1, wherein the microorganism comprises a photoautotrophic or photoheterotrophic organism that comprises a reduction, disruption or knockout of at least one gene encoding an enzyme that competes with a metabolite necessary for the production of a desired higher alcohol product or which produces an unwanted product.

20. The recombinant microorganism of claim 19, wherein the microorganism comprises a photoautotrophic or photoheterotrophic organism that is engineered to disrupt, delete or knockout one or more genes encoding a polypeptide or protein selected from the group consisting of: (i) an enzyme that catalyzes the NADH-dependent conversion of pyruvate to D-lactate; (ii) an enzyme that promotes catalysis of fumarate and succinate interconversion; (iii) an oxygen transcription regulator; and (iv) an enzyme that catalyzes the conversion of acetyl-coA to acetyl-phosphate.

21. The recombinant microorganism of claim 20, comprises a disruption, deletion or knockout of a combination of an alcohol/acetoaldehyde dehydrogenase and one or more of (i)-(iv).

22. The recombinant microorganism of claim 1, wherein the microorganism is engineered to express one or more subunits of acetyl-coA carboxylase (AccABCD) that converts acetyl-CoA to malonyl-CoA.

23. The recombinant microorganism of claim 1, wherein the microorganism is engineered to express of over express one or more genes selected from the group consisting of nphT7, phaB, phaJ, ter, pdup, and yqhD, and wherein the microorganism produces n-butanol.

24. The recombinant microorganism of claim 23, further comprising expressing or over expressing AccABCD.

25. The recombinant microorganism of claim 1, wherein the microorganism expresses a polypeptide having at least 90-100% identity to SEQ ID NO:2 and having acetyl-coA carboxylase activity.

26. The recombinant microorganism of claim 1, wherein the microorganism expresses a polypeptide having at least 90-100% identity to SEQ ID NO:18 and has acetoacetyl-CoA synthase activity.

27. The recombinant microorganism of claim 1, wherein the microorganism expresses a polypeptide having at least 90-100% identity to SEQ ID NO:30 and has acetoacetyl-CoA reductase activity.

28. The recombinant microorganism of claim 1, wherein the microorganism expresses a polypeptide having at least 90-100% identity to SEQ ID NO:28 and has R-specific crotonase activity.

29. The recombinant microorganism of claim 1, wherein the microorganism expresses a polypeptide having at least 90-100% identity to SEQ ID NO:23, 24, 25, or 26 and has trans-enoyl-CoA reductase activity.

30. The recombinant microorganism of claim 1, wherein the microorganism expresses a polypeptide having at least 90-100% identity to SEQ ID NO:34 and has oxygen tolerant CoA-acylating aldehyde dehydrogenase.

31. The recombinant microorganism of claim 1, wherein the microorganism expresses a polypeptide having at least 90-100% identity to SEQ ID NO:32 and has NADPH dependent alcohol dehydrogenase activity.

32. The recombinant microorganism of claim 1, wherein the microorganism comprises an expression profile selected from the group consisting of:
(a) AccABCD, nphT7, PhaB, PhaJ, Ter, PduP, and YqhD,
(b) nphT7, PhaB, PhaJ, Ter, PduP, and YqhD,
(c) AccABCD, nphT7, PhaB, PhaJ, ccr, PduP, and YqhD,
(d) nphT7, PhaB, PhaJ, ccr, PduP, and YqhD,
(e) AccABCD, nphT7, hbd, crt, Ter, PduP, and YqhD, and
(f) nphT7, hbd, crt, Ter, PduP, and YqhD.

33. A method for producing an alcohol, the method comprising:
a) providing a recombinant photoautotroph or photoheterotrophic microorganism of claim 32;
b) culturing the microorganism(s) of (a) in the presence of CO₂ under conditions suitable for the conversion of the substrate to an alcohol; and
c) purifying the alcohol.

34. A culture comprising a recombinant microorganism of claim 1.

35. A recombinant vector comprising any two or more genes selected from the group consisting of AccA, AccB, AccC, AccD, nphT7, PhaB, PhaJ, Ter, PduP, and YqhD or any homologs of each of the foregoing.

36-37. (canceled)


SEQUENCE LISTING (80 sequences)

SEQ ID NO: 1 - DNA, 951 bp, Pseudomonas aeruginosa
  CDS (1)..(951)
atgaacccgaactttcttgatttcgaacagccgatcgccgacctgcaagccaagatcgaagagctgcgcctggtgggcaacgacaatgcgctgaacatcagcgacgaaatctcgcgtctgcaggacaagagcaaggcgctcaccgaaaacatcttcggcaatctgtccagttggcagatcgcccagctcgcgcgccatcccaagcgtccctataccctcgactacatcggctacctgttcagcgatttcgaggaactgcacggcgaccggcatttcgccgacgacccggcgatcgtcggcggcgttgcccgcctcgacggttccccggtgatggtcatcggccaccagaagggccgcgaagtccgtgagaaggtccggcgcaacttcggcatgccgcgtccggaaggctatcgcaaggcctgccgcctgatggaaatggccgaacgcttcaagatgccgatcctcaccttcatcgacacgcccggcgcctacccggggatcgatgccgaggaacgcggccagagcgaggcgatcgcctggaacctgcgggtgatggcgcgactgaagacgccgatcatcgccaccgtgatcggcgagggcggttccggcggcgcgctggccatcggtgtctgcgaccagttgaacatgctgcaatactccacctattcggtgatctcgccggaaggctgcgcctccatcctctggaagaccgccgagaaggcgccggaagccgccgaggccatgggcatcaccgccgagcgcctgaaaggcctgggcatcgtcgacaaggtcatcgacgaaccgctgggcggcgcccatcgcgatccggcgagcatggccgaatcgatccgtggcgaactgctggcgcaactgaagatgctccagggcctggaaatgggtgagttgctggagcgtcgttacgaccgcctgatgagctacggcgcgccgtaa

SEQ ID NO: 2 - protein, 316 aa, Pseudomonas aeruginosa
MNPNFLDFEQPIADLQAKIEELRLVGNDNALNISDEISRLQDKSKALTENIFGNLSSWQIAQLARHPKRPYTLDYIGYLFSDFEELHGDRHFADDPAIVGGVARLDGSPVMVIGHQKGREVREKVRRNFGMPRPEGYRKACRLMEMAERFKMPILTFIDTPGAYPGIDAEERGQSEAIAWNLRVMARLKTPIIATVIGEGGSGGALAIGVCDQLNMLQYSTYSVISPEGCASILWKTAEKAPEAAEAMGITAERLKGLGIVDKVIDEPLGGAHRDPASMAESIRGELLAQLKMLQGLEMGELLERRYDRLMSYGAP

SEQ ID NO: 3 - DNA, 1344 bp, Streptomyces coelicolor
  CDS (1)..(1344)
gtgaccgtgaaggacatcctggacgcgatccagtcgcccgactccacgccggccgacatcgccgcactgccgctccccgagtcgtaccgcgcgatcaccgtgcacaaggacgagaccgagatgttcgcgggcctcgagacccgcgacaaggacccccgcaagtcgatccacctggacgacgtgccggtgcccgagctgggccccggcgaggccctggtggccgtcatggcctcctcggtcaactacaactcggtgtggacctcgatcttcgagccgctgtccaccttcgggttcctggagcgctacggccgggtcagcgacctcgccaagcggcacgacctgccgtaccacgtcatcggctccgacctcgccggtgtcgtcctgcgcaccggtccgggcgtcaacgcctggcaggcgggcgacgaggtcgtcgcgcactgcctctccgtcgagctggagtcctccgacggccacaacgacacgatgctcgaccccgagcagcgcatctggggcttcgagaccaacttcggcggcctcgcggagatcgcgctggtcaagtccaaccagctgatgccgaagccggaccacctgagctgggaggaggccgccgctcccggcctggtcaactccaccgcgtaccgccagctcgtctcccgcaacggcgccggcatgaagcagggcgacaacgtgctcatctggggcgcgagcggcggactcggctcgtacgccacccagttcgccctcgccggcggcgccaacccgatctgcgtcgtctcctcgccgcagaaggcggagatctgccgcgcgatgggcgccgaggcgatcatcgaccgcaacgccgagggctaccggttctggaaggacgagaacacccaggacccgaaggagtggaagcgcttcggcaagcgcatccgcgaactgaccggcggcgaggacatcgacatcgtcttcgagcaccccggccgcgagaccttcggcgcctccgtcttcgtcacccgcaagggcggcaccatcaccacctgcgcctcgacctcgggctacatgcacgagtacgacaaccgctacctgtggatgtccctgaagcgcatcatcggctcgcacttcgccaactaccgcgaggcctgggaggccaaccgcctcatcgccaagggcaggatccaccccacgctctccaaggtgtactccctcgaggacaccggccaggccgcctacgacgtccaccgcaacctccaccagggcaaggtcggcgtgctgtgcctggcgcccgaggagggcctgggcgtgcgcgaccgggagaagcgcgcgcagcacctcgacgccatcaaccgcttccggaacatctga

SEQ ID NO: 4 - protein, 447 aa, Streptomyces coelicolor
VTVKDILDAIQSPDSTPADIAALPLPESYRAITVHKDETEMFAGLETRDKDPRKSIHLDDVPVPELGPGEALVAVMASSVNYNSVWTSIFEPLSTFGFLERYGRVSDLAKRHDLPYHVIGSDLAGVVLRTGPGVNAWQAGDEVVAHCLSVELESSDGHNDTMLDPEQRIWGFETNFGGLAEIALVKSNQLMPKPDHLSWEEAAAPGLVNSTAYRQLVSRNGAGMKQGDNVLIWGASGGLGSYATQFALAGGANPICVVSSPQKAEICRAMGAEAIIDRNAEGYRFWKDENTQDPKEWKRFGKRIRELTGGEDIDIVFEHPGRETFGASVFVTRKGGTITTCASTSGYMHEYDNRYLWMSLKRIIGSHFANYREAWEANRLIAKGRIHPTLSKVYSLEDTGQAAYDVHRNLHQGKVGVLCLAPEEGLGVRDREKRAQHLDAINRFRNI

SEQ ID NO: 5 - DNA, 1206 bp, Pseudomonas syringae
  CDS (1)..(1206)
atgaatcaagcactgactgaaaccatgcaggcctttctgatccgccccgagcgctatggcgaaccgcagcaggccatccagctcgaacaggtccagatccccaccctgggtccgcatcaggtcctcatcgaagtgatggcagccggactcaactacaacaacgtctgggccgcccagggtaagccggtggacatcatcgccgcgcggcgcaagcggaaccgtgacgccgaacccttccacatcggaggctcggaagcctccggttacgtgaaagccgtgggcgacgctgtcacccacgtcaaggtgggcgataccgtggtggtgtcctgctcggtctacgacgccacggccatcgaatcgcgcgtcgcccccgaccccatgttctgcagcaaccaggaaatctacggctacgagaccagctacggctccttcgccgaatacaccctcgtcgaagactaccaatgcttcccaaaaccaaagttcctgagctgggaggaaagtgccaccctgatgctcaatggtccgaccgcctacaagcagctcacgcattgggcacccaataccgtcaagcctggagacgcagtcctgatctggggcgcggcaggtggcctgggctctatgtctatccagttgacccgcgcgctcggggggctgccggtggccgtggtgtccagtccagacaggggccgctacgcctgcgaactcggcgccgtggggtacttgctcagaaccgactatccgcacctgggacgtctgccggacttgaactccgacgctcacagcgcctggaccaaaagcttcgcgagtttccgtcgcgacttcttcatgacgctggggaaaaaggagctgcccaaagtggtgatcgagcactccggccaagccaccttccccacctcgctgcagatctgcgaccgctccggcatggtggtcatcgtgggtggcacgtccggctacaactgcgacttcgatgtccgccacctgtggatgcaccagaagcgcatccagggctcccactacgccaacatccgcgagtgccaggaattcctgcaactagtcgaacaacgccgggtagtgccgaccctgaacaccctctatcgcttcgaggagacacctagggcgcatcaggcgctactgagtggagaagtcgtaggcaatgccgccgtgctggtcaaggccgagcgacccggcctaggggtcggttgttga

SEQ ID NO: 6 - protein, 401 aa, Pseudomonas syringae
MNQALTETMQAFLIRPERYGEPQQAIQLEQVQIPTLGPHQVLIEVMAAGLNYNNVWAAQGKPVDIIAARRKRNRDAEPFHIGGSEASGYVKAVGDAVTHVKVGDTVVVSCSVYDATAIESRVAPDPMFCSNQEIYGYETSYGSFAEYTLVEDYQCFPKPKFLSWEESATLMLNGPTAYKQLTHWAPNTVKPGDAVLIWGAAGGLGSMSIQLTRALGGLPVAVVSSPDRGRYACELGAVGYLLRTDYPHLGRLPDLNSDAHSAWTKSFASFRRDFFMTLGKKELPKVVIEHSGQATFPTSLQICDRSGMVVIVGGTSGYNCDFDVRHLWMHQKRIQGSHYANIRECQEFLQLVEQRRVVPTLNTLYRFEETPRAHQALLSGEVVGNAAVLVKAERPGLGVGC

SEQ ID NO: 7 - DNA, 1293 bp, Rhodobacter sphaerides
  CDS (1)..(1293)
atggccctcgacgtgcagagcgatatcgtcgcctacgacgcgcccaagaaggacctctacgagatcggcgagatgccgcctctcggccatgtgccgaaggagatgtatgcttgggccatccggcgcgagcgtcatggcgagccggatcaggccatgcagatcgaggtggtcgagacgccctcgatcgacagccacgaggtgctcgttctcgtgatggcggcgggcgtgaactacaacggcatctgggccggcctcggcgtgcccgtctcgccgttcgacggtcacaagcagccctatcacatcgcgggctccgacgcgtcgggcatcgtctgggcggtgggcgacaaggtcaagcgctggaaggtgggcgacgaggtcgtgatccactgcaaccaggacgacggcgacgacgaggaatgcaacggcggcgacccgatgttctcgcccacccagcggatctggggctacgagacgccggacggctccttcgcccagttcacccgcgtgcaggcgcagcagctgatgaagcgtccgaagcacctgacctgggaagaggcggcctgctacacgctgaccctcgccaccgcctaccggatgctcttcggccacaagccgcacgacctgaagccggggcagaacgtgctggtctggggcgcctcgggcggcctcggctcctacgcgatccagctcatcaacacggcgggcgccaatgccatcggcgtcatctcagaggaagacaagcgcgacttcgtcatggggctgggcgccaagggcgtcatcaaccgcaaggacttcaagtgctggggccagctgcccaaggtgaactcgcccgaatataacgagtggctgaaggaggcgcgcaagttcggcaaggccatctgggacatcaccggcaagggcatcaacgtcgacatggtgttcgaacatccgggcgaggcgaccttcccggtctcgtcgctggtggtgaagaagggcggcatggtcgtgatctgcgcgggcaccaccggcttcaactgcaccttcgacgtccgctacatgtggatgcaccagaagcgcctgcagggcagccatttcgccaacctcaagcaggcctccgcggccaaccagctgatgatcgagcgccgcctcgatccctgcatgtccgaggtcttcccctgggccgagatcccggctgcccatacgaagatgtataagaaccagcacaagcccggcaacatggcggtgctggtgcaggccccgcgcacggggttgcgcaccttcgccgacgtgctcgaggccggccgcaaggcctga

SEQ ID NO: 8 - protein, 430 aa, Rhodobacter sphaerides
MALDVQSDIVAYDAPKKDLYEIGEMPPLGHVPKEMYAWAIRRERHGEPDQAMQIEVVETPSIDSHEVLVLVMAAGVNYNGIWAGLGVPVSPFDGHKQPYHIAGSDASGIVWAVGDKVKRWKVGDEVVIHCNQDDGDDEECNGGDPMFSPTQRIWGYETPDGSFAQFTRVQAQQLMKRPKHLTWEEAACYTLTLATAYRMLFGHKPHDLKPGQNVLVWGASGGLGSYAIQLINTAGANAIGVISEEDKRDFVMGLGAKGVINRKDFKCWGQLPKVNSPEYNEWLKEARKFGKAIWDITGKGINVDMVFEHPGEATFPVSSLVVKKGGMVVICAGTTGFNCTFDVRYMWMHQKRLQGSHFANLKQASAANQLMIERRLDPCMSEVFPWAEIPAAHTKMYKNQHKPGNMAVLVQAPRTGLRTFADVLEAGRKA

SEQ ID NO: 9 - DNA, 1284 bp, Rhodospirillum rubrum
  CDS (1)..(1284)
atgaccacgtcggcggaagtcatagaactcaatcccggcactggccggaaggatctttacgaactcggtgaaattccgccgctcggccacgttcccaagtctatgtacgcctgggtcatccgccgggatcgccatggcgaacccgagaagtctttccaggttgaagtcgttgaaacgccaactcttgacagccacgacgtcttggtgatggtgatggcggccggcgtcaactacaacgggatctgggccggattgggccagccgatcagcgttttcgactcgcataaggccgcttatcacatcgccggttcggatgcggcgggcatcgtctgggccgtcggcgccaaggtcaagcgctggaaggtcggcgacgaggtggtcgtccactgcaatcagaccgacggcgacgacgaggaatgcaatggtggcgatccgatgttctcgccgacccagcgcatctggggctatgagacccccgatggctccttcgcccagttcacccgcgtgcagtcccagcaggtgatggcccgtccgcgccatctgacctgggaggaaagtgccagctacgtgctggttctggccaccgcctatcgcatgctgttcggccaccgcccccatgtgctgcgcccgggtcacaacgtgctgatctggggcgcctcgggcggcctgggatcgatggcgatccagctgtgcgccacggcgggcgccaatgccatcggcgtcatctccgatgagaccaagcgcgatttcgtcatgagcctgggcgccaagggcgtgatcaaccgcaaggatttcaattgctggggccaattgcccacggtcaatggcgagggcttcgacgcctatatgaaagaggtgcgcaagttcggcaaggcgatctgggacatcaccggcaagggcaacgacgttgatttcgtgttcgaacatccgggcgagcagaccttcccggtctcgtgcaatgtggtcaagcgcggtggcatggtggtgttttgcgccggcaccaccggcttcaacctgaccttcgacgcccgctttgtgtggatgcgccagaagcgcattcagggcagccacttcgccaatctgctccaggcctcgcaagccaaccagttggtcatcgagcggcggatcgatccgtgcatgagcgaagtgttttcctgggacgatattcccaaggcccacaccaagatgtggaagaatcagcataagccggggaatatggcggtgctggtccaggcccatcgcccgggccgccgcaccttggaggattgccgagaggaagggtga

SEQ ID NO: 10 - protein, 427 aa, Rhodospirillum rubrum
MTTSAEVIELNPGTGRKDLYELGEIPPLGHVPKSMYAWVIRRDRHGEPEKSFQVEVVETPTLDSHDVLVMVMAAGVNYNGIWAGLGQPISVFDSHKAAYHIAGSDAAGIVWAVGAKVKRWKVGDEVVVHCNQTDGDDEECNGGDPMFSPTQRIWGYETPDGSFAQFTRVQSQQVMARPRHLTWEESASYVLVLATAYRMLFGHRPHVLRPGHNVLIWGASGGLGSMAIQLCATAGANAIGVISDETKRDFVMSLGAKGVINRKDFNCWGQLPTVNGEGFDAYMKEVRKFGKAIWDITGKGNDVDFVFEHPGEQTFPVSCNVVKRGGMVVFCAGTTGFNLTFDARFVWMRQKRIQGSHFANLLQASQANQLVIERRIDPCMSEVFSWDDIPKAHTKMWKNQHKPGNMAVLVQAHRPGRRTLEDCREEG

SEQ ID NO: 11 - DNA, 1338 bp, Streptomyces avermitilis
  CDS (1)..(1338)
gtgaaggaaatcctggacgcgattcagtcccagacggccacgtctgccgacttcgccgcactgccgctccccgactcgtaccgcgcgatcaccgtgcacaaggacgagacggagatgttcgccgggctcagcacccgcgacaaggacccccgcaagtcgatccacctggacgacgtgccggtgccggagctcggccccggcgaggccctggtggccgtcatggcgtcctccgtgaactacaactcggtctggacgtcgatcttcgagccggtgtcgaccttcaacttcctggagcgctacgggcggctcagcgatctcagcaagcgccacgacctgccgtaccacatcatcggttctgacctcgcgggcgtcgtcctgcgcaccggcccgggagtcaactcctggaagcccggcgacgaggtcgtcgcgcactgtctctcggtcgagctggagtcgtccgacggccacaacgacacgatgctcgaccccgagcagcgcatctggggcttcgagaccaacttcggcgggctcgccgagatcgcgctcgtcaagtccaaccagctgatgccgaagccggaccacctcagctgggaggaggccgccgctccgggcctggtgaactcgaccgcgtaccggcagctcgtctcccgcaacggcgccggcatgaagcagggcgacaacgtcctcatctggggcgcgagcggtggactgggctcgtacgccacgcagttcgcgctcgccggcggcgccaacccgatctgcgtcgtctccagcgagcagaaggcggacatctgccgctcgatgggcgccgaggcgatcatcgaccgcaacgccgagggctacaagttctggaaggacgagaccacccaggacccgaaggagtggaagcgcttcggcaagcgcatccgcgagttcaccggcggcgaggacatcgacatcgtcttcgagcaccccggccgcgagaccttcggcgcctcggtctacgtcacccgcaagggcggcaccatcaccacctgcgcctcgacctcgggctacatgcacgagtacgacaaccgctacctgtggatgtcgctgaagcggatcatcggctcgcacttcgcgaactaccgcgaggcctgggaggccaaccgcctcgtcgccaagggcaagatccaccccacgctctccaaggtctactccctggaggacaccgggcaggccgcctacgacgtgcaccgcaacctccaccagggcaaggtcggcgtgctcgccctcgcgccccgcgagggcctgggcgtgcgcgacgaggagaagcgcgcgcagcacatcgacgccatcaaccgcttccggaacatctga

SEQ ID NO: 12 - protein, 445 aa, Streptomyces avermitilis
VKEILDAIQSQTATSADFAALPLPDSYRAITVHKDETEMFAGLSTRDKDPRKSIHLDDVPVPELGPGEALVAVMASSVNYNSVWTSIFEPVSTFNFLERYGRLSDLSKRHDLPYHIIGSDLAGVVLRTGPGVNSWKPGDEVVAHCLSVELESSDGHNDTMLDPEQRIWGFETNFGGLAEIALVKSNQLMPKPDHLSWEEAAAPGLVNSTAYRQLVSRNGAGMKQGDNVLIWGASGGLGSYATQFALAGGANPICVVSSEQKADICRSMGAEAIIDRNAEGYKFWKDETTQDPKEWKRFGKRIREFTGGEDIDIVFEHPGRETFGASVYVTRKGGTITTCASTSGYMHEYDNRYLWMSLKRIIGSHFANYREAWEANRLVAKGKIHPTLSKVYSLEDTGQAAYDVHRNLHQGKVGVLALAPREGLGVRDEEKRAQHIDAINRFRNI

SEQ ID NO: 13 - DNA, 1287 bp, Silicibacter pomeroyi
  CDS (1)..(1287)
atggctttggacaccgacagcggtatcgcgtcctacgcggcgcccgagaaagacctctatgagatgggtgaaatccccccgatgggattcgtgcccaagaagatgtatgcgtgggcgatccgcaaagagcgccacggtgatcccgataccgcgatgcaggtcgaagtggttgacgtgccgacgctcgacagccacgaggtgctggttctggtgatggccgctggcgtcaactacaatggcgtctgggcctccaaaggtgttccgatttcccccttcgatggccacggacagccctatcacatcgccggttccgatgcttcgggtatcgtctgggccgtgggggacaaggtcaagcgctggaaggtcggcgacgaggtcgtgatccactgcaatcaggatgatggtgacgacgagcactgcaatggcggtgacccgatgtattcgcccagtcagcggatctggggttacgagacgccggacggatcctttgctcagttcaccaatgtgcaggcgcagcagctgatgccgcggcccaagcacctgacctgggaagaagcggcatgttacacgctgacgctggcgaccgcctaccggatgctgtttggccatgagccgcatgatctcaagcccggtcagaacgttctggtctggggtgcgtccggtggtctggggtcctatgcgatccagcttatcaatacggcgggtgcgaacgcgattggcgtcatctcggatgaaagcaagcgccagtttgtcatggaccttggcgcaaagggtgtcatcaaccgcaaggatttcaactgctggggtcaactgcccacggtgaacacccccgaatatgccgagtggttcaaggaagcccgcaagttcggcaaggcgatctgggacattaccggcaagggcgtgaacgtggacatggtcttcgagcaccccggcgagagcacgttcccggtctcgaccttcgtggtgaagaagggcggtatggttgtgatctgcgcgggcaccagcggctacaacctgacctttgacgtgcgctatatgtggatgcaccagaagcgccttcagggcagccacttcgcccatctcaagcaggcaatggccgcgaaccagctgatggtcgagcgccggctcgacccgtgcatgtccgaggtgttcacctgggccgatctgcccgaggcgcatatgaagatgatgcgcaacgagcacaagccgggcaacatgtcggtgctggtgcaatcgccccgcaccgggctgcgcaccctcgaagaggttctggacgcccgcggttaa

SEQ ID NO: 14 - protein, 428 aa, Silicibacter pomeroyi
MALDTDSGIASYAAPEKDLYEMGEIPPMGFVPKKMYAWAIRKERHGDPDTAMQVEVVDVPTLDSHEVLVLVMAAGVNYNGVWASKGVPISPFDGHGQPYHIAGSDASGIVWAVGDKVKRWKVGDEVVIHCNQDDGDDEHCNGGDPMYSPSQRIWGYETPDGSFAQFTNVQAQQLMPRPKHLTWEEAACYTLTLATAYRMLFGHEPHDLKPGQNVLVWGASGGLGSYAIQLINTAGANAIGVISDESKRQFVMDLGAKGVINRKDFNCWGQLPTVNTPEYAEWFKEARKFGKAIWDITGKGVNVDMVFEHPGESTFPVSTFVVKKGGMVVICAGTSGYNLTFDVRYMWMHQKRLQGSHFAHLKQAMAANQLMVERRLDPCMSEVFTWADLPEAHMKMMRNEHKPGNMSVLVQSPRTGLRTLEEVLDARG

SEQ ID NO: 15 - DNA, 1284 bp, Xanthobacter autotrophicus
  CDS (1)..(1284)
atggcccagacggcagccgccaacgcgaacgagggaccggtgaaggacctttatgagctgggcgaggttccccccctcggtcacgtccccgccaagatgtacgcctgggccatccgccgcgagcgccatgggccgccggaagagtcgttccagctggaagtggtgcccacctgggagctgggcgagaacgacgtgctggtctacgtcatggccgccggcgtcaactacaacggcatctgggcgggcctcggccagccgatctcgccgttcgacgtgcacaaggcgcccttccacatcgccggctccgatgcctcgggtatcgtctgggcggtgggctccaaggtgaagcgctggaaggtgggcgacgaggtggtcgtgcactgtaaccaggacgacggcgacgacgaggagtgcaacggcggcgaccccatgttctccccgtcccagcgcatctggggctatgagacgccggacggctcgttcgcccagttctgccgggtgcaggcgcgccagctgatgccgcgccccaagcacctgacctgggaagagagcgcctgctacaccctcaccatggccaccgcctaccgcatgctgttcggccatccgccgcacacggtgaagccgggcgactacgtgctggtgtggggcgcctcgggcggcctcggcgtgttcggcgtgcagctcgccgccgcctccggcgcccatgtgatcggcgtgatctccgacgagaccaagcgcgactatgtcctcggcctcggcgccaagggcgtgatcaaccgcaaggatttcaagtgctggggccagctgcccaaggtcaactcgccggaatacaatgagtggaccaaggaagcccgcaagttcggcaaggccatttgggacatcagcggcaagcgcgacgtggacatcgtgttcgagcatcctggcgagcagaccttcccggtctcgaccctcgtgggcaagcgcggcggcatgatcgtgttctgcgccggcaccaccggcttcaacatcaccttcgacgcccgctacgtgtggatgcgccagaagcgcatccagggctcccacttcgctcacctcaagcaggcctccgccgccaatcagttcatcatcgaccggcgcgtggacccctgcatgtcggaagtgtttccgtgggaccgcatccccgaggcgcacaccaagatgtggaagaaccagcacgcccctggcaacatggcggtgctggtcaacaccccccgcaccggcctgcgtaccctcgaggacgtgatcgaggccggcgcgaagaagtga

SEQ ID NO: 16 - protein, 427 aa, Xanthobacter autotrophicus
MAQTAAANANEGPVKDLYELGEVPPLGHVPAKMYAWAIRRERHGPPEESFQLEVVPTWELGENDVLVYVMAAGVNYNGIWAGLGQPISPFDVHKAPFHIAGSDASGIVWAVGSKVKRWKVGDEVVVHCNQDDGDDEECNGGDPMFSPSQRIWGYETPDGSFAQFCRVQARQLMPRPKHLTWEESACYTLTMATAYRMLFGHPPHTVKPGDYVLVWGASGGLGVFGVQLAAASGAHVIGVISDETKRDYVLGLGAKGVINRKDFKCWGQLPKVNSPEYNEWTKEARKFGKAIWDISGKRDVDIVFEHPGEQTFPVSTLVGKRGGMIVFCAGTTGFNITFDARYVWMRQKRIQGSHFAHLKQASAANQFIIDRRVDPCMSEVFPWDRIPEAHTKMWKNQHAPGNMAVLVNTPRTGLRTLEDVIEAGAKK

SEQ ID NO: 17 - DNA, 2137 bp, Streptomyces sp. CL190
  CDS (647)..(1636)
cctgcaggccgtcgagggcgcctggaaggactacgcggagcaggacggccggtcgctggaggagttcgcggcgttcgtctaccaccagccgttcacgaagatggcctacaaggcgcaccgccacctgctgaacttcaacggctacgacaccgacaaggacgccatcgagggcgccctcggccagacgacggcgtacaacaacgtcatcggcaacagctacaccgcgtcggtgtacctgggcctggccgccctgctcgaccaggcggacgacctgacgggccgttccatcggcttcctgagctacggctcgggcagcgtcgccgagttcttctcgggcaccgtcgtcgccgggtaccgcgagcgtctgcgcaccgaggcgaaccaggaggcgatcgcccggcgcaagagcgtcgactacgccacctaccgcgagctgcacgagtacacgctcccgtccgacggcggcgaccacgccaccccggtgcagaccaccggccccttccggctggccgggatcaacgaccacaagcgcatctacgaggcgcgctagcgacacccctcggcaacggggtgcgccactgttcggcgcaccccgtgccgggctttcgcacagctattcacgaccatttgaggggcgggcagccgcatgaccgacgtccgattccgcattatcggtacgggtgcctacgtaccggaacggatcgtctccaacgatgaagtcggcgcgccggccggggtggacgacgactggatcacccgcaagaccggtatccggcagcgtcgctgggccgccgacgaccaggccacctcggacctggccacggccgcggggcgggcagcgctgaaagcggcgggcatcacgcccgagcagctgaccgtgatcgcggtcgccacctccacgccggaccggccgcagccgcccacggcggcctatgtccagcaccacctcggtgcgaccggcactgcggcgttcgacgtcaacgcggtctgctccggcaccgtgttcgcgctgtcctcggtggcgggcaccctcgtgtaccggggcggttacgcgctggtcatcggcgcggacctgtactcgcgcatcctcaacccggccgaccgcaagacggtcgtgctgttcggggacggcgccggcgcaatggtcctcgggccgacctcgaccggcacgggccccatcgtccggcgcgtcgccctgcacaccttcggcggcctcaccgacctgatccgtgtgcccgcgggcggcagccgccagccgctggacacggatggcctcgacgcgggactgcagtacttcgcgatggacgggcgtgaggtgcgccgcttcgtcacggagcacctgccgcagctgatcaagggcttcctgcacgaggccggggtcgacgccgccgacatcagccacttcgtgccgcatcaggccaacggtgtcatgctcgacgaggtcttcggcgagctgcatctgccgcgggcgaccatgcaccggacggtcgagacctacggcaacacgggagcggcctccatcccgatcaccatggacgcggccgtgcgcgccggttccttccggccgggcgagctggtcctgctggccgggttcggcggcggcatggccgcgagcttcgccctgatcgagtggtagtcgcccgtaccaccacagcggtccggcgccacctgttccctgcgccgggccgccctcggggcctttaggccccacaccgccccagccgacggattcagtcgcggcagtacctcagatgtccgctgcgacggcgtcccggagagcccgggcgagatcgcgggcccccttctgctcgtccccggcccctcccgcgagcaccacccgcggcggacggccgccgtcctccgcgatacgccgggcgaggtcgcaggcgagcacgccggacccggagaagccccccagcaccagcgaccggccgactccgtgcgcggccagggcaggctgcgcgccgtcgacgtcggtgagcagcaccaggagctcctgcggcccggcgtagaggtcggccagccggtcgtagcaggtcgcgggcgcgcccggcggcgggatcagacagatcgtgcccgcccgctcgtgcctcgccgcccgcagcgtgaccagcggaatgtcccgcccagctccgga

SEQ ID NO: 18 - protein, 329 aa, Streptomyces sp. CL190
MTDVRFRIIGTGAYVPERIVSNDEVGAPAGVDDDWITRKTGIRQRRWAADDQATSDLATAAGRAALKAAGITPEQLTVIAVATSTPDRPQPPTAAYVQHHLGATGTAAFDVNAVCSGTVFALSSVAGTLVYRGGYALVIGADLYSRILNPADRKTVVLFGDGAGAMVLGPTSTGTGPIVRRVALHTFGGLTDLIRVPAGGSRQPLDTDGLDAGLQYFAMDGREVRRFVTEHLPQLIKGFLHEAGVDAADISHFVPHQANGVMLDEVFGELHLPRATMHRTVETYGNTGAASIPITMDAAVRAGSFRPGELVLLAGFGGGMAASFALIEW

SEQ ID NO: 19 - DNA, 849 bp, Clostridium acetobutylicum
  CDS (1)..(849)
atgaaaaaggtatgtgttataggtgcaggtactatgggttcaggaattgctcaggcatttgcagctaaaggatttgaagtagtattaagagatattaaagatgaatttgttgatagaggattagattttatcaataaaaatctttctaaattagttaaaaaaggaaagatagaagaagctactaaagttgaaatcttaactagaatttccggaacagttgaccttaatatggcagctgattgcgatttagttatagaagcagctgttgaaagaatggatattaaaaagcagatttttgctgacttagacaatatatgcaagccagaaacaattcttgcatcaaatacatcatcactttcaataacagaagtggcatcagcaactaaaagacctgataaggttataggtatgcatttctttaatccagctcctgttatgaagcttgtagaggtaataagaggaatagctacatcacaagaaacttttgatgcagttaaagagacatctatagcaataggaaaagatcctgtagaagtagcagaagcaccaggatttgttgtaaatagaatattaataccaatgattaatgaagcagttggtatattagcagaaggaatagcttcagtagaagacatagataaagctatgaaacttggagctaatcacccaatgggaccattagaattaggtgattttataggtcttgatatatgtcttgctataatggatgttttatactcagaaactggagattctaagtatagaccacatacattacttaagaagtatgtaagagcaggatggcttggaagaaaatcaggaaaaggtttctacgattattcaaaataa

SEQ ID NO: 20 - protein, 282 aa, Clostridium acetobutylicum
MKKVCVIGAGTMGSGIAQAFAAKGFEVVLRDIKDEFVDRGLDFINKNLSKLVKKGKIEEATKVEILTRISGTVDLNMAADCDLVIEAAVERMDIKKQIFADLDNICKPETILASNTSSLSITEVASATKRPDKVIGMHFFNPAPVMKLVEVIRGIATSQETFDAVKETSIAIGKDPVEVAEAPGFVVNRILIPMINEAVGILAEGIASVEDIDKAMKLGANHPMGPLELGDFIGLDICLAIMDVLYSETGDSKYRPHTLLKKYVRAGWLGRKSGKGFYDYSK

SEQ ID NO: 21 - DNA, 786 bp, Clostridium acetobutylicum
  CDS (1)..(786)
atggaactaaacaatgtcatccttgaaaaggaaggtaaagttgctgtagttaccattaacagacctaaagcattaaatgcgttaaatagtgatacactaaaagaaatggattatgttataggtgaaattgaaaatgatagcgaagtacttgcagtaattttaactggagcaggagaaaaatcatttgtagcaggagcagatatttctgagatgaaggaaatgaataccattgaaggtagaaaattcgggatacttggaaataaagtgtttagaagattagaacttcttgaaaagcctgtaatagcagctgttaatggttttgctttaggaggcggatgcgaaatagctatgtcttgtgatataagaatagcttcaagcaacgcaagatttggtcaaccagaagtaggtctcggaataacacctggttttggtggtacacaaagactttcaagattagttggaatgggcatggcaaagcagcttatatttactgcacaaaatataaaggcagatgaagcattaagaatcggacttgtaaataaggtagtagaacctagtgaattaatgaatacagcaaaagaaattgcaaacaaaattgtgagcaatgctccagtagctgttaagttaagcaaacaggctattaatagaggaatgcagtgtgatattgatactgctttagcatttgaatcagaagcatttggagaatgcttttcaacagaggatcaaaaggatgcaatgacagctttcatagagaaaagaaaaattgaaggcttcaaaaatagatag

SEQ ID NO: 22 - protein, 261 aa, Clostridium acetobutylicum
MELNNVILEKEGKVAVVTINRPKALNALNSDTLKEMDYVIGEIENDSEVLAVILTGAGEKSFVAGADISEMKEMNTIEGRKFGILGNKVFRRLELLEKPVIAAVNGFALGGGCEIAMSCDIRIASSNARFGQPEVGLGITPGFGGTQRLSRLVGMGMAKQLIFTAQNIKADEALRIGLVNKVVEPSELMNTAKEIANKIVSNAPVAVKLSKQAINRGMQCDIDTALAFESEAFGECFSTEDQKDAMTAFIEKRKIEGFKNR

SEQ ID NO: 23 - protein, 398 aa, Treponema denticola
  misc_feature (398)..(398): Xaa can be any naturally occurring amino acid
MIVKPMVRNNICLNAHPQGCKKGVEDQIEYTKKRITAEVKAGAKAPKNVLVLGCSNGYGLASRITAAFGYGAATIGVSFEKAGSETKYGTPGWYNNLAFDEAAKREGLYSVTIDGDAFSDEIKAQVIEEAKKKGIKFDLIVYSLASPVRTDPDTGIMHKSVLKPFGKTFTGKTVDPFTGELKEISAEPANDEEAAATVKVMGGEDWERWIKQLSKEGLLEEGCITLAYSYIGPEATQALYRKGTIGKAKEHLEATAHRLNKENPSIRAFVSVNKGLVTRASAVIPVIPLYLASLFKVMKEKGNHEGCIEQITRLYAERLYRKDGTIPVDEENRIRIDDWELEEDVQKAVSALMEKVTGENAESLTDLAGYRHDFLASNGFDVEGINYEAEVERFDRIX

SEQ ID NO: 24 - protein, 399 aa, Treponema vincentii
  misc_feature (399)..(399): Xaa can be any naturally occurring amino acid
MSMKPMLRSNICLNAHPQGCKKAVEDQIAYTRKRAASHPAGTATPKNVLVIGCSGGYGLASRITAAFGYGAATIGVSYEKAGSEKKWGTPGWYNNLAVDAAAKEAGLISVTINGDAFSDAIKAQVIDEAKKLNIKFDLIVYSVASSVRTDPDTGVTYRSALKPFGKPFTGKTLDPFTGALTEITAEPATDEEAAATVKVMGGEDWQRWIEKLGAADVLAQGCITVAYSYIGPEATQALYRKGTIGKAKEHLEATAHALNAKLAALKGKAFVSVNKGLVTRASAVIPVIPLYLASLFKVMKEKGTHEGCIEQINRLFDSRLYTADGVIPTDNENRIRIDDWELDEGVQSAVAKIMATVTDETSCKLTDVDEYRHDFLAINGFDIAGIDYDAEIDRFDRIX

SEQ ID NO: 25 - protein, 406 aa, Fibrobacter succinogenes
  misc_feature (406)..(406): Xaa can be any naturally occurring amino acid
MIIKPLIRSNMCINAHPKGCAADVKHQIEFIKKKFTTRSIPADAPKTVLVLGCSTGYGLASRIVAAFGYKAATIGVSFEKEGSDGGIGESREKTGTPGWYNNMAFDKFAKEAGLDAVTFNGDAFSHEMRQNVIDTLKKMGRKVDLLVYSVASSVRVDPDNGTIYRSVLKPIDKVFTGATIDCLSGKISTISAEPATAEEAANTVKVMGGEDWALWVRKLKEAGVLAEGVKTVAYSYIGPKLSHAIYRDGTIGGAKKHLEATALELNKELQNDLHGEAYVSVNKGLVTRSSAVIPIIPMYISVLFKVMKEMGNHEGCIEQMERLMTERLYTGSKVPTDENHLIRIDDYELDPKVQAEVDKRMATVTQENLAEVGDLEGYRHDFLATNGFDIDGVDYEADVQTLTSIX

SEQ ID NO: 26 - protein, 397 aa, Flavobacterium johnsoniae
  misc_feature (397)..(397): Xaa can be any naturally occurring amino acid
MIIEPRMRGFICLTAHPAGCEQNVKNQIEYIKSKGAIAGAKKVLVIGASTGFGLASRITSAFGSDAATIGVFFEKPPVEGKTASPGWYNSAAFEKEAHKAGLYAKSINGDAFSNEIKRETLDLIKADLGQVDLVIYSLASPVRTNPNTGVTHRSVLKPIGQTFTNKTVDFHTGNVSEVSIAPANEEDIENTVAVMGGEDWAMWIDALKNENLLAEGATTIAYSYIGPELTEAVYRKGTIGRAKDHLEATAFTITDTLKSLGGKAYVSVNKALVTQASSAIPVIPLYISLLYKIMKEEGIHEGCIEQIQRLFQDRLYNGSEVPVDEKGRIRIDDWEMREDVQAKVAALWKEATTETLPSIGDLAGYRNDFLNLFGFEFAGVDYKADTNEVVNIESIKX

SEQ ID NO: 27 - DNA, 405 bp, Aeromonas caviae
  CDS (1)..(405)
atgagcgcacaatccctggaagtaggccagaaggcccgtctcagcaagcggttcggggcggcggaggtagccgccttcgccgcgctctcggaggacttcaaccccctgcacctggacccggccttcgccgccaccacggcgttcgagcggcccatagtccacggcatgctgctcgccagcctcttctccgggctgctgggccagcagttgccgggcaaggggagcatctatctgggtcaaagcctcagcttcaagctgccggtctttgtcggggacgaggtgacggccgaggtggaggtgaccgcccttcgcgaggacaagcccatcgccaccctgaccacccgcatcttcacccaaggcggcgccctcgccgtgacgggggaagccgtggtcaagctgccttaa

SEQ ID NO: 28 - protein, 134 aa, Aeromonas caviae
MSAQSLEVGQKARLSKRFGAAEVAAFAALSEDFNPLHLDPAFAATTAFERPIVHGMLLASLFSGLLGQQLPGKGSIYLGQSLSFKLPVFVGDEVTAEVEVTALREDKPIATLTTRIFTQGGALAVTGEAVVKLP

SEQ ID NO: 29 - DNA, 711 bp, Ralstonia eutropha H16
  CDS (1)..(711)
atgggtgggctgggagaagccatcagcatcaagctgcacgacgcaggctatgcggtggtggtgacgcactcgccgggcaatgcggccgcccaggactggcttgccgcgatggccgccggcggccgcgagatacgtgcctacgaggtggatgtatccgactacgatgcctgccaggcttgcgcggcgcagatcctggctgacgtaggccgcgtggatatcctggtgaacaacgccggcattacccgcgacatggccttcaagaagatggacaagccgaactgggacgccgtgatgcggaccaatctcgactcggtgttcaatctcaccaagccgctttgcgaaggcatggtcgaacgcggctggggacgcatcatcaacatctcatcggtcaacgcatccaagggcgccttcggccagaccaactatgccgccgccaaggccgggatgcacggcttcaccaagtcgctggcgctggaagtggcaaggaagggcgtgaccgtcaacaccgtctctccgggctaccttgccaccaagatggtcaacgccgtgcccaaggaaatcatggagaccaagatcctgccgcagattccggtcggccgcgtcggcaagccggaagaagtcgcagcgctgattgcctacctgtgctcggaggaagcggcctacgtgaccgggtccaatatcgccatcaatggcgggcagcacatgcagtaa

SEQ ID NO: 30 - protein, 236 aa, Ralstonia eutropha H16
MGGLGEAISIKLHDAGYAVVVTHSPGNAAAQDWLAAMAAGGREIRAYEVDVSDYDACQACAAQILADVGRVDILVNNAGITRDMAFKKMDKPNWDAVMRTNLDSVFNLTKPLCEGMVERGWGRIINISSVNASKGAFGQTNYAAAKAGMHGFTKSLALEVARKGVTVNTVSPGYLATKMVNAVPKEIMETKILPQIPVGRVGKPEEVAALIAYLCSEEAAYVTGSNIAINGGQHMQ

SEQ ID NO: 31 - DNA, 1164 bp, Escherichia coli
  CDS (1)..(1164)
atgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtgctggaatttggcggtattgagccaaacccggcttatgaaacgctgatgaacgccgtgaaactggttcgcgaacagaaagtgactttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagccggtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctaatcgaagatggtccgaaagccctgaaagagccagaaaactacgatgtgcgcgccaacgtcatgtgggcggcgactcaggcgctgaacggtttgattggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttccgatgatgagcgtattgacgccgcgattgccgcaacccgcaatttctttgagcaattaggcgtgccgacccacctctccgactacggtctggacggcagctccatcccggctttgctgaaaaaactggaagagcacggcatgacccaactgggcgaaaatcatgacattacgttggatgtcagccgccgtatatacgaagccgcccgctaa

SEQ ID NO: 32 - protein, 387 aa, Escherichia coli
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLDGTKFIAAAANYPENIDPWHILQTGGKEIKSAIPMGCVLTLPATGSESNAGAVISRKTTGDKQAFHSAHVQPVFAVLDPVYTYTLPPRQVANGVVDAFVHTVEQYVTKPVDAKIQDRFAEGILLTLIEDGPKALKEPENYDVRANVMWAATQALNGLIGAGVPQDWATHMLGHELTAMHGLDHAQTLAIVLPALWNEKRDTKRAKLLQYAERVWNITEGSDDERIDAAIAATRNFFEQLGVPTHLSDYGLDGSSIPALLKKLEEHGMTQLGENHDITLDVSRRIYEAAR

SEQ ID NO: 33 - DNA, 1395 bp, Salmonella enterica
  CDS (1)..(1395)
atgaatacttctgaactcgaaaccctgattcgcaccattcttagcgagcaattaaccacgccggcgcaaacgccggtccagcctcagggcaaagggattttccagtccgtgagcgaggccatcgacgccgcgcaccaggcgttcttacgttatcagcagtgcccgctaaaaacccgcagcgccattatcagcgcgatgcgtcaggagctgacgccgctgctggcgcccctggcggaagagagcgccaatgaaacggggatgggcaacaaagaagataaatttctcaaaaacaaggctgcgctggacaacacgccgggcgtagaagatctcaccaccaccgcgctgaccggcgacggcggcatggtgctgtttgaatactcaccgtttggcgttatcggttcggtcgccccaagcaccaacccgacggaaaccatcatcaacaacagtatcagcatgctggcggcgggcaacagtatctactttagcccgcatccgggagcgaaaaaggtctctctgaagctgattagcctgattgaagagattgccttccgctgctgcggcatccgcaatctggtggtgaccgtggcggaacccaccttcgaagcgacccagcagatgatggcccacccgcgaatcgcagtactggccattaccggcggcccgggcattgtggcaatgggcatgaagagcggtaagaaggtgattggcgctggcgcgggtaacccgccctgcatcgttgatgaaacggcggacctggtgaaagcggcggaagatatcatcaacggcgcgtcattcgattacaacctgccctgcattgccgagaagagcctgatcgtagtggagagtgtcgccgaacgtctggtgcagcaaatgcaaaccttcggcgcgctgctgttaagccctgccgataccgacaaactccgcgccgtctgcctgcctgaaggccaggcgaataaaaaactggtcggcaagagcccatcggccatgctggaagccgccgggatcgctgtccctgcaaaagcgccgcgtctgctgattgcgctggttaacgctgacgatccgtgggtcaccagcgaacagttgatgccgatgctgccagtggtaaaagtcagcgatttcgatagcgcgctggcgctggccctgaaggttgaagaggggctgcatcataccgccattatgcactcgcagaacgtgtcacgcctgaacctcgcggcccgcacgctgcaaacctcgatattcgtcaaaaacggcccctcttatgccgggatcggcgtcggcggcgaaggctttaccaccttcactatcgccacaccaaccggtgaagggaccacgtcagcgcgtacttttgcccgttcccggcgctgcgtactgaccaacggcttttctattcgctaa

SEQ ID NO: 34 - protein, 464 aa, Salmonella enterica
MNTSELETLIRTILSEQLTTPAQTPVQPQGKGIFQSVSEAIDAAHQAFLRYQQCPLKTRSAIISAMRQELTPLLAPLAEESANETGMGNKEDKFLKNKAALDNTPGVEDLTTTALTGDGGMVLFEYSPFGVIGSVAPSTNPTETIINNSISMLAAGNSIYFSPHPGAKKVSLKLISLIEEIAFRCCGIRNLVVTVAEPTFEATQQMMAHPRIAVLAITGGPGIVAMGMKSGKKVIGAGAGNPPCIVDETADLVKAAEDIINGASFDYNLPCIAEKSLIVVESVAERLVQQMQTFGALLLSPADTDKLRAVCLPEGQANKKLVGKSPSAMLEAAGIAVPAKAPRLLIALVNADDPWVTSEQLMPMLPVVKVSDFDSALALALKVEEGLHHTAIMHSQNVSRLNLAARTLQTSIFVKNGPSYAGIGVGGEGFTTFTIATPTGEGTTSARTFARSRRCVLTNGFSIR

SEQ ID NO: 35 - DNA, 960 bp, Escherichia coli
  CDS (1)..(960)
atgagtctgaatttccttgattttgaacagccgattgcagagctggaagcgaaaatcgattctctgactgcggttagccgtcaggatgagaaactggatattaacatcgatgaagaagtgcatcgtctgcgtgaaaaaagcgtagaactgacacgtaaaatcttcgccgatctcggtgcatggcagattgcgcaactggcacgccatccacagcgtccttataccctggattacgttcgcctggcatttgatgaatttgacgaactggctggcgaccgcgcgtatgcagacgataaagctatcgtcggtggtatcgcccgtctcgatggtcgtccggtgatgatcattggtcatcaaaaaggtcgtgaaaccaaagaaaaaattcgccgtaactttggtatgccagcgccagaaggttaccgcaaagcactgcgtctgatgcaaatggctgaacgctttaagatgcctatcatcacctttatcgacaccccgggggcttatcctggcgtgggcgcagaagagcgtggtcagtctgaagccattgcacgcaacctgcgtgaaatgtctcgcctcggcgtaccggtagtttgtacggttatcggtgaaggtggttctggcggtgcgctggcgattggcgtgggcgataaagtgaatatgctgcaatacagcacctattccgttatctcgccggaaggttgtgcgtccattctgtggaagagcgccgacaaagcgccgctggcggctgaagcgatgggtatcattgctccgcgtctgaaagaactgaaactgatcgactccatcatcccggaaccactgggtggtgctcaccgtaacccggaagcgatggcggcatcgttgaaagcgcaactgctggcggatctggccgatctcgacgtgttaagcactgaagatttaaaaaatcgtcgttatcagcgcctgatgagctacggttacgcgtaa

SEQ ID NO: 36 - protein, 319 aa, Escherichia coli
MSLNFLDFEQPIAELEAKIDSLTAVSRQDEKLDINIDEEVHRLREKSVELTRKIFADLGAWQIAQLARHPQRPYTLDYVRLAFDEFDELAGDRAYADDKAIVGGIARLDGRPVMIIGHQKGRETKEKIRRNFGMPAPEGYRKALRLMQMAERFKMPIITFIDTPGAYPGVGAEERGQSEAIARNLREMSRLGVPVVCTVIGEGGSGGALAIGVGDKVNMLQYSTYSVISPEGCASILWKSADKAPLAAEAMGIIAPRLKELKLIDSIIPEPLGGAHRNPEAMAASLKAQLLADLADLDVLSTEDLKNRRYQRLMSYGYA

SEQ ID NO: 37 - DNA, 471 bp, Escherichia coli
  CDS (1)..(471)
atggatattcgtaagattaaaaaactgatcgagctggttgaagaatcaggcatctccgaactggaaatttctgaaggcgaagagtcagtacgcattagccgtgcagctcctgccgcaagtttccctgtgatgcaacaagcttacgctgcaccaatgatgcagcagccagctcaatctaacgcagccgctccggcgaccgttccttccatggaagcgccagcagcagcggaaatcagtggtcacatcgtacgttccccgatggttggtactttctaccgcaccccaagcccggacgcaaaagcgttcatcgaagtgggtcagaaagtcaacgtgggcgataccctgtgcatcgttgaagccatgaaaatgatgaaccagatcgaagcggacaaatccggtaccgtgaaagcaattctggtcgaaagtggacaaccggtagaatttgacgagccgctggtcgtcatcgagtaa

SEQ ID NO: 38 - protein, 156 aa, Escherichia coli
MDIRKIKKLIELVEESGISELEISEGEESVRISRAAPAASFPVMQQAYAAPMMQQPAQSNAAAPATVPSMEAPAAAEISGHIVRSPMVGTFYRTPSPDAKAFIEVGQKVNVGDTLCIVEAMKMMNQIEADKSGTVKAILVESGQPVEFDEPLVVIE

SEQ ID NO: 39 - DNA, 1350 bp, Escherichia coli
  CDS (1)..(1350)
atgctggataaaattgttattgccaaccgcggcgagattgcattgcgtattcttcgtgcctgtaaagaactgggcatcaagactgtcgctgtgcactccagcgcggatcgcgatctaaaacacgtattactggcagatgaaacggtctgtattggccctgctccgtcagtaaaaagttatctgaacatcccggcaatcatcagcgccgctgaaatcaccggcgcagtagcaatccatccgggttacggcttcctctccgagaacgccaactttgccgagcaggttgaacgctccggctttatcttcattggcccgaaagcagaaaccattcgcctgatgggcgacaaagtatccgcaatcgcggcgatgaaaaaagcgggcgtcccttgcgtaccgggttctgacggcccgctgggcgacgatatggataaaaaccgtgccattgctaaacgcattggttatccggtgattatcaaagcctccggcggcggcggcggtcgcggtatgcgcgtagtgcgcggcgacgctgaactggcacaatccatctccatgacccgtgcggaagcgaaagctgctttcagcaacgatatggtttacatggagaaatacctggaaaatcctcgccacgtcgagattcaggtactggctgacggtcagggcaacgctatctatctggcggaacgtgactgctccatgcaacgccgccaccagaaagtggtcgaagaagcgccagcaccgggcattaccccggaactgcgtcgctacatcggcgaacgttgcgctaaagcgtgtgttgatatcggctatcgcggtgcaggtactttcgagttcctgttcgaaaacggcgagttctatttcatcgaaatgaacacccgtattcaggtagaacacccggttacagaaatgatcaccggcgttgacctgatcaaagaacagctgcgtatcgctgccggtcaaccgctgtcgatcaagcaagaagaagttcacgttcgcggccatgcggtggaatgtcgtatcaacgccgaagatccgaacaccttcctgccaagtccgggcaaaatcacccgtttccacgcacctggcggttttggcgtacgttgggagtctcatatctacgcgggctacaccgtaccgccgtactatgactcaatgatcggtaagctgatttgctacggtgaaaaccgtgacgtggcgattgcccgcatgaagaatgcgctgcaggagctgatcatcgacggtatcaaaaccaacgttgatctgcagatccgcatcatgaatgacgagaacttccagcatggtggcactaacatccactatctggagaaaaaactcggtcttcaggaaaaataa

SEQ ID NO: 40 - protein, 449 aa, Escherichia coli
MLDKIVIANRGEIALRILRACKELGIKTVAVHSSADRDLKHVLLADETVCIGPAPSVKSYLNIPAIISAAEITGAVAIHPGYGFLSENANFAEQVERSGFIFIGPKAETIRLMGDKVSAIAAMKKAGVPCVPGSDGPLGDDMDKNRAIAKRIGYPVIIKASGGGGGRGMRVVRGDAELAQSISMTRAEAKAAFSNDMVYMEKYLENPRHVEIQVLADGQGNAIYLAERDCSMQRRHQKVVEEAPAPGITPELRRYIGERCAKACVDIGYRGAGTFEFLFENGEFYFIEMNTRIQVEHPVTEMITGVDLIKEQLRIAAGQPLSIKQEEVHVRGHAVECRINAEDPNTFLPSPGKITRFHAPGGFGVRWESHIYAGYTVPPYYDSMIGKLICYGENRDVAIARMKNALQELIIDGIKTNVDLQIRIMNDENFQHGGTNIHYLEKKLGLQEK

SEQ ID NO: 41 - DNA, 915 bp, Escherichia coli
  CDS (1)..(915)
atgagctggattgaacgaattaaaagcaacattactcccacccgcaaggcgagcattcctgaaggggtgtggactaagtgtgatagctgcggtcaggttttataccgcgctgagctggaacgtaatcttgaggtctgtccgaagtgtgaccatcacatgcgtatgacagcgcgtaatcgcctgcatagcctgttagatgaaggaagccttgtggagctgggtagcgagcttgagccgaaagatgtgctgaagtttcgtgactccaagaagtataaagaccgtctggcatctgcgcagaaagaaaccggcgaaaaagatgcgctggtggtgatgaaaggcactctgtatggaatgccggttgtcgctgcggcattcgagttcgcctttatgggcggttcaatggggtctgttgtgggtgcacgtttcgtgcgtgccgttgagcaggcgctggaagataactgcccgctgatctgcttctccgcctctggtggcgcacgtatgcaggaagcactgatgtcgctgatgcagatggcgaaaacctctgcggcactggcaaaaatgcaggagcgcggcttgccgtacatctccgtgctgaccgacccgacgatgggcggtgtttctgcaagtttcgccatgctgggcgatctcaacatcgctgaaccgaaagcgttaatcggctttgccggtccgcgtgttatcgaacagaccgttcgcgaaaaactgccgcctggattccagcgcagtgaattcctgatcgagaaaggcgcgatcgacatgatcgtccgtcgtccggaaatgcgcctgaaactggcgagcattctggcgaagttgatgaatctgccagcgccgaatcctgaagcgccgcgtgaaggcgtagtggtacccccggtaccggatcaggaacctgaggcctga

SEQ ID NO: 42 - protein, 304 aa, Escherichia coli
MSWIERIKSNITPTRKASIPEGVWTKCDSCGQVLYRAELERNLEVCPKCDHHMRMTARNRLHSLLDEGSLVELGSELEPKDVLKFRDSKKYKDRLASAQKETGEKDALVVMKGTLYGMPVVAAAFEFAFMGGSMGSVVGARFVRAVEQALEDNCPLICFSASGGARMQEALMSLMQMAKTSAALAKMQERGLPYISVLTDPTMGGVSASFAMLGDLNIAEPKALIGFAGPRVIEQTVREKLPPGFQRSEFLIEKGAIDMIVRRPEMRLKLASILAKLMNLPAPNPEAPREGVVVPPVPDQEPEA

SEQ ID NO: 43 - DNA, 1566 bp, Aeromonas hydrophila
  CDS (1)..(1566)
atgctctcccgccagaacgcgagagaactggtgcgcaacgccaaacaggcgcaggtgattatggccaccttttcgcagcagaaaatcgacgccatcgtgaagaacgtggccgaagaagccgcgcgccatgccgaaacgctggcgaaaatggccgcagaagagaccggttttggcaactggcaggacaaggtgctgaaaaaccggttcgcttcgctgcacgtttacgatgccatcaaagagatgaagaccgtcgggatcattcatgacgatcaggcgaaaaaagtgatggatgtgggcgtgccgctgggcgtgatctgcgcgctggtaccctccaccaaccccacctccaccatcttctacaaaaccctgatcgccctcaaggcgggcaatgccatcatcttctcgccgcacccgggcgcacgccagtgcagctggaaagccatcgaaatcgtcaagcgcgccgccgaagccgccggtgccccggcgggcatcgtggacggtgtcacccagctgaccctggaggccacctccgagctgatgcacagcaaggacgtatcgctgatcctggcaaccggtggcgaaggcatggtgcgcgcggcctatgcctccggcaccccgaccatcagtggcggcccgggcaacggcccggcctttatcgagcgcagcgccgacattcaccaggcggtgaaggacatcatcaccagcaagaccttcgacaacggcgtgatctgcgcctccgaacagtcgatcatcgtcgaacgctgcatctacgacgaggttcatcgcgagctggcggcccagggcgcctacttcatgaacgaagacgaagccgccaggatggctgccctgctgctgcgcccgaacggcaccatcaacccgaaagtggtgggcaagacggcgctccatctcagccagctggccggtttcagcgtcccccccagcacccgcgtgctggtggccgaacagaccactgtctcccacagcaacccctactcccgtgaaaagctctgcccggtgctcggcctctacgtggaagaggagtggcgcgccgcctgtcatcgggtggttgagctgctgaccaacgaagggctgggccacaccctggtgatccatacccgcaatcaggacgtgatccgccagttcagcctggaaaaaccggtcaaccggatcctgatcaacaccccggccgccctcggcggcatcggtgccaccaccaacctcacaccggcactgaccctcggctgcggcgccgtcggcggcggctccagctccgacaacgtcggcccgatgaacctgctcaacatccgcaaggtgggttacggcgtgcgcaccatcgaagagctgcgcgcccccatccagccggttgcagtgcagcccgccagtgctgcccccaccgcaccccagccctgcagcatcctcgacgatgcccgcttcagtgccccggcgccggcctgccacagtgccgacgaccgttttgccggcgccagcgccgaggtcggcggcgagatcagcgagcagaacgtggagcgggtcatccgtcaggtgcttgagcgccttggcaagtaa

SEQ ID NO: 44 - protein, 521 aa, Aeromonas hydrophila
MLSRQNARELVRNAKQAQVIMATFSQQKIDAIVKNVAEEAARHAETLAKMAAEETGFGNWQDKVLKNRFASLHVYDAIKEMKTVGIIHDDQAKKVMDVGVPLGVICALVPSTNPTSTIFYKTLIALKAGNAIIFSPHPGARQCSWKAIEIVKRAAEAAGAPAGIVDGVTQLTLEATSELMHSKDVSLILATGGEGMVRAAYASGTPTISGGPGNGPAFIERSADIHQAVKDIITSKTFDNGVICASEQSIIVERCIYDEVHRELAAQGAYFMNEDEAARMAALLLRPNGTINPKVVGKTALHLSQLAGFSVPPSTRVLVAEQTTVSHSNPYSREKLCPVLGLYVEEEWRAACHRVVELLTNEGLGHTLVIHTRNQDVIRQFSLEKPVNRILINTPAALGGIGATTNLTPALTLGCGAVGGGSSSDNVGPMNLLNIRKVGYGVRTIEELRAPIQPVAVQPASAAPTAPQPCSILDDARFSAPAPACHSADDRFAGASAEVGGEISEQNVERVIRQVLERLGK

SEQ ID NO: 45 - DNA, 1389 bp, Klebsiella pneumoniae
  CDS (1)..(1389)
atgaatacagcagaactggaaacccttatccgcaccatcctcagtgaaaagctcgcgccgacgccccctgcccctcagcaagagcagggcattttctgcgatgtcggcagcgccatcgacgccgctcatcaggcttttctccgctatcagcagtgtccgctaaaaacccgcagcgccattatcagcgccctgcgggagacgctggcccccgagctggcgacgctggcggaagagagcgccacggaaaccggcatgggcaacaaagaagataaatatctgaaaaataaagccgctcttgaaaacacgccgggcatagaggatctcactaccagcgccctcaccggcgatggcgggatggtgctgtttgagtactcgccgttcggggttattggcgccgtggcgcccagcaccaacccaacggaaaccattatcaacaacagtatcagcatgctggcggcgggtaacagcgtctatttcagcccccatcccggcgcgaaaaaggtctcgttgaagcttatcgccaggatcgaagagatcgcctaccgctgcagcgggatccgtaacctggtggtgaccgttgccgagccgacctttgaagccacccagcaaatgatgtcccacccgctgattgccgttctggctatcaccggcggccctggcattgtggcgatgggcatgaaaagcggtaaaaaagtgatcggcgctggcgccggcaatccgccgtgcatcgttgatgaaaccgccgatctcgtcaaagccgccgaagatatcatcagcggcgccgccttcgattacaacctgccctgtatcgccgaaaaaagcctgatcgtcgtcgcctccgtcgctgaccgcctgatccagcagatgcaggattttgacgcgctgctgttgagccgacaggaggccgataccctgcgtgccgtctgcctgcccgacggcgcggcgaataaaaaactggtcggtaaaagcccggctgcgctgctggcggcggcgggtctcgccgttccgcctcgcccccctcgcctgctgatagccgaggtggaggcgaacgccccctgggtgacctgcgagcagctgatgccggtgctgccgatcgtcagggtcgccgactttgacagcgccctggcgctggccctgcgcgttgaggagggtctgcaccacaccgccattatgcactcgcagaatgtctcgcggctcaatctggcggcacgcaccctgcagacctccatttttgtcaaaaatggcccgtcttacgcgggaatcggcgtcggcggcgaagggtttaccaccttcaccatcgccacgccaaccggagaaggcaccacctccgcgcggacgttcgcccgcctgcggcgctgcgtgttgaccaacggtttttccattcgctaa

SEQ ID NO: 46 - protein, 462 aa, Klebsiella pneumoniae
MNTAELETLIRTILSEKLAPTPPAPQQEQGIFCDVGSAIDAAHQAFLRYQQCPLKTRSAIISALRETLAPELATLAEESATETGMGNKEDKYLKNKAALENTPGIEDLTTSALTGDGGMVLFEYSPFGVIGAVAPSTNPTETIINNSISMLAAGNSVYFSPHPGAKKVSLKLIARIEEIAYRCSGIRNLVVTVAEPTFEATQQMMSHPLIAVLAITGGPGIVAMGMKSGKKVIGAGAGNPPCIVDETADLVKAAEDIISGAAFDYNLPCIAEKSLIVVASVADRLIQQMQDFDALLLSRQEADTLRAVCLPDGAANKKLVGKSPAALLAAAGLAVPPRPPRLLIAEVEANAPWVTCEQLMPVLPIVRVADFDSALALALRVEEGLHHTAIMHSQNVSRLNLAARTLQTSIFVKNGPSYAGIGVGGEGFTTFTIATPTGEGTTSARTFARLRRCVLTNGFSIR

SEQ ID NO: 47 - DNA, 1434 bp, Lactobacillus brevis
  CDS (1)..(1434)
atgaacacagaaaacattgaacaagccatccgtaaaattttgagtgaagaacttagcaatcctcagtcatcaacggccaccaatacgaccgttcccggcaaaaatgggatctttaagacggtcaatgaagccattgcggccacaaaagcggcgcaagaaaactacgccgaccaaccaatctcagttcggaacaaagtgattgatgcgatccgtgagggtttccggccatacattgaggatatggctaagcggattcatgacgaaactggcatgggaacggttagcgcgaaaattgccaaactcaataacgccctttataacacacccggtccagaaattctgcagccagaagccgaaaccggtgacggtggactggttatgtatgaatacgcgccatttggtgtcattggtgccgttggtcctagtaccaacccctctgaaacggtgattgccaatgccatcatgatgttggctggtgggaatacgttgttctttggtgcccatccaggtgctaagaacattacccgttggacgatcgaaaaattaaacgaattggtagctgatgcaactgggttacataacttagtcgtttcactggaaacgccttcaattgaatccgtgcaagaagttatgcaacatcctgacgttgccatgctgtcaatcactggagggcctgctgttgtccaccaagcgcttatcagtggtaagaaggcggttggtgccggtgctggtaacccaccggcaatggtggatgcaactgccaatattgctttagcagcccacaacattgttgattcagcagcctttgataataacattctctgcacggccgaaaaggaagttgtcgttgaagccgctgtcaaggatgaactcatcatgcggatgcaacaagaaggggccttcttggttaccgattctgccgatattgaaaaattagcgcaaatgaccattgggcctaagggcgcaccagatcggaagtttgttggtaaagatgccacttacattttggatcaagcaggaatctcttacaccgggacaccaacactgattattcttgaagcagctaaggatcatccgttagtaacgacagaaatgttgatgccaattttgccagtcgtttgttgccctgactttgatagcgttttagcaacggctacagaagttgaaggtgggttacaccacacggcttccattcattccgagaatttaccacacatcaataaggcagcgcaccggttgaatacgtcaatcttcgtggttaacggcccaacttattgtgggactggtgttgcaacgaatggtgcgcatagtggggcttcagccttaacgattgccacaccaacgggtgaaggaacggcaacgtctaagacttacacgcgccggcgccggcttaactcgccagaagggttctcattacggacttgggaggcttag

SEQ ID NO: 48 - protein, 477 aa, Lactobacillus brevis
MNTENIEQAIRKILSEELSNPQSSTATNTTVPGKNGIFKTVNEAIAATKAAQENYADQPISVRNKVIDAIREGFRPYIEDMAKRIHDETGMGTVSAKIAKLNNALYNTPGPEILQPEAETGDGGLVMYEYAPFGVIGAVGPSTNPSETVIANAIMMLAGGNTLFFGAHPGAKNITRWTIEKLNELVADATGLHNLVVSLETPSIESVQEVMQHPDVAMLSITGGPAVVHQALISGKKAVGAGAGNPPAMVDATANIALAAHNIVDSAAFDNNILCTAEKEVVVEAAVKDELIMRMQQEGAFLVTDSADIEKLAQMTIGPKGAPDRKFVGKDATYILDQAGISYTGTPTLIILEAAKDHPLVTTEMLMPILPVVCCPDFDSVLATATEVEGGLHHTASIHSENLPHINKAAHRLNTSIFVVNGPTYCGTGVATNGAHSGASALTIATPTGEGTATSKTYTRRRRLNSPEGFSLRTWEA

SEQ ID NO: 49 - DNA, 1410 bp, Listeria monocytogenes
  CDS (1)..(1410)
atggaatcattagaactcgaaaaactagtgaaaaaagttctcttagaaaaattagcagaacaaaaaggtataccagtaaaaacaatgaccaaaggcgcaaaaagtggggtttttgatacagttgacgaggccgttcaagcagcagttatagcacaaaatagctataaagaaaaatcattagaagaacgccgcaacgttgtgaaagcaattcgcgaagcactttatccagaaattgaatccattgcggcgcgagcagttgctgaaacaggtatgggaaatgtggcagataaaattttgaaaaacacgttagcgattgaaaaaacgcccggcgttgaagatttgtacacagaagtcgctactggtgacaatggaatgacactttacgaactttctccatatggcgtaattggggcagtagcaccgagcacgaacccgacggagacattaatctgcaatacaatcggcatgctagcagctgggaatgcagtattttatagcccgcatccaggtgcgaaaaatatttctctttggttgattgaaaagttgaatacgattgtccgcgaaagttgtggtgttgataatttagttgttacagttgaaaaaccatccattcaagccgcgcaagaaatgatgaatcatccgaaagtaccgttgctcgttattacaggtggccctggtgtagttcttcaagccatgcaatccggtaaaaaagttattggtgcaggtgctgggaatccgccatccattgtagatgagacagcgaacatcgaaaaagcagctgctgatatcgtagacggcgcatcttttgaccataatattctatgtattgcggagaaaagtgttgttgcagttgatagcatcgcagatttcttaatgttccaaatggaaaaaaatggtgcattacatgtgaccaatccaagcgatattcaaaaactagaaaaagtagctgtcactgataaaggtgtaacaaacaaaaaattagtcggaaaaagcgcttcagaaatcttaaaagaagcaggaatagcctgtgatttttcaccgcgtttaattattgtggaaacagaaaaaacgcatccttttgcaacagtagaattattaatgccaatcgttcctgttgttagagttcctaattttgaggaagcacttgaagttgctattgagttagaacaaggcttgcatcacacagcaacgatgcattcgcaaaatatttctagattaaataaagctgcacgagacatgcaaacatccatctttgtcaaaaatggtccttcatttgcgggattaggctttagaggagaaggtagcactactttcacaattgcaacgcctaccggagaaggaaccactacagcacgacattttgctagacgtcgccgttgtgttttaacagatggtttttcgattcgttaa

SEQ ID NO: 50 - protein, 469 aa, Listeria monocytogenes
MESLELEKLVKKVLLEKLAEQKGIPVKTMTKGAKSGVFDTVDEAVQAAVIAQNSYKEKSLEERRNVVKAIREALYPEIESIAARAVAETGMGNVADKILKNTLAIEKTPGVEDLYTEVATGDNGMTLYELSPYGVIGAVAPSTNPTETLICNTIGMLAAGNAVFYSPHPGAKNISLWLIEKLNTIVRESCGVDNLVVTVEKPSIQAAQEMMNHPKVPLLVITGGPGVVLQAMQSGKKVIGAGAGNPPSIVDETANIEKAAADIVDGASFDHNILCIAEKSVVAVDSIADFLMFQMEKNGALHVTNPSDIQKLEKVAVTDKGVTNKKLVGKSASEILKEAGIACDFSPRLIIVETEKTHPFATVELLMPIVPVVRVPNFEEALEVAIELEQGLHHTATMHSQNISRLNKAARDMQTSIFVKNGPSFAGLGFRGEGSTTFTIATPTGEGTTTARHFARRRRCVLTDGFSIR

SEQ ID NO: 51 - DNA, 1356 bp, Porphyromonas gingivalis
  CDS (1)..(1356)
atggaaatcaaagaaatggtgagccttgcgcgcaaggctcagaaggagtatcaagctacccataaccaagaagcagttgacaacatttgccgagctgcagcaaaagttatttatgaaaatgcagctattctggctcgcgaagcagtagacgaaaccggcatgggcgtttacgaacacaaagtggccaagaatcaaggcaaatccaaaggtgtttggtacaacctccacaataaaaaatcggttggtatcctcaatatagacgagcgtaccggtatgatcgagatcgcaaagcctatcggagttgtaggagccgtaacgccgacgaccaacccgatcgttactccgatgagcaatatcatctttgctcttaagacttgcaatgccatcattattgccccccaccccagatctaaaaaatgctctgcacacgcagttcgtctgatcaaagaagctatcgctccgttcaacgtaccggaaggtatggttcagatcatcgaagaacccagcatcgagaagacgcaggaactcatgggcgccgtagacgtagtagttgctacgggtggtatgggcatggtgaagtctgcatattcttcaggaaagccttctttcggtgttggagccggtaacgttcaggtgatcgtggatagcaacatcgatttcgaagctgctgcagaaaaaatcatcaccggtcgtgctttcgacaacggtatcatctgctcaggcgaacagagcatcatctacaacgaggctgacaaggaagcagttttcacagcattccgcaaccacggtgcatatttctgtgacgaagccgaaggagatcgtgctcgtgcagctatcttcgaaaatggagccatcgcgaaagatgtagtaggtcagagcgttgccttcattgccaagaaagcaaacatcaatatccccgagggtacccgtattctcgttgttgaagctcgcggcgtaggagcagaagacgttatctgtaaggaaaagatgtgtcccgtaatgtgcgccctcagctacaagcacttcgaagaaggtgtagaaatcgcacgtacgaacctcgccaacgaaggtaacggccacacttgtgctatccactccaacaatcaggcacacatcatcctcgcaggatcagagctgacggtatctcgtatcgtagtgaatgctccgagtgccactacagcaggcggtcacatccaaaacggccttgccgtaaccaatacgctcggatgcggatcatggggtaataactctatctccgagaacttcacttacaagcacctcctcaacatttcacgcatcgcaccgttgaattcaagcattcacatccccgatgacaaagaaatctgggaactctaa

SEQ ID NO: 52 - protein, 451 aa, Porphyromonas gingivalis
MEIKEMVSLARKAQKEYQATHNQEAVDNICRAAAKVIYENAAILAREAVDETGMGVYEHKVAKNQGKSKGVWYNLHNKKSVGILNIDERTGMIEIAKPIGVVGAVTPTTNPIVTPMSNIIFALKTCNAIIIAPHPRSKKCSAHAVRLIKEAIAPFNVPEGMVQIIEEPSIEKTQELMGAVDVVVATGGMGMVKSAYSSGKPSFGVGAGNVQVIVDSNIDFEAAAEKIITGRAFDNGIICSGEQSIIYNEADKEAVFTAFRNHGAYFCDEAEGDRARAAIFENGAIAKDVVGQSVAFIAKKANINIPEGTRILVVEARGVGAEDVICKEKMCPVMCALSYKHFEEGVEIARTNLANEGNGHTCAIHSNNQAHIILAGSELTVSRIVVNAPSATTAGGHIQNGLAVTNTLGCGSWGNNSISENFTYKHLLNISRIAPLNSSIHIPDDKEIWEL

SEQ ID NO: 53 - DNA, 1392 bp, Klebsiella oxytoca
  CDS (1)..(1392)
atgaatacttccgaactcgaaacccttattcgcaccattcttagcgaacagttaaccccggggcaaacgccggtccagccgcagggcaaagggatttttcagtccgtcagcgaggctatcgacgccgcgcaccaggcgttcttacgttatcagcagtgcccgctgaaaacccgcagcgccatcatcagcgcaatccgtcaggagctgacgccgcacctggccgcgctggcggaagagagcgccaatgaaacggggatgggcaacaaagaagataaactcttgaaaaacaaggccgcgctggacaacacgccgggcgtggaagatctcaccaccaccgcgctgaccggcgacggcggcatggtgctgtttgagtattcgccgtttggcgttatcgggtcggtcgcgccgagcaccaacccgacggaaaccatcatcaacaacagcatcagcatgctggctgcgggcaacagcgtctatttcagcccgcatccgggagcaaaaaaggtctctctgaagctgattagcatgattgaagagattgccttccgctgctgcggcatccgcaatctggtggtcaccgtggcggaaccgacgtttgaagccacccagcagatgatggcccatccgcgcattgcggtcctcgctatcaccggcggccccggcattgtggcgatgggcatgaagagcggtaaaaaagtgatcggcgccggcgcgggcaacccgccctgcatcgtcgatgaaacggcggatctggtcaaagcggcggaagatatcatcaacggcgcctcgttcgattacaacctgccctgcattgccgagaagagcctgattgtggtggagagcgtcgccgaacgtctggtgcagcaaatgcaaaccttcggcgcgctgctgctgagccctgccgacaccgacaaactccgcgccgcctgcctgccagagggccaggcgaataagaaactggtcggcaaaagcccatcggccatgctggaagcggccgggatcgccgttccggcgaaagcgccgcgtctgctgatagcgctggttaacgccgacgatccgtgggtcaccagcgagcagctgatgccgatgctgccggtggtgaaagtcagcgatttcgatagcgcgctggcgctggcgctgaaggttgaagaggggctgcatcataccgccattatgcactcgcaaaatgtctcgcgcctgaacctcgcggcccgcactctgcaaacctcgatattcgttaagaacggcccctcttatgccgggattggcgtcggcggcgaaggctttaccacctttactatcgccacgccgaccggtgaaggcacaacctcggcgcgcacctttgcccgctcccgtcgctgcgtgctgaccaacggcttttccattcgctaa

SEQ ID NO: 54 - protein, 463 aa, Klebsiella oxytoca
MNTSELETLIRTILSEQLTPGQTPVQPQGKGIFQSVSEAIDAAHQAFLRYQQCPLKTRSAIISAIRQELTPHLAALAEESANETGMGNKEDKLLKNKAALDNTPGVEDLTTTALTGDGGMVLFEYSPFGVIGSVAPSTNPTETIINNSISMLAAGNSVYFSPHPGAKKVSLKLISMIEEIAFRCCGIRNLVVTVAEPTFEATQQMMAHPRIAVLAITGGPGIVAMGMKSGKKVIGAGAGNPPCIVDETADLVKAAEDIINGASFDYNLPCIAEKSLIVVESVAERLVQQMQTFGALLLSPADTDKLRAACLPEGQANKKLVGKSPSAMLEAAGIAVPAKAPRLLIALVNADDPWVTSEQLMPMLPVVKVSDFDSALALALKVEEGLHHTAIMHSQNVSRLNLAARTLQTSIFVKNGPSYAGIGVGGEGFTTFTIATPTGEGTTSARTFARSRRCVLTNGFSIR

SEQ ID NO: 55 - DNA, 1590 bp, Rhodospirillum rubrum
  CDS (1)..(1590)
atgaatgacggacagatcgccgccgccgtggcaaaggttctggaagcctatggcgttcccgccgacccgtctgcggccgctcccgccccggccgcgccggtcgccccggccgcgccgacggccggaagcgtttccgagatgatcgcgcgcggcatcgccaaggcgtcgagcgacgatcagatcgcccagatcgtggccaaggtggttggcgactatagtgcccaggccgccaagccggccgtggtcccgggcgccgccgcgtcgaccgaggccggcgatggggttttcgacaccatggacgccgccgtcgacgccgccgtcctggcccagcagcagtatcttctgtgctcgatgaccgatcgccagcgtttcgtcgacggcatccgcgaggtgatcttgcaaaaagacaccctggagctgatctcgcggatggcggccgaagagaccggcatgggcaattacgagcacaagctgatcaagaaccgccttgccgccgaaaagacgccgggcaccgaggatctgaccaccgaggccttcagcggcgatgacggcctgaccctggtcgaatactcgccctttggcgccatcggcgccgtcgcgcccacgaccaatcccaccgaaaccatcatctgcaattccatcggcatgctggccgccggcaacagcgtgatcttcagcccccatccgcgggcgacgaaggtttccttgctgaccgtgaagctgatcaatcagaagctggcctgcctgggcgccccggccaatctggtcgtcaccgtcagcaagccctcggtcgagaacaccaacgccatgatggcgcatccgaagatccgcatgctggtcgccaccggcggtccggggatcgtcaaggcggtgatgtcgacgggcaagaaggccatcggcgccggcgcgggcaatccgccggtcgtcgtcgacgagaccgccgatatcgaaaaggccgccctcgatatcatcaacggctgtagcttcgataacaacctgccctgcatcgccgaaaaagagatcatcgccgtcgcccagatcgccgattacctgatcttttcgatgaagaagcagggcgcctatcagatcaccgaccccgccgtgctgcgcaagctccaggacctcgtcctgacggccaagggcggtccgcaaacctcctgcgtgggcaaaagcgccgtctggctgctcaacaagatcggcatcgaggttgatagcagcgtcaaggtcatcttgatggaggtgcccaaggagcatcccttcgtgcaggaagagctgatgatgccgatcctgccgctggtccgggtgagcgatgtcgacgaggccatcgccgtggccatcgaggtcgagcacggcaaccgccacaccgccatcatgcattcgaccaatgtgcgcaagctgaccaagatggccaagctgatccagacgacgatcttcgtgaagaacggcccgtcctacgccggtcttggcgtgggtggcgaaggctatacgaccttcaccatcgccggccccaccggcgagggtctgacctcggccaagtcgttcgcgcgcaaaagaaaatgcgtgatggtcgaagcgctcaacatccgctga

SEQ ID NO: 56 - protein, 529 aa, Rhodospirillum rubrum
MNDGQIAAAVAKVLEAYGVPADPSAAAPAPAAPVAPAAPTAGSVSEMIARGIAKASSDDQIAQIVAKVVGDYSAQAAKPAVVPGAAASTEAGDGVFDTMDAAVDAAVLAQQQYLLCSMTDRQRFVDGIREVILQKDTLELISRMAAEETGMGNYEHKLIKNRLAAEKTPGTEDLTTEAFSGDDGLTLVEYSPFGAIGAVAPTTNPTETIICNSIGMLAAGNSVIFSPHPRATKVSLLTVKLINQKLACLGAPANLVVTVSKPSVENTNAMMAHPKIRMLVATGGPGIVKAVMSTGKKAIGAGAGNPPVVVDETADIEKAALDIINGCSFDNNLPCIAEKEIIAVAQIADYLIFSMKKQGAYQITDPAVLRKLQDLVLTAKGGPQTSCVGKSAVWLLNKIGIEVDSSVKVILMEVPKEHPFVQEELMMPILPLVRVSDVDEAIAVAIEVEHGNRHTAIMHSTNVRKLTKMAKLIQTTIFVKNGPSYAGLGVGGEGYTTFTIAGPTGEGLTSAKSFARKRKCVMVEALNIR

SEQ ID NO: 57 - DNA, 1404 bp, Salmonella enterica
  CDS (1)..(1404)
atgaatcaacaggatattgaacaggtggtgaaagcggtactgctgaaaatgaaagacagcagccagccggccagtactgttcatgagatgggcgtttttgcctccctggatgacgcagtggcggcagcaaaacgggcccagcaggggctgaagagcgtggcgatgcgccagcttgccattcatgccattcgcgaagcgggcgaaaaacacgcccgagaattagcggaacttgccgtcagcgaaaccggcatgggacgcgttgacgataaatttgccaaaaacgtggcgcaggcgcgcggcacgccgggcgtcgagtgtttatctccgcaggtgctgaccggcgataacggcctgacgctgattgaaaatgcaccgtggggcgtggtggcctcggtcacgccgtccaccaatccggcggcgacggtgattaataacgccatcagcctgattgccgcaggcaacagcgtcgtgtttgccccgcatccggcggcgaaaaaggtctctcagcgggcaatcactctcctcaatcaggcggtggtcgccgccggtggtccggaaaatttgctggtcaccgtggcgaacccggatatcgaaaccgcccagcggttgttcaaatatcccggtattggcctgctggtggtgactggcggcgaacgggtggtggacgcggcgcgtaaacatactaataaacgcctgatcgccgccggtgccggcaatcccccggtggtggtggatgagaccgccgacctgccgcgcgctgcacaatccatcgtcaaaggcgcgtcgtttgataacaacatcatctgtgccgacgaaaaagtgctgattgtggtggatagcgtcgccgacgagctgatgcgtctgatggaaggtcagcacgcggtgaaactgaccgccgctcaggccgaacagctccagccggtgctgctgaagaatatcgatgaacgcggcaaaggcaccgtcagccgtgactgggtcggtcgtgacgcgggcaagatcgccgcagccatcggcctgaacgtcccggatcaaacgcgcctgctgtttgtggaaacgcccgccaaccatccgtttgccgttactgaaatgatgatgccggtactgccggtggtgcgggtcgccaacgtcgaagaagccatcgccctggcggttcagcttgaaggcggttgccaccatacggcggcgatgcactcgcgcaatatcgacaacatgaaccagatggcgaacgccatcgacaccagcattttcgtcaaaaacgggccgtgcattgccgggcttggattgggcggagaaggctggaccaccatgactatcactacgccaaccggcgaaggggtgaccagcgcgcgtacctttgtgcggctgcgtcgatgcgtgctggtggatgcgtttcgcattgtataa

SEQ ID NO: 58 - protein, 467 aa, Salmonella enterica
MNQQDIEQVVKAVLLKMKDSSQPASTVHEMGVFASLDDAVAAAKRAQQGLKSVAMRQLAIHAIREAGEKHARELAELAVSETGMGRVDDKFAKNVAQARGTPGVECLSPQVLTGDNGLTLIENAPWGVVASVTPSTNPAATVINNAISLIAAGNSVVFAPHPAAKKVSQRAITLLNQAVVAAGGPENLLVTVANPDIETAQRLFKYPGIGLLVVTGGERVVDAARKHTNKRLIAAGAGNPPVVVDETADLPRAAQSIVKGASFDNNIICADEKVLIVVDSVADELMRLMEGQHAVKLTAAQAEQLQPVLLKNIDERGKGTVSRDWVGRDAGKIAAAIGLNVPDQTRLLFVETPANHPFAVTEMMMPVLPVVRVANVEEAIALAVQLEGGCHHTAAMHSRNIDNMNQMANAIDTSIFVKNGPCIAGLGLGGEGWTTMTITTPTGEGVTSARTFVRLRRCVLVDAFRIV

SEQ ID NO: 59 - DNA, 1404 bp, Salmonella enterica
  CDS (1)..(1404)
atgaatcaacaggatattgaacaggtggtgaaagcggtgctgctgaaaatgaaagacagcagccagccggccagcactgttcatgagatgggcgtttttgcctccctggatgacgaagtggcggcagcaaaacgggcccagcaggggctgaagagcgtggcgatgcgccagcttgccattcatgccattcgcgaggcgggcgaaaaacacgcccgagaattagcggaacttgccgtcagcgaaaccggcatgggacgcgttgacgataaatttgccaaaaacgtggcgcaggcgcgcggcacgccgggcgtcgagtgtttatctccgcaggtgctgaccggcgataacggcctgacattgattgaaaatgcgccgtggggcgtggtggcctcggtcacgccgtccaccaatccggcggcgacggtgatcaataacgccatcagcctgattgccgcaggcaacagcgtcgtgtttgccccgcatccggcggcgaaaaaggtctctcagcgggcaatcaccctcctcaatcaggcggtggtcgccgccggcggcccggaaaatctgctggtcaccgtggcgaacccggatatcgaaaccgcccagcggctgttcaagtaccccggtattggcctgctggtggtgaccggcggcgaagcggtggtggacgcggcgcgcaaacatactaataaacgcctgatcgccgccggtgccggcaatcccccggtggtggtggatgagaccgccgacctgccgcgcgccgcacaatccatcgtcaaaggcgcgtcgtttgataacaacatcatctgtgccgacgaaaaagtgctgattgtggtggatagcgtcgccgacgagctgatgcgtctgatggaaggtcagcacgcggtgaaactgactgccgctcaggccgaacagctccagccggtgctgctgaagaatattgatgagcgcggcaaaggcaccgtcagccgtgactgggtcggtcgtgacgcgggcaaaatcgccgcagccattggcctgaacgtcccggatcaaacgcgcctgctatttgtggaaacgccagctaaccatccgtttgccgtcactgaaatgatgatgccggtactgccggtggtgcgggtcgccaacgtcgaagatgccatcgccctggcggtacaacttgaaggcggttgccaccatacggcggcgatgcactcgcgcaatatcgacaacatgaaccagatggcgaacgccatcgacaccagcattttcgtcaaaaacgggccgtgcattgccgggcttggattgggcggagaaggctggaccaccatgactatcactacgccaaccggcgaaggggtgaccagcgcgcgtacctttgtgcggctgcgtcgatgcgtgctggtggatgcgtttcgcattgtataa

SEQ ID NO: 60 - protein, 467 aa, Salmonella enterica
MNQQDIEQVVKAVLLKMKDSSQPASTVHEMGVFASLDDEVAAAKRAQQGLKSVAMRQLAIHAIREAGEKHARELAELAVSETGMGRVDDKFAKNVAQARGTPGVECLSPQVLTGDNGLTLIENAPWGVVASVTPSTNPAATVINNAISLIAAGNSVVFAPHPAAKKVSQRAITLLNQAVVAAGGPENLLVTVANPDIETAQRLFKYPGIGLLVVTGGEAVVDAARKHTNKRLIAAGAGNPPVVVDETADLPRAAQSIVKGASFDNNIICADEKVLIVVDSVADELMRLMEGQHAVKLTAAQAEQLQPVLLKNIDERGKGTVSRDWVGRDAGKIAAAIGLNVPDQTRLLFVETPANHPFAVTEMMMPVLPVVRVANVEDAIALAVQLEGGCHHTAAMHSRNIDNMNQMANAIDTSIFVKNGPCIAGLGLGGEGWTTMTITTPTGEGVTSARTFVRLRRCVLVDAFRIV

SEQ ID NO: 61 - DNA, 1404 bp, Klebsiella oxytoca
  CDS (1)..(1404)
atgaatcaacaggatattgaacaggtggtgaaagcggtactgctgaaaatgacggccagcagccagccggacgacgccgttcatgagatgggcgtctttgcctccctggatgacgccgtcgcggcggcaaaggtggcccagcaggggctgaaaagcgtggcgatgcgccagctggcgattcacgccatccgtgaggcgggcgaaaaacacgccagagaattagcggagcttgccgtcagcgaaaccggcatgggtcgcgttgaagataaatttgccaaaaacgtggcccaggcgcgcggcacgccgggcgtggagtgcctctctccgcaggtgctcaccggcgataacggcctgacgctgattgaaaacgcgccctggggcgtggtggcctcggtcaccccctccaccaacccggcggcgacggtaattaataacgccatcagcctgattgccgcgggcaatagcgtggtgttcgccccgcatccggcggcgaaaagggtttcccagcgggcgattatgctgctcaaccaggcggtgattgccgcgggcggtccggccaatctgctggtcaccgtggcgaacccggacatcgaaacggcccagcggctgtttaagtaccccggtattggcctgctggtggtcaccggcggtgaagcggtggtggagtccgcgcgcaagcataccaataaacgtctgattgccgccggcgccggtaacccgccggtagtggtcgacgaaacggccgacctggcgcgcgccgcgcggtccatcgtcaaaggcgcctcgtttgataacaacatcatctgcgccgacgaaaaggtgctgattgtggtcgatagcgtcgccgacgagctgatgcgcctgatggaaggccagcaggcggtgaagctcagcgcggcccaggccgagcagcttcagccggtgctgctgaagaatatcgatgagcgcggcaaaggtacggtcagccgtgactgggtgggacgtgacgcgagcaagattgccgccgccatcggcctgaacgtgccgccgcagacgcgcctgctgttcgtggaaaccccggccagccatccatttgccgtgacggaactgatgatgccggtgctccctgtcgtacgggtggccagcgtcgatgacgctatcgcgctggcggtgcagctggagggcggctgccaccatacggcggcgatgcactcgcgcaatatcgacaacatgaatcagatggcgaacgccattgataccagcattttcgtcaaaaacgggccgtgcatcgccgggctcgggctgggcggagagggctggaccaccatgaccatcaccacgccaaccggggagggggtgaccagtgcgcgcaccttcgttcgcctgcggcgctgcgtgctggtggatgccttccgaattgtataa

SEQ ID NO: 62 - protein, 467 aa, Klebsiella oxytoca
MNQQDIEQVVKAVLLKMTASSQPDDAVHEMGVFASLDDAVAAAKVAQQGLKSVAMRQLAIHAIREAGEKHARELAELAVSETGMGRVEDKFAKNVAQARGTPGVECLSPQVLTGDNGLTLIENAPWGVVASVTPSTNPAATVINNAISLIAAGNSVVFAPHPAAKRVSQRAIMLLNQAVIAAGGPANLLVTVANPDIETAQRLFKYPGIGLLVVTGGEAVVESARKHTNKRLIAAGAGNPPVVVDETADLARAARSIVKGASFDNNIICADEKVLIVVDSVADELMRLMEGQQAVKLSAAQAEQLQPVLLKNIDERGKGTVSRDWVGRDASKIAAAIGLNVPPQTRLLFVETPASHPFAVTELMMPVLPVVRVASVDDAIALAVQLEGGCHHTAAMHSRNIDNMNQMANAIDTSIFVKNGPCIAGLGLGGEGWTTMTITTPTGEGVTSARTFVRLRRCVLVDAFRIV

SEQ ID NO: 63 - DNA, 1392 bp, Klebsiella oxytoca
  CDS (1)..(1392)
atgaatacttctgaactcgaaacgcttattcgcaccattcttagcgaacagttaaccccagcccaaacgccaaacccggcgcagggcaaagggatttttcagtccgtgagcgaggccattgatgccgcgcatcaggcgttcttacgttatcagcagtgcccgctaaaaacccgcagcgccatcattagcgcaatgcgtcaggagctgacgccgcatctggccgccctggcggaagagagcgccaatgaaacggggatgggcaacaaagaagataaatttttgaaaaacaaagctgcgctggacaacacgccgggcgtggaagatctcaccaccacggcactgaccggcgacggcggcatggtgctgtttgagtattcgccatttggcgttatcgggtcggtcgcaccgagcaccaacccgacggaaaccatcattaataacagcatcagcatgctggcggcgggcaatagcgtctacttcagcccgcatccgggagcaaaaaaggtctcactgaagctgattagcatgattgaagagattgttttccgctgctgcggcatccgcaatctggtggttaccgtagcagaacccactttcgaagccacccagcagatgatggcccatccgcgcattgcggttctggccattaccggcggcccgggtattgtggcgatgggcatgaagagcggtaagaaagtcattggcgctggcgcgggtaacccgccctgcatcgttgatgaaacggcggatctggttaaagcgtcggaagatatcatcaacggcgcctcgttcgattacaacctgccctgcattgccgagaagagcctgattgtggtggagagcgtcgccgaacgtctggtgcagcaaatgcaaaccttcggcgcgctgctgctgagccctgccgacactgacaaactccgggccgcctgcctgcctgaaggccaggcgaataaaaaactggtcggcaaaagcccatcagccatgctggaagcggccgggatcgccgttccgtcgaaagcgccgcgtctactgatagcgctggttaacgccgacgatccgtgggttaccagcgagcagctgatgccgatgctgccgatagtgaaagttagcgatttcgatagcgcgctggccctggcgctgaaggttgaagagggactgcatcataccgccattatgcactcgcagaacgtgtcgcgcctgaacctcgcggcccgcaccctgcaaacctcgatattcgtcaagaacggcccctcttatgccgggatcggcgtcggcggcgaaggctttaccacctttactatcgccacgccgaccggtgaagggacaacctcggcgcgcacgtttgcccgctcccgtcgctgcgtgctgaccaacggcttttccattcgctaa

SEQ ID NO: 64 - protein, 463 aa, Klebsiella oxytoca
MNTSELETLIRTILSEQLTPAQTPNPAQGKGIFQSVSEAIDAAHQAFLRYQQCPLKTRSAIISAMRQELTPHLAALAEESANETGMGNKEDKFLKNKAALDNTPGVEDLTTTALTGDGGMVLFEYSPFGVIGSVAPSTNPTETIINNSISMLAAGNSVYFSPHPGAKKVSLKLISMIEEIVFRCCGIRNLVVTVAEPTFEATQQMMAHPRIAVLAITGGPGIVAMGMKSGKKVIGAGAGNPPCIVDETADLVKASEDIINGASFDYNLPCIAEKSLIVVESVAERLVQQMQTFGALLLSPADTDKLRAACLPEGQANKKLVGKSPSAMLEAAGIAVPSKAPRLLIALVNADDPWVTSEQLMPMLPIVKVSDFDSALALALKVEEGLHHTAIMHSQNVSRLNLAARTLQTSIFVKNGPSYAGIGVGGEGFTTFTIATPTGEGTTSARTFARSRRCVLTNGFSIR

SEQ ID NO: 65 - DNA, 1392 bp, Klebsiella oxytoca
  CDS (1)..(1392)
atgaatacttctgaactcgaaacgcttattcgcaccattcttagcgaacagttgaccccagctcaaacgccaaacccggcgcagggcaaagggatttttcagtccgtgagcgaggccattgatgccgcgcatcaggcgttcttacgttatcagcagtgcccgctaaaaacccgcagcgccatcattagcgcaatgcgtcaggagctgacgccgcatctggccgccctggcggaagagagtgccaatgaaacggggatgggcaacaaagaagataaatttttgaaaaacaaagctgcgctggacaacacgccgggcgtggaagatctcaccaccgcggcactgagcggcgacggcggcatggtgctgtttgaatattcgccatttggcgttatcgggtcggtcgcaccgagcaccaacccgacggaaaccatcattaataacagcatcagcatgctggcggcgggcaatagcgtctacttcagcccgcatccgggagcaaaaaaggtctcactgaagctgattagcatgattgaagagattgttttccgctgctgcggcatccgcaatctggtggttaccgtagcagaacccactttcgaagccacccagcagatgatggcccatccgcgcattgcggttctggccattaccggcggcccgggtattgtggcgatgggcatgaagagcggtaagaaagtcattggcgctggcgcgggtaacccgccctgcatcgttgatgaaacggcggatctggttaaagcggcggaagatatcatcaacggcgcctcgttcgattacaacctgccctgcattgccgagaagagcctgattgtggtggagagcgtcgccgaacgtctggtgcagcaaatgcaaaccttcggcgcgctgctgctgagccctgccgacaccgacaaactccgcgccgcctgcctgcctgaaggccaggcgaataaaaaactggtcggcaaaagcccatcggccatgctggaagcggccggcatcgccgttccggcgaaagcgccgcgtctgctgatagcgctggttaacgccgacgatccgtgggtcaccagcgagcagctgatgccgatgctgccgatagtgaaagttagcgatttcgatagcgcgctggccctggcgctgaaggttgaagaaggactgcatcataccgccattatgcactcgcagaacgtgtcgcgcctgaacctcgcggcccgcaccctgcaaacctcgatattcgtcaagaacggcccctcttatgccgggatcggcgtcggcggcgaaggctttaccacctttactatcgccacgccgaccggtgaagggacaacctcggcgcgcacgtttgcccgctcccgtcgctgcgtgctgaccaacggcttttccattcgctaa

SEQ ID NO: 66 - protein, 463 aa, Klebsiella oxytoca
MNTSELETLIRTILSEQLTPAQTPNPAQGKGIFQSVSEAIDAAHQAFLRYQQCPLKTRSAIISAMRQELTPHLAALAEESANETGMGNKEDKFLKNKAALDNTPGVEDLTTAALSGDGGMVLFEYSPFGVIGSVAPSTNPTETIINNSISMLAAGNSVYFSPHPGAKKVSLKLISMIEEIVFRCCGIRNLVVTVAEPTFEATQQMMAHPRIAVLAITGGPGIVAMGMKSGKKVIGAGAGNPPCIVDETADLVKAAEDIINGASFDYNLPCIAEKSLIVVESVAERLVQQMQTFGALLLSPADTDKLRAACLPEGQANKKLVGKSPSAMLEAAGIAVPAKAPRLLIALVNADDPWVTSEQLMPMLPIVKVSDFDSALALALKVEEGLHHTAIMHSQNVSRLNLAARTLQTSIFVKNGPSYAGIGVGGEGFTTFTIATPTGEGTTSARTFARSRRCVLTNGFSIR

SEQ ID NO: 67 - DNA, 1410 bp, Listeria innocua
  CDS (1)..(1410)
atggaatcattagaactcgaacaactggtaaaaaaagttctcttagaaaaattagcagaacaaaaagaagtaccaacaaaaacaactacacaaggcgcgaaaagtggcgtttttgatacagttgacgaggctgttcaagcagcagttatagcgcagaattgctataaagaaaaatcacttgaagaacgccgcaatgttgtaaaagcaattcgtgaagcactttatccagaaattgaaacaattgcgacaagagcagttgcagagactggtatgggaaatgtgacagataaaattttgaaaaacacgttagcaatcgaaaaaacgccaggggtagaagatttatatacagaagtagctacaggtgataacggtatgacactatatgaactctctccgtatggcgtaattggtgcagtagcgccgagcacaaacccaacggaaacattgatttgtaattcaatcggtatgctcgcagctggaaatgccgttttttatagccctcatccaggggcaaaaaacatttcactgtggttgattgaaaaactaaacacaattgttcgcgatagttgtggtatagataatctaattgtcaccgtggctaaaccatccatccaagcagctcaagaaatgatgaaccatccaaaagtaccgctacttgttattacaggtggtccgggcgttgttctccaagcgatgcaatcaggtaaaaaagtgattggagcaggagcagggaacccgccttctattgttgacgaaacagctaatatcgaaaaagcggctgctgacatcgtagacggagcatcttttgaccataatattttatgtattgctgaaaaaagtgtggtagctgttgatagcattgctgatttcttgttattccaaatggaaaaaaatggtgcccttcatgttactaatccaagtgatattcaaaaattagaaaaagtagccgttaccgataaaggtgtaactaataaaaaattagtcggaaaaagtgcaactgaaatcttaaaagaagcaggaatagcttgtgattttacaccacgtttaatcattgtggaaacggagaaatctcatccatttgcaacagtagagctattaatgccaatcgttccagttgtaagggtgcctgattttgacgaagcccttgaagtggctattgaactcgaacaaggcttacatcatacagcaacaatgcattcacaaaatatctcgagattaaacaaagctgcaagagatatgcaaacttccatctttgtcaaaaatggtccgtcctttgcgggattaggctttagaggagaaggtagtactactttcactattgcaacgcctactggagaaggaacaactacagcacgtcattttgctagacgccgccgctgtgttttaacagatggtttttcgattcgttaa

SEQ ID NO: 68 - protein, 469 aa, Listeria innocua
MESLELEQLVKKVLLEKLAEQKEVPTKTTTQGAKSGVFDTVDEAVQAAVIAQNCYKEKSLEERRNVVKAIREALYPEIETIATRAVAETGMGNVTDKILKNTLAIEKTPGVEDLYTEVATGDNGMTLYELSPYGVIGAVAPSTNPTETLICNSIGMLAAGNAVFYSPHPGAKNISLWLIEKLNTIVRDSCGIDNLIVTVAKPSIQAAQEMMNHPKVPLLVITGGPGVVLQAMQSGKKVIGAGAGNPPSIVDETANIEKAAADIVDGASFDHNILCIAEKSVVAVDSIADFLLFQMEKNGALHVTNPSDIQKLEKVAVTDKGVTNKKLVGKSATEILKEAGIACDFTPRLIIVETEKSHPFATVELLMPIVPVVRVPDFDEALEVAIELEQGLHHTATMHSQNISRLNKAARDMQTSIFVKNGPSFAGLGFRGEGSTTFTIATPTGEGTTTARHFARRRRCVLTDGFSIR

SEQ ID NO: 69 - DNA, 1395 bp, Listeria innocua
  CDS (1)..(1395)
atgaatacttctgaactcgaaacccttattcgcaccattcttagcgagcaattaaccacgccagcgcaaacgccgatccagccgcagggcaaagggattttccagtccgtcagcgaggccatcgacgccgcgcaccaggcgttcttacgttatcagcagtgcccgttgaaaacccgcagcgccatcatcagcgcgatacgtcaggagctgacgccgctgctgaccgcgctggcggaagagagcgccaatgaaacgggaatgggcaacaaagaagataaatacctgaaaaacaaggctgcgctggacaacacccccggcgtggaagatctcaccaccaccgcgctgaccggcgacggcggtatggtgctgtttgagtattcgccgtttggcgtaatcgggtcggtcgcaccgagcaccaacccaacggagaccatcatcaacaacagcatcagcatgctggcggcgggcaacagcgtctacttcagcccgcatccgggagcgaaaaaggtctcactcaagctcatcagcctgattgaagagattgccttccgctgctgcggcatccgcaacctggtggtcaccgtggcggaaccgacgtttgaagcgacccaacagatgatggcccatccgcgcattgcggtcctcgctattaccggcggccccggcattgtggcgatgggcatgaagagcggtaaaaaagtgattggtgctggcgcgggcaaccccccctgcatcgttgatgaaaccgctgacctggtcaaagcggcggaggatatcatcaacggcgcctcgttcgattacaacctgccctgcattgccgagaaaagcctgattgtggtggagagcgtcgctgaacgtctggtacagcaaatgcaaaccttcggcgcgctgctgctaagcccggccgataccgacaagctccgcgccgcctgcctgccggaaggcctggccaataagaagctggtcggaaaaagcccatcggcgctgctggaagccgccgggatcgccgttccggcgaaagcgccgcgcttgctgatcgctatcgtcaacgccgacgacccgtgggtcaccagcgaacagctgatgccgatgctgccgatcgtgaaagtcagcgatttcgatagcgcgctggccctggcgctgaaggttgaagagggactgcatcataccgccattatgcactcgcaaaatgtgtcgcgcctgaacctcgcagcccgcaccctgcaaacctcgattttcgtcaagaacggcccctcttatgccgggatcggcgtcggcggtgaaggctttaccacctttaccattgccacgccgaccggcgaagggacgacctcggcgcgcacctttgcccgctcccgtcgctgcgtgctgaccaacggcttttccattcgctaa

SEQ ID NO: 70 - protein, 464 aa, Listeria innocua
MNTSELETLIRTILSEQLTTPAQTPIQPQGKGIFQSVSEAIDAAHQAFLRYQQCPLKTRSAIISAIRQELTPLLTALAEESANETGMGNKEDKYLKNKAALDNTPGVEDLTTTALTGDGGMVLFEYSPFGVIGSVAPSTNPTETIINNSISMLAAGNSVYFSPHPGAKKVSLKLISLIEEIAFRCCGIRNLVVTVAEPTFEATQQMMAHPRIAVLAITGGPGIVAMGMKSGKKVIGAGAGNPPCIVDETADLVKAAEDIINGASFDYNLPCIAEKSLIVVESVAERLVQQMQTFGALLLSPADTDKLRAACLPEGLANKKLVGKSPSALLEAAGIAVPAKAPRLLIAIVNADDPWVTSEQLMPMLPIVKVSDFDSALALALKVEEGLHHTAIMHSQNVSRLNLAARTLQTSIFVKNGPSYAGIGVGGEGFTTFTIATPTGEGTTSARTFARSRRCVLTNGFSIR

SEQ ID NO: 71 - DNA, 1410 bp, Listeria innocua
  CDS (1)..(1410)
atggaatcattagaactcgaacaactggtaaaaaaagttctcttagaaaaattagcagaacaaaaagaagtaccaacaaaaacaactacacaaggcgcgaaaagtggcgtttttgatacagttgacgaggctgttcaagcagcagttatagcgcagaattgctataaagaaaaatcacttgaagaacgccgcaatgttgtaaaagcaattcgtgaagcactttatccagaaattgaaacaattgcgacaagagcagttgcagagactggtatgggaaatgtgacagataaaattttgaaaaacacgttagcaatcgaaaaaacgccaggggtagaagatttatatacagaagtagctacaggtgataacggtatgacactatatgaactctctccatatggcgtaattggtgcagtagcgccgagcacgaacccaacggaaacattaatttgtaattcaatcggtatgctcgcagctggaaatgccgttttttatagccctcatccaggggcaaaaaacatttcactgtggttgattgaaaaactaaacacaattgttcgcgatagttgtggtatagataatctaattgtcaccgtggctaaaccatccatccaagcagctcaagaaatgatgaaccatccaaaagtaccgctacttgttattacaggtggtccgggtgttgttcttcaagcgatgcaatcaggtaaaaaagtgattggagcaggagcaggaaacccgccttctattgttgacgaaacagctaacatcgaaaaagcggctgctgatatcgtagacggtgcctcttttgatcataatattttatgtattgctgaaaaaagtgtggtagctgttgatagcattgctgatttcttgttattccaaatggaaaaaaatggtgctcttcatgttactaatccaagtgatattcaaaaattagaaaaagtagccgttaccgataaaggtgtaactaataaaaaattagtcggaaaaagtgcaactgaaatcttaaaagaagcaggaatagcttgtgatttcacaccacgtttaatcattgtggaaacggagaaatctcatccatttgcaacagtagagctattaatgccaatcgttccagttgtaagagtgcctgattttgacaaagcccttgaagtggctattgaactcgaacaaggtttacatcatacagcgacaatgcattcacaaaatatctcgagattaaacaaagctgcaagagatatgcaaacttccatctttgtcaaaaatggtccttcatttgcgggattaggctttagaggagaaggtagcactactttcactattgcaacgcctaccggagaaggaacaactacagcacgtcattttgctagacgtcgccgttgtgttttaacagatggtttttcgattcgttaa

SEQ ID NO: 72 - protein, 469 aa, Listeria innocua
MESLELEQLVKKVLLEKLAEQKEVPTKTTTQGAKSGVFDTVDEAVQAAVIAQNCYKEKSLEERRNVVKAIREALYPEIETIATRAVAETGMGNVTDKILKNTLAIEKTPGVEDLYTEVATGDNGMTLYELSPYGVIGAVAPSTNPTETLICNSIGMLAAGNAVFYSPHPGAKNISLWLIEKLNTIVRDSCGIDNLIVTVAKPSIQAAQEMMNHPKVPLLVITGGPGVVLQAMQSGKKVIGAGAGNPPSIVDETANIEKAAADIVDGASFDHNILCIAEKSVVAVDSIADFLLFQMEKNGALHVTNPSDIQKLEKVAVTDKGVTNKKLVGKSATEILKEAGIACDFTPRLIIVETEKSHPFATVELLMPIVPVVRVPDFDKALEVAIELEQGLHHTATMHSQNISRLNKAARDMQTSIFVKNGPSFAGLGFRGEGSTTFTIATPTGEGTTTARHFARRRRCVLTDGFSIR

SEQ ID NO: 73 - DNA, 1410 bp, Listeria innocua
  CDS (1)..(1410)
atggaatcattagaactcgaacaactggtaaaaaaagttctcttagaaaaattagcagaacaaaaagaagtaccaacaaaaacaactacacaaggcgcgaaaagtggcgtttttgatacagttgacgaggctgttcaagcagcagttatagcgcagaattgctataaagaaaaatcacttgaagaacgccgcaatgttgtaaaagcaattcgtgaagcactttatccagaaattgaaacaattgcgacaagagcagttgcagagactggtatgggaaatgtgacagataaaattttgaaaaacacattagcaattgaaaaaacaccaggggtagaagatttatatacagaagtagctactggtgataacggtatgacactgtatgaactctctccatatggcgtaattggtgcagtagcgccgagcacaaacccaacggaaacattgatttgtaattcaatcggtatgctcgcagctggaaatgccgttttttatagtcctcatccaggggcaaaaaacatttcactgtggttgattgaaaagctaaacacaattgttcgcgaaagttgtggtattgataaccttattgtaacggtggctaaaccatccattcaagcagctcaagaaatgatgaatcatccaaaagtaccgctacttgttattacaggtggtccgggcgttgttctccaagcgatgcaatcaggtaaaaaagtgattggagcaggagcagggaacccgccttctattgttgacgaaacagctaatatcgaaaaagcggctgctgacatcgtagacggtgcctcttttgatcataatattttatgtattgctgaaaaaagtgtggtagctgttgatagcattgctgatttcttgttattccaaatggaaaaaaatggtgctcttcatgttactaatccaagtgatattcaaaaattagaaaaagtagccgttaccgataaaggtgtaactaataaaaaattagtcggaaaaagtgcaactgaaatcttaaaagaagcaggaatagcttgtgattttacaccacgtttaatcattgtggaaacggagaaatctcatccatttgcaacagtagagctattaatgccaatcgttccagttgtaagagtgcctgattttgacgaagcccttgaagtggctattgaactcgaacaaggcttacatcatacagcaacaatgcattcacaaaatatctcgagattaaacaaagctgcaagagatatgcaaacttccatctttgtcaaaaatggtccttcctttgcgggattaggctttagaggagaaggtagtactactttcactattgcaacgcctaccggagaaggaacaactacagcacgtcattttgctagacgccgccgttgtgttttaacagatggtttttcgattcgttaa

SEQ ID NO: 74 - protein, 469 aa, Listeria innocua
MESLELEQLVKKVLLEKLAEQKEVPTKTTTQGAKSGVFDTVDEAVQAAVIAQNCYKEKSLEERRNVVKAIREALYPEIETIATRAVAETGMGNVTDKILKNTLAIEKTPGVEDLYTEVATGDNGMTLYELSPYGVIGAVAPSTNPTETLICNSIGMLAAGNAVFYSPHPGAKNISLWLIEKLNTIVRESCGIDNLIVTVAKPSIQAAQEMMNHPKVPLLVITGGPGVVLQAMQSGKKVIGAGAGNPPSIVDETANIEKAAADIVDGASFDHNILCIAEKSVVAVDSIADFLLFQMEKNGALHVTNPSDIQKLEKVAVTDKGVTNKKLVGKSATEILKEAGIACDFTPRLIIVETEKSHPFATVELLMPIVPVVRVPDFDEALEVAIELEQGLHHTATMHSQNISRLNKAARDMQTSIFVKNGPSFAGLGFRGEGSTTFTIATPTGEGTTTARHFARRRRCVLTDGFSIR

SEQ ID NO: 75 - DNA, 1410 bp, Listeria innocua
  CDS (1)..(1410)
atggaatcattagaactcgaaaaactggtgaaaaaagttctcttagaaaaattagcagaacaaaaagaagtaccaacaaaaacaactacacaaggcgtgaaaagtggcgtttttgatacagttgacgaggctgttcaagcagcagttatagcgcagaattgctataaagaaaaatcacttgaagaacgccgcaatgttgtaaaagcaattcgtgaagcactttatccagaaattgaaacaattgcgacaagagcagttgcggaaaccggaatgggtaatgtggcagataaaattttgaaaaacacgttagcaatcgaaaaaacgcccggggtagaagatttatatacagaagtagctactggtgataacggtatgacactgtatgaactctctccatatggcgtaattggtgcagtagcgccgagcacgaatccaacggaaacattaatttgtaattcaatcggaatgctcgcagctggaaatgccgttttttatagtcctcatccaggggcaaaaaacatttcactgtggttgattgaaaagctaaacacaattgttcgcgaaagttgtggtattgataatctaattgtaacggtggctaatccatccattcaagcagctcaagaaatgatgaatcatccaaaagtaccgctacttgttattacaggtggtccgggagttgttcttcaagcgatgcaatcaggtaaaaaagtgattggagcaggagcaggaaacccaccttctattgttgacgaaacagctaacatcgaaaaagcggctgctgatatcgtagacggtgcatcttttgatcataatattttatgtatcgctgaaaaaagtgtggtagccgttgatagcattgctgatttcttgttattccaaatggaaaaaaatggtgctcttcatgttactaatccaagtgatattcaaaaattagaaaaagtagccgttaccgataaaggtgtaactaataaaaaattagtcggaaaaagtgcaactgaaatcttaaaagaagcaggaatagcttgtgatttcacaccgcgtttaatcatagtggaaacggagaaatctcatccatttgcaacagtagagctattaatgccaattgttccagttgtaagagtgcctgattttgacgaagcccttgaagtggctattgaactcgaacaaggcttacatcatacagcgactatgcattcacaaaatatctcgagattaaacaaagctgcaagagacatgcaaacttccatctttgtaaaaaatggtccttcttttgcgggattaggctttagaggagaaggtagtactactttcactattgcaacgcctaccggagaaggaacaactacagcacgtcattttgctagacgccgccgttgtgttttaacagatggtttttcgattcgttaa

SEQ ID NO: 76 - protein, 469 aa, Listeria innocua
MESLELEKLVKKVLLEKLAEQKEVPTKTTTQGVKSGVFDTVDEAVQAAVIAQNCYKEKSLEERRNVVKAIREALYPEIETIATRAVAETGMGNVADKILKNTLAIEKTPGVEDLYTEVATGDNGMTLYELSPYGVIGAVAPSTNPTETLICNSIGMLAAGNAVFYSPHPGAKNISLWLIEKLNTIVRESCGIDNLIVTVANPSIQAAQEMMNHPKVPLLVITGGPGVVLQAMQSGKKVIGAGAGNPPSIVDETANIEKAAADIVDGASFDHNILCIAEKSVVAVDSIADFLLFQMEKNGALHVTNPSDIQKLEKVAVTDKGVTNKKLVGKSATEILKEAGIACDFTPRLIIVETEKSHPFATVELLMPIVPVVRVPDFDEALEVAIELEQGLHHTATMHSQNISRLNKAARDMQTSIFVKNGPSFAGLGFRGEGSTTFTIATPTGEGTTTARHFARRRRCVLTDGFSIR

SEQ ID NO: 77 - DNA, 1410 bp, Listeria innocua
  CDS (1)..(1410)
atggaatcattagaactcgaacaactggtgaaaaaagttctcttagaaaaattagcaggacaaaacgaagaaacaccaaaaaaaccaagccaaggtgccaaaagtggcattttcgatacagtggatgaggcagttcaagcagcagtaattgcgcaaaactgctacaaagaaaagtcactagaagaccgcagaaatgtagtaaaagcaattcgcgaagcactttatccggaaatcgaaaatattgcgacacgtgcggttgctgaaacaggtatgggtaacgtagccgataaaattttgaaaaatacgttagcaattgaaaaaacaccaggagtagaagatctctatacagaagtagctacaggcgataatggtatgacgctttatgaactttctccttatggtgttattggtgctgttgctccaagtacgaatccaacagaaacattaatttgcaacacaattggaatgcttgcagctggaaatgcagttttttatagcccacatccaggtgcaaaaaatatttcgctttggttgattgaaaaactaaatacgattgttcgcgaaagctgcgggattgataacctagtcgttacagttgaaaaaccatctattcaagcagcgcaagaaatgatgaatcatccaaaagtaccgttactagttatcactggcggtcctggtgttgttcttcaagcgatgcaatctggtaagaaagtaatcggagctggcgcgggaaatccaccttctatcgtagacgaaacagcgaatatcgaaaaagcagctgctgatatcgttgcgggtgcatcttttgatcataatattttatgtatcgcagaaaaaagtgtagtagcagtggatagcatcactgattttctattattccagatggaaaaaaatggtgccttacatgttacgaatccaagcgatattcgcaaactggaaaaagtggcagttacagaaaaaggcgttacaaacaagaagttagttggtaaaagcgcttcggaaattttaaaagaagcagggatagcatgtgattttacccctcgattaattattgttgaaacagataaatcccatccatttgcaacggtagaacttttaatgccgattgttccagtagtacgagttgctgattttgatcaagcacttgaagtagcacttgagttagaacaaggcttacatcacacggcaacaatgcattcacaaaatatctctagactgaacaaagcagcacgagatatgcaaacatccattttcgtgaaaaatggaccatcgtttgctggacttggctttggaggagaaggtagtgcaactttcactatcgctaccccaacaggtgaaggaactactactgcgcgacactttgctagacgccgtcgttgtgttttaacagatggtttttcgattcgttaa

SEQ ID NO: 78 - protein, 469 aa, Listeria innocua
MESLELEQLVKKVLLEKLAGQNEETPKKPSQGAKSGIFDTVDEAVQAAVIAQNCYKEKSLEDRRNVVKAIREALYPEIENIATRAVAETGMGNVADKILKNTLAIEKTPGVEDLYTEVATGDNGMTLYELSPYGVIGAVAPSTNPTETLICNTIGMLAAGNAVFYSPHPGAKNISLWLIEKLNTIVRESCGIDNLVVTVEKPSIQAAQEMMNHPKVPLLVITGGPGVVLQAMQSGKKVIGAGAGNPPSIVDETANIEKAAADIVAGASFDHNILCIAEKSVVAVDSITDFLLFQMEKNGALHVTNPSDIRKLEKVAVTEKGVTNKKLVGKSASEILKEAGIACDFTPRLIIVETDKSHPFATVELLMPIVPVVRVADFDQALEVALELEQGLHHTATMHSQNISRLNKAARDMQTSIFVKNGPSFAGLGFGGEGSATFTIATPTGEGTTTARHFARRRRCVLTDGFSIR

SEQ ID NO: 79 - DNA, 1410 bp, Listeria innocua
  CDS (1)..(1410)
atggaatcattagaactcgaacaactggtgaaaaaagttctcttagaaaaattagcaggacaaaacgaagaaacaccaaaaaaaccaagccaaggtgccaaaagtggcattttcgatacagtggatgaggcagttcaagcagcagtaattgcgcaaaactgctacaaagagaagtcactagaagaccgcagaaatgttgtaaaagcaattcgtgaagcactttatccggaaatcaaaaatattgcgacacgtgcggttgctgaaacaggtatgggtaacgtagccgataaaattttgaaaaatacgttagcaattgaaaaaacaccaggagtagaagatctctatacagaagtagctacaggcgataatggtatgacgctttatgaactttctccttatggtgttattggtgctgttgctccaagtacgaatccaacagaaacattaatttgcaacacaattggaatgcttgcagctggaaatgcagttttttatagcccgcatccaggtgcaaaaaatatttcgctttggttgattgaaaaactaaatacgattgttcgcgaaagctgcgggattgataacctagtcgttacagttgaaaaaccatctattcaagcagcgcaagaaatgatgaatcatccaaaagtaccgttactagttatcactggcggtcctggtgttgttcttcaagcgatgcaatctggtaagaaagtaatcggagcaggtgcgggaaatccaccttctatcgtagacgaaacagcgaatatcgaaaaagcagctgctgatatcgttgcgggtgcatcttttgatcataatattttatgtatcgcagaaaaaagcgtagtagcagtggatagcatcactgattttctcttattccaaatggaaaaaaatggtgcgttgcatgttacgaatccaagcgatattcgcaaactggaaaaagtggcagttaccgaaaaaggcgttaccaataagaagttagttggtaaaagcgcttcggaaattttaaaagaagcagggatagcatgtgattttacccctcgattaattattgttgaaacagatagatcccatccatttgcaacggtagaacttttaatgccgattgttccagtggtacgagttgctgattttgatcaagcacttgaagtagcacttgagttagaacaaggcttacatcacacggcaacaatgcattcacaaaatatctctagactgaacaaagcagcacgagatatgcaaacatccattttcgtgaaaaatggaccatcgtttgctggacttggctttggaggagaaggtagtgcaactttcactatcgctaccccaacaggtgaaggaactactactgcgcgacactttgctagacgccgtcgttgtgttttaacagatggtttttcgattcgttaa

SEQ ID NO: 80 - protein, 469 aa, Listeria innocua
MESLELEQLVKKVLLEKLAGQNEETPKKPSQGAKSGIFDTVDEAVQAAVIAQNCYKEKSLEDRRNVVKAIREALYPEIKNIATRAVAETGMGNVADKILKNTLAIEKTPGVEDLYTEVATGDNGMTLYELSPYGVIGAVAPSTNPTETLICNTIGMLAAGNAVFYSPHPGAKNISLWLIEKLNTIVRESCGIDNLVVTVEKPSIQAAQEMMNHPKVPLLVITGGPGVVLQAMQSGKKVIGAGAGNPPSIVDETANIEKAAADIVAGASFDHNILCIAEKSVVAVDSITDFLLFQMEKNGALHVTNPSDIRKLEKVAVTEKGVTNKKLVGKSASEILKEAGIACDFTPRLIIVETDRSHPFATVELLMPIVPVVRVADFDQALEVALELEQGLHHTATMHSQNISRLNKAARDMQTSIFVKNGPSFAGLGFGGEGSATFTIATPTGEGTTTARHFARRRRCVLTDGFSIR